SEQUENCE LISTING (19 sequences)

SEQ ID NO: 1 - DNA, 9 bp, artificial sequence
  synthetic oligonucleotide
cacattcca

SEQ ID NO: 2 - DNA, 267 bp, artificial sequence
  synthetic oligonucleotide
cacattccacacattccactgcaagcttgagacacattccacacattccactgcaagcttggccagtgccaagttgagacacattccacacattccactgcaagcttgagacacattccacacattccactgcaagcttctagagatctgcaggtcgaggtcgacggtatcgataagcttgggggtgggcgccggggggaccttaaagcctctgccccccaaggagcccttcccagacagccgccggcacccaccgctccgtgggac

SEQ ID NO: 3 - DNA, 32 bp, artificial sequence
  synthetic oligonucleotide
ggtggtggatcctgtacgggccagatatacgc

SEQ ID NO: 4 - DNA, 32 bp, artificial sequence
  synthetic oligonucleotide
ggtggtggatccgccagcttgggtctccctat

SEQ ID NO: 5 - DNA, 33 bp, artificial sequence
  synthetic oligonucleotide
ggtggtatcgatgccaccatggtcacaggaggc

SEQ ID NO: 6 - DNA, 38 bp, artificial sequence
  synthetic oligonucleotide
ggtggtatcgatagctagctcaggtttagacacagtag

SEQ ID NO: 7 - DNA, 9136 bp, artificial sequence
  synthetic oligonucleotide
ggatcctgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggcggatccgtatacatcgatgccaccatggtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggaggcctctgagcccttcaagaatgtgtacctcctcccccagaccaaccaactcctgggactctacaccatcatcaggaacaagaacaccaccaggccagacttcatcttctacagtgacaggatcatcaggctcctggtggaggagggcctcaaccacctccctgtgcagaagcagattgtggagactgacaccaatgagaactttgagggagtgtctttcatgggcaagatttgtggggtgtccattgtgagggctggggagagcatggagcagggcctgagggactgttgcaggagtgtgaggattggcaagatcctgatccagagggatgaggagactgccctgcccaagctgttctatgagaagctccctgaagacatctctgagaggtatgtcttcctcctggaccccatgctggcaactggaggctctgcaatcatggccactgaggtgctcatcaagaggggagtcaagcctgagaggatctacttcctcaacctcatctgctcaaaggagggcattgagaagtaccatgctgccttccctgaagtgaggattgtcactggggctctggacaggggcctggatgagaacaagtacctggtccctggcctgggagactttggggacagatactactgtgtctaaacctgagctagctatcgatgggcccactagtgtcgacgctagctctagatgtacaaagtggtgctagcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaaagcgctcaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagttaggtttaaacacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagctggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcg

SEQ ID NO: 8 - DNA, 35 bp, artificial sequence
  synthetic oligonucleotide
aattttatcgggatcccgagctcttacgcgtgcta

SEQ ID NO: 9 - DNA, 37 bp, artificial sequence
  synthetic oligonucleotide
cgatgtatacggatcctttatatcgtcccacggagcg

SEQ ID NO: 10 - DNA, 33 bp, artificial sequence; identical to SEQ ID NO: 5
  synthetic oligonucleotide

SEQ ID NO: 11 - DNA, 38 bp, artificial sequence; identical to SEQ ID NO: 6
  synthetic oligonucleotide

SEQ ID NO: 12 - DNA, 8778 bp, artificial sequence
  synthetic oligonucleotide
ggatcccgagctcttacgcgtgctagcccgggctagcccggccagtgccaagttgagacacattccacacattccactgcaagcttgagacacattccacacattccactgcaagcttggccagtgccaagttgagacacattccacacattccactgcaagcttgagacacattccacacattccactgcaagcttctagagatctgcaggtcgaggtcgacggtatcgataagcttgggggtgggcgccggggggaccttaaagcctctgccccccaaggagcccttcccagacagccgccggcacccaccgctccgtgggacgatataaaggatccgtatacatcgatgccaccatggtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggaggcctctgagcccttcaagaatgtgtacctcctcccccagaccaaccaactcctgggactctacaccatcatcaggaacaagaacaccaccaggccagacttcatcttctacagtgacaggatcatcaggctcctggtggaggagggcctcaaccacctccctgtgcagaagcagattgtggagactgacaccaatgagaactttgagggagtgtctttcatgggcaagatttgtggggtgtccattgtgagggctggggagagcatggagcagggcctgagggactgttgcaggagtgtgaggattggcaagatcctgatccagagggatgaggagactgccctgcccaagctgttctatgagaagctccctgaagacatctctgagaggtatgtcttcctcctggaccccatgctggcaactggaggctctgcaatcatggccactgaggtgctcatcaagaggggagtcaagcctgagaggatctacttcctcaacctcatctgctcaaaggagggcattgagaagtaccatgctgccttccctgaagtgaggattgtcactggggctctggacaggggcctggatgagaacaagtacctggtccctggcctgggagactttggggacagatactactgtgtctaaacctgagctagctatcgatgggcccactagtgtcgacgctagctctagatgtacaaagtggtgctagcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaaagcgctcaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagttaggtttaaacacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagctggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcg

SEQ ID NO: 13 - DNA, 267 bp, artificial sequence; identical to SEQ ID NO: 2
  synthetic oligonucleotide

SEQ ID NO: 14 - DNA, 32 bp, artificial sequence; identical to SEQ ID NO: 3
  synthetic oligonucleotide

SEQ ID NO: 15 - DNA, 32 bp, artificial sequence; identical to SEQ ID NO: 4
  synthetic oligonucleotide

SEQ ID NO: 16 - DNA, 8778 bp, artificial sequence
  synthetic oligonucleotide
ggatcctgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggcggatccatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatgcatccatggcggccgcctcgagcatcaccatcaccatcactgattctccttacgcatctgtgcggtatttcacaccgcatgtactagtgtcgacgctagctctagatgtacaaagtggtgctagcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaaagcgctcaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagttaggtttaaacacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagctggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcg

SEQ ID NO: 17 - DNA, 35 bp, artificial sequence; identical to SEQ ID NO: 8
  synthetic oligonucleotide

SEQ ID NO: 18 - DNA, 37 bp, artificial sequence; identical to SEQ ID NO: 9
  synthetic oligonucleotide

SEQ ID NO: 19 - DNA, 8420 bp, artificial sequence
  synthetic oligonucleotide
ggatcccgagctcttacgcgtgctagcccgggctagcccggccagtgccaagttgagacacattccacacattccactgcaagcttgagacacattccacacattccactgcaagcttggccagtgccaagttgagacacattccacacattccactgcaagcttgagacacattccacacattccactgcaagcttctagagatctgcaggtcgaggtcgacggtatcgataagcttgggggtgggcgccggggggaccttaaagcctctgccccccaaggagcccttcccagacagccgccggcacccaccgctccgtgggacgatataaaggatccatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatgcatccatggcggccgcctcgagcatcaccatcaccatcactgattctccttacgcatctgtgcggtatttcacaccgcatgtactagtgtcgacgctagctctagatgtacaaagtggtgctagcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaaagcgctcaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagttaggtttaaacacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagctggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcg

(19) United States
(12) Patent Application Publication
(10) Pub. No.: US 2018/0298340 A1
(43) Pub. Date: Oct. 18, 2018

(54) SYSTEMS FOR DETECTING, MONITORING OR TREATING DISEASES OR CONDITIONS USING ENGINEERED CELLS AND METHODS FOR MAKING AND USING THEM

(71) Applicant: The Regents of the University of California, Oakland, CA (US)

(72) Inventors: Weian ZHAO, Irvine, CA (US); Mark A. ECKERT, Chicago, IL (US); Linan LIU, Irvine, CA (US); Jan ZIMAK, Irvine, CA (US); Dongku KANG, Irvine, CA (US); Egest J. PONE, Irvine, CA (US); Shirley X. ZHANG, Irvine, CA (US); Mengrou LU, Irvine, CA (US)

(21) Appl. No.: 15/568,762

(22) PCT Filed: Apr. 21, 2016
(86) PCT No.: PCT/US16/28675

§ 371 Date: Oct. 23, 2017

Related U.S. Application Data
(60) Provisional application No. 62/152,560, filed on Apr. 24, 2015.

(51) Int. Cl. C12N 5/0775; A61K 35/28; A01K 67/027; A61P 35/04; A61K 49/00; C12Q 1/6886; C12N 9/02; C12N 5/0789

(52) U.S. Cl. CPC C12N 5/0662; A61K 35/28; A01K 67/0275; A61P 35/04; A61K 49/0056; C12Q 1/6886; C12Q 2600/106; C12Y 114/14003; C12N 5/0647; A01K 2217/05; A01K 2227/105; A01K 2267/0331; C12N 2510/00; C12N 9/0071

(57) ABSTRACT

Provided are methods for detecting and treating disease states, including cancer, diabetes, fibrosis, and autoimmune diseases, by detecting increased mechanical modulus, or stiffness, or targeting tissues having increased mechanical modulus, or stiffness. Practicing these methods provides specific and localized detection assays and therapies for these disease states. Provided are mechano-responsive cell systems that can selectively detect and treat cancer metastases by targeting the unique biophysical and mechanical properties in the tumor microenvironment. Provided are methods for making mechano-sensitive CAR T cells by using mechano-responsive promoter logic. Provided are blood tests using engineered stem cells that express reporters after the cells home to a specific niche and secrete the reporter into the blood, which can be then be detected with a blood test. In alternative embodiments, provided are ultrasensitive detection platforms, able to detect target molecules or cells in blood with single-molecule or single-cell sensitivity.

### RELATED APPLICATIONS

This application claims the benefit of priority to U.S. Provisional Patent Application Ser. No. 62/152,560, filed Apr. 24, 2015. The aforementioned application is expressly incorporated herein by reference in its entirety and for all purposes.

### STATEMENT AS TO FEDERALLY SPONSORED RESEARCH

This invention was made with government support under grant numbers 1DP2CA195763-01, awarded by the National Institutes of Health (NIH), DHHS; and BC121644, awarded by DOD CDMRP. The government has certain rights in the invention.

### TECHNICAL FIELD

This invention generally relates to bioanalysis, and detection, screening and treatment methodologies. In particular, in alternative embodiments, provided are methods for detecting and treating disease states, including cancer, diabetes, fibrosis, and autoimmune diseases, by detecting increased mechanical modulus, or stiffness, or targeting tissues having increased mechanical modulus, or stiffness. Practicing these methods provides specific and localized detection assays and therapies for these disease states, including cancer, diabetes, fibrosis, and autoimmune diseases. In alternative embodiments, provided are mechano-responsive cell systems (MRCS) that can selectively detect and treat cancer metastases and fibrotic-related diseases by targeting the unique biophysical and mechanical properties in a tumor or a fibrotic microenvironment. In alternative embodiments, provided are blood tests using mesenchymal stem cells (MSC) engineered to express reporters for detecting tumors and metastases. In alternative embodiments, provided are blood tests using engineered cells, e.g., stem cells, that express reporters, wherein after the cells home to a specific niche (e.g., tumor niche) they secrete the reporter into the blood, which can be then be detected with a blood test. In alternative embodiments, provided are ultrasensitive detection platforms, e.g., so-called Integrated Comprehensive Digital Droplet Detection (IC 3D), able to detect target molecules or cells in blood with single-molecule or single-cell sensitivity. In alternative embodiments, provided are engineered or recombinant T cells that express chimeric antigen receptors (CARs) to target antigens expressed on tumor cells and treat cancer metastases with selective mechano-responsive activation in the presence of both tumor antigens and the biophysical and mechanical properties in the tumor microenvironment. In alternative embodiments, provided are non-human transgenic animals engineered such that varying strength of mechano-signals can be detected by an array of mechano-sensitive promoters with different reporters, including fluorescent proteins.

### BACKGROUND

Many disease states in the body, including but not limited to cancer, diabetes, fibrosis, and autoimmune diseases, are difficult to detect especially at an early stage and even harder to treat using conventional methods.

Current detection methods include imaging modalities such as positron emission tomography (PET), computed tomography (CT), and magnetic resonance imaging (MRI), and biological tests such as histology, polymerase chain reaction (PCR), flow cytometry and enzyme-linked immunoassay (ELISA). These have downsides such as limited sensitivity and specificity, lengthy detection times, the use of irritating or harmful contrast agents and ionizing radiation, limited to no functionality in vivo, invasive procedures required for tissue biopsies and the need for extensive sample preparation or modification.

Current treatment methods such as systemic pharmacologic agents (e.g., chemotherapy) have limited targeting and specificity, limited effectiveness, and harmful side effects.

Another hurdle in cell therapy is the lack of tools and methods to monitor and manipulate the fate of transplanted cells including biodistribution, homing and engraftment, proliferation, differentiation, cell signaling, therapeutic efficacy and potential toxicity.

A major challenge in the field of detection and targeted treatment is finding appropriate biomarkers to indicate the diseased state. Unfortunately, molecular biomarkers are generally unreliable due to heterogeneity between patients.

### SUMMARY

In alternative embodiments, provided are engineered or recombinant cells, multiplexed systems or devices, and methods of using them, for cell engineering to target, to detect or monitor, or to treat or ameliorate abnormal cells or diseased tissues such as cancer. In alternative embodiments, provided are engineered or recombinant cells, or a multiplexed system or a device comprising, incorporating or using the engineered cell or recombinant, or a method or use of the engineered or recombinant cell, multiplexed system or device thereof, for cell engineering to target, to detect or monitor, or to treat abnormal cells or tissue of diseases, comprising:

(1) (a) providing a cell, or engineering method that changes the content of the cell to generate the engineered cell, and modifying the cell to comprise, include or have contained therein, or have the ability to express: a therapeutic agent, a converter enzyme, a pro-enzyme, an antibody, an exogenous protein, an exogenous nanoparticle, a homing agent, or any molecule or device that originally does not exist in the cell, or

(b) (the engineered or recombinant cell) comprising, includes or has contained therein, or is modified to have the ability to express: a therapeutic agent, a converter enzyme, a pro-enzyme, an antibody, an exogenous protein, an exogenous nanoparticle, a homing agent, or any molecule or device that originally does not exist in the cell,

wherein optionally the engineered or recombinant cell is an immune cell (optionally a T cell), mesenchymal stem cells (MSC), neural stem cells (NSC), hematopoietic stem cells (HSC) or a microorganism, optionally a bacteria,

and optionally the cell is engineered to comprise at least one exogenous nucleic acid having the ability to express: a therapeutic agent, a converter enzyme, a pro-enzyme, an antibody, an exogenous protein, an exogenous nanoparticle, a homing agent, or any molecule that originally does not exist in the cell, and expression of the nucleic acid is under the control of (operably linked to) a mechano-responsive promoter (wherein the promoter is activated by an increase in the stiffness of the cell's environment, or contact with a tissue or environment having an increased mechanical modulus, or stiffness), and optionally the mechano-responsive promoter comprises or is a YAP/TAZ mechanoresponsive promoter,

and optionally the engineered or recombinant cell comprises a homing agent, or is engineered to comprise an exogenous homing agent, comprising a protein or any form of molecule that facilitates or enhances the migration of the engineered or recombinant cell to certain or desired niche, including but not limited to a tumor niche, and optionally the homing agent is encoded by a nucleic acid under the control of (operably linked to) a mechano-responsive promoter, optionally a YAP/TAZ mechanoresponsive promoter,

and optionally the engineered or recombinant cell comprises a therapeutic agent, or is engineered to comprise an exogenous therapeutic agent, optionally a direct therapeutic agent, comprising a protein enzyme or any form of molecule that has a direct toxic or beneficial effect to other cells, and optionally the therapeutic agent is encoded by a nucleic acid under the control of (operably linked to) a mechano-responsive promoter, optionally a YAP/TAZ mechanoresponsive promoter,

and optionally the engineered or recombinant cell comprises a converter enzyme, or is engineered to comprise an exogenous converter enzyme, comprising a protein enzyme or any form of molecule that is capable of converting a toxic, inactive, or ineffective molecule into a diagnostic or therapeutic agent, and optionally the converter enzyme is encoded by a nucleic acid under the control of (operably linked to) a mechano-responsive promoter, optionally a YAP/TAZ mechanoresponsive promoter,

and optionally the engineered or recombinant cell comprises a pro-enzyme, or is engineered to comprise an exogenous pro-enzyme, comprising a protein enzyme or any form of molecule that is capable of being converted into a direct therapeutic agent, and optionally the pro-enzyme is encoded by a nucleic acid under the control of (operably linked to) a mechano-responsive promoter, optionally a YAP/TAZ mechanoresponsive promoter,

and optionally the engineered or recombinant cell comprises an antibody or antigen binding agent, or is engineered to comprise an exogenous antibody or antigen binding agent, wherein the antibody or antigen binding agent comprises a protein antibody or any form of molecule that is capable of binding to specific target, and optionally the antibody or antigen binding agent is encoded by a nucleic acid under the control of (operably linked to) a mechano-responsive promoter, optionally a YAP/TAZ mechanoresponsive promoter,

and optionally the engineered or recombinant cell comprises an exogenous protein that is originated from a species other than the engineered cell, or is modified from the natural form of the protein, and the exogenous protein is encoded by a nucleic acid under the control of (operably linked to) a mechano-responsive promoter, optionally a YAP/TAZ mechanoresponsive promoter,

and optionally the engineered or recombinant cell comprises an exogenous device, optionally a nanoparticle or comprising any molecule that the original cell does not possess,

and optionally the mechano-responsive promoter, optionally a YAP/TAZ mechanoresponsive promoter is engineered into the cell to drive an endogenous nucleic acid of interest, and optionally the mechanoresponsive promoter is engineered into the cell using CRISPR/Cas9 or equivalent methodology; or

(2) (a) providing an engineered or recombinant cell having a modified cellular content or comprising an exogenous factor to modify the cell's physiology, or biochemical or biophysical mechanisms, for differentiation, homing, mechano-signals, cell-cell communication, soluble factors, extracellular environment, or response to other factors, or

(b) (the engineered or recombinant cell) comprising, includes or has contained therein a modified cellular content or comprising an exogenous factor to modify the cell's physiology, or biochemical or biophysical mechanisms, for differentiation, homing, mechano-signals, cell-cell communication, soluble factors, extracellular environment, or response to other factors,

wherein optionally the exogenous factor to modify the cell's physiology, or biochemical or biophysical mechanisms, for differentiation, homing, mechano-signals, cell-cell communication, soluble factors, extracellular environment, or response to other factors is encoded by a nucleic acid under the control of (operably linked to) a mechano-responsive promoter, optionally a YAP/TAZ mechanoresponsive promoter,

and optionally the modified mechanism of differentiation of the engineered or recombinant cell alters its location and cellular content upon changing the cellular type specificity from low to high,

and optionally the modified mechano-signaling of the engineered or recombinant cell alters its location and cellular content upon receiving a stiffness and/or crosslinking signal from extracellular matrix or extracellular environment,

and optionally the modified mechanism for homing of the engineered or recombinant cell comprises homing to certain niche,

and optionally the modified mechanism of cell-cell communication of the engineered cell alters its location and cellular content upon interacting with other cells,

and optionally the modified generation of soluble factors by the engineered or recombinant cell alters its location and cellular content upon receiving factors in the extracellular environment,

and optionally the modified extracellular environment of the engineered or recombinant cell alters its location and cellular content in response to the content in the extracellular environment,

and optionally the modified chemical condition of the engineered or recombinant cell alters the location and/or cellular content of the engineered cell, wherein optionally the modified engineered cell comprises proteins, nucleic acids, lipids, carbohydrates, small molecules, pH, temperature, radiation, or any other factor for altering the location of the cell; or

(3) (a) providing an engineered or recombinant cell as set forth in steps (1) or (2) above, wherein the cell is modified to have, comprise or contain therein at least one exogenous nucleic acid having the ability to express: a therapeutic agent, a converter enzyme, a pro-enzyme, an antibody, an exogenous protein, an exogenous nanoparticle, a homing agent, or any molecule that originally does not exist in the cell, and expression of the nucleic acid is constitutive or activatable (inducible), or

(b) an engineered or recombinant cell as set forth in steps (1) or (2) above, wherein the engineered cell is modified to have, comprise or contain therein at least one exogenous nucleic acid having the ability to express: a therapeutic agent, a converter enzyme, a pro-enzyme, an antibody, an exogenous protein, an exogenous nanoparticle, a homing agent, or any molecule that originally does not exist in the cell, and expression of the nucleic acid is constitutive or activatable (inducible),

wherein optionally the exogenous nucleic acid under the control of (operably linked to) a mechano-responsive promoter, optionally a YAP/TAZ mechanoresponsive promoter,

wherein optionally the constitutive expression persists regardless of the extracellular environment.

wherein optionally the activatable or inducible expression begins upon a mechanism described in (2), or is activatable or inducible by expression of an exogenous factor to modify the cell's physiology, or a biochemical or biophysical mechanism, or expression of a factor for differentiation, homing, mechano-signaling, cell-cell communication, exposure to a soluble factor or an extracellular environment, or response to other factors,

wherein optionally the cell engineering is by a method comprising a genetic method, optionally CRISPR/Cas9 method or equivalent, or a non-genetic method; or

(4) (a) providing an engineered or recombinant cell that enables treatment of a disease or condition through the expression of a converter enzyme, a direct therapeutic enzyme, a pro-enzyme, an antibody, or any molecule that directly or indirectly aids in the therapeutic process, or

(b) (the engineered or recombinant cell) comprising, includes or has contained therein a converter enzyme, a therapeutic enzyme, a pro-enzyme, an antibody, or any molecule that directly or indirectly aids in the therapeutic process,

wherein optionally the converter enzyme, therapeutic enzyme, pro-enzyme, antibody, or molecule that directly or indirectly aids in the therapeutic process is encoded by an exogenous or an endogenous nucleic acid under the control of (operably linked to) a mechano-responsive promoter, optionally a YAP/TAZ mechanoresponsive promoter, and optionally the endogenous nucleic acid is engineered to be operably linked to a mechano-responsive promoter by a CRISPR/Cas9 methodology or equivalent,

wherein optionally the treatment comprises use of a converter enzyme or any protein or any other molecule that converts an inactive form of therapeutic agent into its active form,

and optionally the treatment comprises use of a direct therapeutic enzyme that directs alteration of the content of a cell or an extracellular environment,

and optionally the treatment comprises use of a pro-enzyme or any protein or any molecule produced by the engineered cell, wherein its form is altered from inactive to active in response to mechanisms described in (2), and delivers a therapeutic effect in its active form,

and optionally the treatment comprises use of an antibody or immunoglobulin produced by the engineered cell, which aids in the therapeutic process directly or indirectly; or

(5) (a) providing an engineered or recombinant cell that enables an assay for detection or diagnostics, companion diagnostics, or scientific and research tools, or

(b) (the engineered cell) comprising a nucleic acid encoding a protein that enables detection of the cell, or enables detection of the cell when the cell is exposed to a new environment, optionally a tissue or environment having an increased mechanical modulus, or stiffness, optionally the nucleic acid is under the control of (operably linked to) a mechano-responsive promoter, optionally a YAP/TAZ mechanoresponsive promoter,

wherein optionally the utility, assay for detection or diagnostics comprises of in vitro, in vivo, ex vivo, in situ or any other form of assay that enables the detection of the cellular location and/or content of the engineered cell,

and optionally the utility, companion diagnostics comprises of equipment and/or platform that enables the detection of cellular location and/or content of the engineered cells,

and optionally the utility, companion diagnostics comprises of equipment and/or platform that enable cell fate tracking and monitoring by detecting probes (e.g., enzymes) secreted by the cell into biological fluids including e.g., blood and urine,

and optionally the probes can be the therapeutic itself (e.g., a gene or a protein) in the case of gene cell therapy or other molecules or agents engineered into the cell,

and optionally the utility, companion diagnostics comprises of equipment and/or platform that permits single molecule detection from biological samples,

and optionally the utility, scientific and/or research tools comprise of the usage of the engineered cell that facilitate the scientific study of biological processes; or

(6) (a) providing an engineered or recombinant cell that enables monitoring for post cellular gene therapy and tracking for safety through the expression of exogenous molecules, or

(b) (the engineered or recombinant cell) comprising a nucleic acid encoding a protein that enables monitoring for post cellular gene therapy and tracking for safety through the expression of exogenous molecules, and optionally the nucleic acid is under the control of (operably linked to) a mechano-responsive promoter, optionally a YAP/TAZ mechanoresponsive promoter; or

(7) (a) providing an engineered or recombinant cell that directly or indirectly aids in treating or ameliorating a cancer, a cancer metastases, a tissue fibrosis, cell fate tracking, diabetes, wound healing, cosmetics, osteoporosis, regenerative medicine, or an immune disease,

(b) (the engineered or recombinant cell) comprising a nucleic acid encoding a protein that treats or ameliorates a cancer, a cancer metastases, a tissue fibrosis, cell fate tracking, diabetes, wound healing, cosmetics, osteoporosis, regenerative medicine, or an immune disease, and optionally the nucleic acid is under the control of (operably linked to) a mechano-responsive promoter, optionally a YAP/TAZ mechanoresponsive promoter,

wherein optionally the cancer or cancer metastases comprises a condition when cancer spreads into tissue other than its origination, and the tissue other than its origination has a sufficient mechanical modulus, or stiffness to activate (turn on) the mechano-responsive promoter,

and optionally the tissue fibrosis comprises a condition of excessive formation of fibrous connective tissue, and optionally the excessive formation of fibrous connective tissue has a sufficient mechanical modulus, or stiffness to activate (turn on) the mechano-responsive promoter,

and optionally the cell fate tracking comprises a method of detecting the fate of engineered cell in vivo,

and optionally the diabetes comprises prolonged high level of blood glucose,

and optionally the wound healing comprises regeneration and remodeling of damaged tissue,

and optionally the cosmetics comprises improving appearance of the body,

and optionally the osteoporosis comprises a decreased bone mass and density,

and optionally the regenerative medicine comprises a process of replacing, engineering or regenerating human cells, tissues or organs to restore or establish normal function,

and optionally the immune disease comprises of a disease caused by a deficient or malfunctioned immune system.

In alternative embodiments, provided are engineered or recombinant cells for use in treating, ameliorating, preventing or removing a scar tissue, wherein the cells comprise:

(a) an exogenous nucleic acid encoding a secreted enzyme capable of disrupting or removing a scar tissue,

wherein expression of the exogenous nucleic acid is under the control of (operably linked to) a mechano-responsive promoter (wherein the promoter is activated by an increase in the stiffness of the cell's environment, or contact with a tissue or environment having an increased mechanical modulus, or stiffness), and optionally the mechano-responsive promoter comprises or is a YAP/TAZ mechanoresponsive promoter, or

(b) an endogenous nucleic acid encoding a secreted enzyme capable of disrupting or removing a scar tissue, wherein the endogenous nucleic acid is engineered to be operably linked to a mechano-responsive promoter, optionally by use of a CRISPR/Cas9 methodology or equivalent, or homologous recombination,

wherein optionally the engineered or recombinant cell is capable of targeting or binding to a fibrosis or a scar tissue, or is engineered to target or bind to a fibrosis or a scar tissue,

and optionally the engineered or recombinant cell is a stem cell, a fibroblast, an epithelial cell, or an immune cell, optionally a T cell, a lymphocyte or a megakaryocyte.

In alternative embodiments, provided are methods for treating, ameliorating, dissolving, preventing or removing a scar tissue or a fibrosis in an individual in need thereof, or

use of an engineered or recombinant cell for treating, ameliorating, preventing or removing a scar tissue a fibrosis, or

an engineered or recombinant cell for treating, ameliorating, preventing or removing a scar tissue a fibrosis, comprising:

(a) providing an engineered or recombinant cell as provided herein, and

(b) administering the cell to the individual in need thereof.

wherein optionally the fibrosis or scar treated, ameliorated, dissolved, prevented or removed comprises a fibrosis or scar associated with a fibrosis-related disease, optionally a lung, liver, kidney, heart or vessel fibrosis, or a wound-induced or surgical induced scar, or a scar induced by a myocardial infarction or a myocardial infection.

In alternative embodiments, provided are engineered or recombinant cells for use in treating, ameliorating or preventing a condition responsive to an antibody or a chimeric antigen receptor (CAR), wherein the cell comprises:

(a) an exogenous nucleic acid encoding an antibody a chimeric antigen receptor (CAR), wherein the antibody or CAR can treat, ameliorate or prevent a condition responsive to an antibody or a chimeric antigen receptor (CAR),

wherein expression of the exogenous nucleic acid is under the control of (operably linked to) a mechano-responsive promoter (wherein the promoter is activated by an increase in the stiffness of the cell's environment, or contact with a tissue or environment having an increased mechanical modulus, or stiffness), and optionally the mechano-responsive promoter comprises or is a YAP/TAZ mechanoresponsive promoter, or

(b) an endogenous nucleic acid encoding an antibody, wherein the endogenous nucleic acid is engineered to be operably linked to a mechano-responsive promoter, optionally by use of a CRISPR/Cas9 methodology or equivalent, or homologous recombination,

wherein optionally the engineered or recombinant cell is capable of targeting or binding to a specific or a desired cell, organ or tissue, or is engineered to target or bind to a specific or a desired cell, organ or tissue, optionally the engineered or recombinant cell is capable of targeting or binding to a cancer or tumor, optionally a solid tumor, or a cancer metastasis,

and optionally the engineered or recombinant cell is a stem cell, a fibroblast, an epithelial cell, or an immune cell, optionally a T cell, a lymphocyte or a megakaryocyte.

In alternative embodiments, provided are methods for treating, ameliorating or preventing a condition responsive to an antibody or a chimeric antigen receptor (CAR) in an individual in need thereof, or

use of an engineered or recombinant cell for treating, ameliorating or preventing a condition responsive to an antibody or a chimeric antigen receptor (CAR), or

an engineered or recombinant cell for treating, ameliorating or preventing a condition responsive to an antibody or a chimeric antigen receptor (CAR), comprising:

(a) providing an engineered or recombinant cell as provided herein, and

(b) administering the cell to the individual in need thereof,

wherein optionally the condition responsive to an antibody or a chimeric antigen receptor (CAR) is a cancer or tumor, optionally a solid tumor, or a cancer metastasis.

In alternative embodiments, provided are engineered or recombinant cells for use in delivering a detectable probe or molecule, or a therapeutic molecule, to a targeted cell, organ or tissue in an individual in need thereof, wherein the cell comprises:

(a) an exogenous nucleic acid encoding a detectable probe or molecule, or a therapeutic molecule,

wherein expression of the exogenous nucleic acid is under the control of (operably linked to) a mechano-responsive promoter (wherein the promoter is activated by an increase in the stiffness of the cell's environment, or contact with a tissue or environment having an increased mechanical modulus, or stiffness), and optionally the mechano-responsive promoter comprises or is a YAP/TAZ mechanoresponsive promoter, or

(b) an endogenous nucleic acid encoding a therapeutic molecule or a detectable molecule, wherein the endogenous nucleic acid is engineered to be operably linked to a mechano-responsive promoter, optionally by use of a CRISPR/Cas9 methodology or equivalent, or homologous recombination,

wherein optionally the engineered or recombinant cell is capable of targeting or binding to a specific or a desired cell, organ or tissue, or is engineered to target or bind to a specific or a desired cell, organ or tissue, optionally the engineered or recombinant cell is capable of targeting or binding to a cancer or tumor, optionally a solid tumor, or a cancer metastasis,

wherein optionally the detectable probe or molecule comprises a fluorescent protein, optionally an enhanced green fluorescent protein (eGFP), a beta-galactosidase (beta-gal) (optionally an E. coli beta-gal), a horseradish peroxidase (HRP) or a luciferase, and optionally the therapeutic molecule comprises a cytosine deaminase (CD),

and optionally the detectable probe or molecule is a secreted detectable probe or molecule, and optionally after secretion by the cell the detectable probe or molecule is detectable in a body fluid, optionally blood or urine,

and optionally the engineered or recombinant cell is a stem cell, a fibroblast, an epithelial cell, or an immune cell, optionally a T cell, a lymphocyte or a megakaryocyte.

In alternative embodiments, provided are methods for delivering a detectable probe or a therapeutic molecule to a targeted cell, organ or tissue in an individual in need thereof, or

use of a detectable probe or a therapeutic molecule for detecting or treating a targeted cell, organ or tissue in an individual in need thereof, or

an engineered or recombinant cell for detecting or treating a targeted cell, organ or tissue in an individual in need thereof, comprising:

(a) providing an engineered or recombinant cell as provided herein, and

(b) administering the cell to the individual in need thereof,

wherein optionally a cancer or tumor, optionally a solid tumor, or a cancer metastasis, is treated or detected by the detectable probe or the therapeutic molecule.

In alternative embodiments, provided are non-human transgenic animals comprising an engineered or recombinant cell as provided herein.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

FIG. 1 schematically illustrates an overview of exemplary embodiments as provided herein: exemplary methods of monitoring and manipulating the fate of transplanted cells. Provided are systems that employ engineered (e.g., genetically modified or recombinant) cells that are able to target, detect abnormal cells or tissues of disease states. In addition, the transplanted cells are able to respond to cellular or niche characteristics including biochemical or physical markers to produce, for examples, reporter molecules for imaging and diagnostic purposes or therapeutics to treat a disease. This will allow for earlier and more accurate diagnosis as well as post treatment monitoring and targeted therapeutic delivery and treatment. Provided are platform technologies to track and monitor transplanted cells from hours to years in vivo. Instead of in situ imaging that is used currently, provided are methods which measure secreted probes in biological fluids such as blood or urine, wherein the secretion is coupled to a particular cellular function.

FIG. 2 schematically illustrates exemplary approaches for cell engineering and expression. In alternative embodiments, the gene, marker, therapeutic, or probe of interest inserted into cells are carried by delivery vehicles or with non-genetic methods. The delivery vehicle can be genome editing tools (e.g., CRISPR/Cas^{[1]}) or other delivery vehicle. To engineer the cell with insertions, the delivery vehicles can be transfected, viral transduced, or through other delivery methods.

FIG. 3 schematically illustrates and lists a few examples of cell types used to practice embodiments as provided herein. In alternative embodiments, many types of cells are engineered to selectively activate promoters and gene expression in response to certain microenvironments. This will allow for selection of cells best suited for specific targeting, detection, or therapeutic delivery. For example, in alternative embodiments mesenchymal stem cells are used as then can be ideal for tumor detection and delivery of cancer drugs since they exhibit natural tumor tropism.

FIG. 4 schematically illustrates an exemplary method for genetic editing strategy. In alternative embodiments, promoters of genes responsive to specific ranges of stiffness are cloned from genomic DNA (left) or constructs (right) and subcloned into promoterless vectors to drive expression of enhanced green fluorescent protein (eGFP), luciferase (reporter), cytosine deaminase (therapeutics), etc. In alternative embodiments, the constructs are permanently transduced into cells such as mesenchymal stem cells (MSC) to produce stable engineered MSC cell lines.

FIG. 5A, FIG. 5B, FIG. 5C and FIG. 5D illustrate exemplary mechanisms to engineer cells to post or react to certain function. In alternative embodiments, engineered cells can address diagnostic or therapeutic problems using several main mechanisms. (FIG. 5A) Homing: Cells can be engineered to enhance the homing ability to certain niche (e.g., inflammation). Also the promoters of homing factors (e.g., SDF-1) can be used to drive reporter or therapeutics by specifically targeting diseased microenvironments. (FIG. 5B) Stiffness: Cells can be engineered to detect specific ranges of tissue stiffness and selectively activate promoters for gene expression. For example, an engineered cell (green) can be activated once it attaches to stiffer, crosslinked collagen (red crosshatching). Cells that are not exposed to this stiffer microenvironment will remain inactivated (gray). (FIG. 5C) Differentiation: Engineered stem cells can undergo differentiation in response to their environment to potentially integrate into and regenerate damaged tissues. (FIG. 5D) Soluble factors: Engineered cells can secrete useful soluble factors that can then drive useful downstream signal pathways.

FIG. 6A-D illustrate and list a few examples of problems that are solved using embodiments provided herein, including (FIG. 6A) cancer metastases, (FIG. 6B) tissue fibrosis/scar dissolving (fibrosis-related diseases, including lung, liver, kidney, heart & vessels), (FIG. 6C) cell fate tracking (e.g., post HSC transplantation), (FIG. 6D) cardiovascular diseases including atherosclerosis, diabetes (e.g., beta-cell stiffness-directed regeneration), wound healing (e.g., diabetic ulcer), cosmetics (e.g., wrinkles remover, to regenerate fatty tissue and renew skin cells: an anti-aging treatment), osteoporosis (or any types of bone/muscle regeneration), regenerative medicine, and immune diseases.

FIG. 7 schematically illustrates a general overview of exemplary methods for monitoring engineered cells in vivo by detecting their secreted markers using single molecule in vitro detection assays. In alternative embodiments, cells are engineered with secreted reporter enzymes constitutively or after specific promoter (e.g., YAP (Yes-associated protein) and TAZ (transcriptional coactivator with PDZ-binding motif, also known as WWTR1) for stiffness sensing). In alternative embodiments, after transplanting the engineered cells into animals or patients, the reporter enzymes will be expressed after the cells home to specific niche (e.g., tumor niche) and secrete the enzymes into blood/urine, which can be detected with a blood/urine test. In alternative embodiments, the blood/urine test utilize ultrasensitive detection methods including e.g., integrated comprehensive digital droplet detection (IC 3D). In IC 3D, the blood/urine sample is compartmentalized into picoliter-size droplets in oil, containing one or no enzyme in each droplet, and the droplets containing reporter enzymes will react with their specific fluorogenic substrate. The fluorescent droplet can be detected with 3D particle counter.

FIG. 8 schematically illustrates an exemplary ultrasensitive Integrated Comprehensive Digital Droplet Detection (IC 3D) system used to practice alternative embodiments. In alternative embodiments, the reagents (e.g., blood sample containing targets, and sensors for targets) are mixed in oil, generating picoliter-size water-in-oil droplets that either contains one or no target. Within the droplet containing target, the sensor and targets will react and generate fluorescent product. The fluorescent droplet can be detected with 3D particle counter to determine the number of fluorescent droplet, which correspond to the number of target contained in the original sample.

FIG. 9 is a list of exemplary bioassays for single molecule detection that can be used to analyze secreted probes in biological samples to monitor the fate of transplanted cells in vivo, and also lists exemplary probes that can be used to engineer cells for the single molecule detection assay.

FIG. 10 schematically illustrates an exemplary reaction for an enzyme microfluidics assay. In alternative embodiments, each picoliter droplet or micro well contains either one or no target (enzyme). With the presence of target, the fluorogenic substrate are turned into fluorescent product, resulting in a fluorescent droplet or micro well.

FIG. 11 schematically illustrates an exemplary reaction for a nucleic acid microfluidics assay. In alternative embodiments, each picoliter droplet or micro well contains either one or no target DNA or RNA. With the presence of target DNA or RNA, PCR, Reverse transcription PCR (RT-PCR), Real-time PCR or Taqman PCR is triggered, resulting in a fluorescent droplet or micro well.

FIG. 12A and FIG. 12B, and FIG. 13C and FIG. 13D, schematically illustrate examples of targeted delivery of therapeutics for treatment. (FIG. 12A) Converter enzyme: Cells can be engineered to express a converter enzyme such as cytosine deaminase (CD), which can then convert an inactive prodrug into an active drug after it has been administered. (FIG. 12B) Direct therapeutic enzyme: Cells can be engineered to directly express a therapeutic enzyme, such as matrix metalloproteinases (MMPs) to treat tissue fibrosis. (FIG. 13C) Pro-enzyme: e.g., pro-caspase. (FIG. 13D) Antibodies: e.g., Trastuzumab, which is an anti-cancer drug.

FIG. 14 schematically illustrates an overview of exemplary methods for engineering mesenchymal stem cells (MSC) to create a mechano-responsive cell system (MRCS) for detection and treatment of breast cancer metastases in the lung. In alternative embodiments, the MRCS are activated by specific ranges of stiffness linked to collagen crosslinking found at the metastatic niche (red crosshatching). The tumor-homing MRCS can be used to detect the metastatic niche through fluorescent or bioluminescent reporters (left side), or to locally activate therapeutics specifically at the metastatic niche (right side). In alternative embodiments, Mechano-responsive stem cell system (MRCS) is used to elucidate complex mechanobiology in vivo, and to selectively detect and treat cancer metastases by targeting the unique biophysical cues in the tumor niche.

FIG. 15A-P illustrate images of exemplary mechano-responsive cell systems (MRCSs) that can sense stiffness in vitro and turn on eGFP signal on stiff substrate. MRCS was plated on (FIG. 15A, FIG. 15E, FIG. 15I, FIG. 15M) soft (approximately 1 kPa), (FIG. 15B, FIG. 15F, FIG. 15J, FIG. 15N) medium (approximately 10 kPa), (FIG. 15C, FIG. 15G, FIG. 15K, FIG. 15O) firm (approximately 40 kPa) poly-acrylamide and (FIG. 15D, FIG. 15H, FIG. 15L, FIG. 15P) glass. Note that eGFP was turned on responding to high stiffness (greater than 10 kPa), suggesting that this MSC-based reporter is specific for stiffness. (FIG. 15E-H) YAP/TAZ (red) relocalization is also regulated by stiffness. (FIG. 15A-D) eGFP (green) and (I-L) 4′, 6-diamidino-2-phenylindole (DAPI, blue, nuclear counterstain) are displayed. Scale bar=50 μm.

FIG. 16A-L illustrate images of exemplary MRCS-eGFP showing that MRCS is stiffness specific in vitro. MRCS was plated on firm (approximately 40 kPa) poly-acrylamide and treated with (FIG. 16A, FIG. 16D, FIG. 16G, FIG. 16J) 5004 blebbistatin and (FIG. 16B, E, FIG. 16H, FIG. 16K) 10 μM ML-7 (myosin light-chain kinase inhibitors) as well as (FIG. 16C, FIG. 16F, FIG. 16I, FIG. 16K) 20 μM PF228 (focal adhesion kinase inhibitor). Note that (FIG. 16A-C) eGFP (green) was turned off and (FIG. 16D-F) YAP/TAZ (red) was in cytoplasm, suggesting that MRCS sensing is reversibly stiffness-dependent. (FIG. 16G-I) DAPI (blue, nuclear counterstain) are displayed. Scale bar=50 μm.

FIG. 17 graphically illustrates data showing that MRCS-eGFP is stiffness specific in vitro with real-time RT-PCR analysis in MRCS on polyacrylamide hydrogels. Expression of eGFP (green) and YAP/TAZ downstream factors (CTGF, cyan and ANKRDI, purple) was increased on stiff substrate and was downregulated on soft substrate or with mechano-sensing inhibitors. It shows that MRCS can be regulated depending on stiffness. **P<0.01.

FIG. 18 graphically illustrates data showing that MRCS-luciferase (MRCS-Luc) is stiffness specific in vitro. MRCS-Luc was seeded on substrates with different stiffness. It shows that luciferase activity was unregulated on stiff substrate and downregulated on soft substrate or with mechano-sensing inhibitors, indicating MRCS is stiffness-dependent. D-Luciferin: 150 μg/ml in MSC growth medium. *P<0.05, **P<0.01.

FIG. 19A-P illustrate images showing that MRCS-CD is stiffness-responsive in vitro. MRCS was plated on (FIG. 19A, FIG. 19E, FIG. 19I, FIG. 19M) soft (approximately 1 kPa), (FIG. 19B, FIG. 19F, FIG. 19J, FIG. 19N) medium (approximately 10 kPa), (FIG. 19C, FIG. 19G, FIG. 19K, FIG. 19O) firm (approximately 40 kPa) poly-acrylamide and (FIG. 19D, FIG. 19H, FIG. 19L, FIG. 19P) glass. Note that cytosine deaminase (CD) was turned on responding to high stiffness (greater than 10 kPa), suggesting that this MSC-based reporter is specific for stiffness. (FIG. 19E-H) YAP/TAZ (red) relocalization is also regulated by stiffness. (FIG. 19A-D) CD (green) and (FIG. 19I-L) DAPI (blue, nuclear counterstain) are displayed. Scale bar=25 μm.

FIG. 20 graphically illustrates data showing that MRCS-CD is stiffness specific in vitro based on stiffness inhibitor studies. MRCS was plated on firm (approximately 40 kPa) poly-acrylamide and treated with (A,D,G,J) 50 μM blebbistatin and (B,E,H,K) 10 μM ML-7 (myosin light-chain kinase inhibitors) as well as (C,F,I,K) 20 μM PF228 (focal adhesion kinase inhibitor). Note that (FIG. 20A-C) cytosine deaminase (CD) (green) was turned off and (FIG. 20D-F) YAP/TAZ (red) was in cytoplasm, suggesting that MRCS sensing is reversibly stiffness-dependent. (G-I) DAPI (blue, nuclear counterstain) are displayed. Scale bar=50 μm.

FIG. 21 graphically illustrates data showing that MRCS-CD kills cancer cells in response to stiffness with 5-Fluorocytosine (5-FC) in vitro. MRCS-CD was co-cultured with luciferase MDA-MB-231 breast cancer cells (231:MRCS=2:1) with (800 μg/ml) or without 5-FC on different stiffness: total cell proliferation (2,3-Bis-(2-Methoxy-4-Nitro-5-Sulfophenyl)-2H-Tetrazolium-5-Carboxanilide, i.e., XTT assay) is displayed. All the data was normalized with breast cancer only (231:MRCS=1:0) on certain stiffness. **P<0.01, ***P<0.001.

FIG. 22 schematically illustrates a timeline of an exemplary animal experiment to test MRCS-CD with 5-FC in vivo.

FIG. 23 illustrates images of pictures of nude mice showing that MRCS-CD decreases lung metastasis signals in vivo. Representative pictures of nude mice received MRCS-CD treatments. MSC that constitutively expressing cytosine deaminase (CD) (upper left panel), MRCS-CD (lower left panel), native MSC (upper right panel) and phosphate-buffered saline (PBS) (lower right panel) were intravenously (i.v.) infused into nude mice 6 weeks after cancer seeding. Luciferase imaging was taken before (DO, left panels) and after (D9, middle panels) 5-FC treatment intraperitoneally (i.p.) as well as long-term outcome (6 weeks, right panels). D-Luciferin=150 mg/kg (i.p.). Color Scale: Min=9×10⁴; Max=3×10⁵. C-MSC: Constitutive positive control; N-MSC: Native MSC.

FIG. 24 graphically illustrates data showing MRCS-CD decreases lung metastasis signals in vivo (short term) with the quantification of luciferase signals that are proportional to cancer mass in the lung. Relative Metastasis Index (RMI)=Luciferase read on D9 (after)/Luciferase read on DO (before). n=9 for each group. ***P<0.001.

FIG. 25 graphically illustrates data showing MRCS-CD decreases lung metastasis signals in vivo (long term). Quantification of luciferase signals that are proportional to cancer mass in the lung before (week 0, red) and after (week 6) prodrug treatment. Lung Metastasis Index (LMI)=Log₁₀ [(Luciferase read of the tested mouse)/(Luciferase read of healthy mice average)], i.e., the LMI of healthy mice=0. n=9 for each group. *P<0.05 (C-MSC, week0 vs. week6), **P<0.01 (MRCS-CD, week0 vs. week6). The differences between “week 0” groups are not statistically significant.

FIG. 26 graphically illustrates data showing MRCS-CD increases mice survival. In this Kaplan-Meier survival curve for breast cancer (MDA-MB-231) lung metastasis nude mice treated with C-MSC (red), MRCS-CD (blue), N-MSC (green) and PBS (black) with 5-FC administration, MRCS-CD treated mice showed an increase in survival time compared to those in N-MSC or PBS groups. n=9 for each group.

FIG. 27A-E illustrate images of stained lung sections showing that MRCS-CD killing of cancer cells in vivo with minimal side effects. (FIG. 27A-E) Frozen sections of lungs of Luc-RFP-231 tumor-bearing and tumor-free nude mice sacrificed 24 hours following CD-MSC, MRCS-CD, N-MSC or DPBS infusion were stained with anti-Annexin V (green) and DAPI (blue). RFP signal (red) indicates the presence of lung metastasis. Scale bar=100 μm. CD-MSC: Constitutive positive control; N-MSC: Native MSC.

FIG. 28A-F illustrate images of pictures of lungs from tumor bearing nude mice showing that constitutively engineered MSC (C-MSC) cause lung tissue damages in vivo. Representative pictures of frozen section samples of lungs from tumor bearing nude mice treated by MSC that constitutively expressing cytosine deaminase (CD) (A,D), native MSC (FIG. 28B, FIG. 28E) and PBS (FIG. 28C, FIG. 28F) before (FIG. 28A-C) and after (FIG. 28D-F) 5-FC injections by Terminal deoxynucleotidyl transferase dUTP nick end-labeling (TUNEL) assays. These data indicate that MSC that constitutively expressing CD caused more lung tissue damages than native MSC and PBS. Horseradish peroxidase (HRP) signals (brown) stand for damaged nuclei and green signals are methyl green counterstain of normal nuclei. Scale bar=100 μm.

FIG. 29A-D illustrate images of pictures of tumor-bearing lungs showing that MRCS-CD causes minimal lung tissue damages in vivo with TUNEL assay. Representative pictures of frozen section samples of tumor bearing lungs (A, C) and healthy lungs (B,D) treated by MRCS-CD before (A,B) and after (C,D) 5-FC injections by TUNEL assays. These data indicate that MRCS-CD caused minimal lung tissue damages. HRP signals (brown) stand for damaged nuclei and green signals are methyl green counterstain of normal nuclei. Scale bar=100 μm.

FIG. 30 graphically illustrates data showing MRCS-CD causes minimal lung tissue damages in vivo with TUNEL assay. Representative quantification of TUNEL positive cells (%) before and after 5-FC treatment. n.s., not significant, *P<0.05, **P<0.01 and ****P<0.0001.

FIG. 31A-D illustrate images of pictures of tumor-bearing lungs showing with H&E staining that MRCS-CD causes minimal lung tissue damages in lungs. Representative pictures of frozen section samples of tumor bearing lungs treated by (A)C-MSC, (B) MRCS-CD, (C)N-MSC and (D) PBS after 5-FC injection by H&E assays. These data indicate that MRCS-CD caused minimal lung tissue damages. Scale bar=100 μm.

FIG. 32 schematically illustrates how MRCS treats lung metastasis. Lungs of tumor-bearing and tumor-free mice were harvested 6 weeks after cancer seeding and lungs of tumor bearing mice were harvested 2 weeks and 8 weeks after treatment of MRCS-CD. The lungs post-treatment of MRCS-CD and C-MSC groups had less tumor nodules than N-MSC and PBS groups.

FIG. 33 illustrates images of mice showing that MRCS shows minimal side effects (inflammation) in vivo. 2 weeks after MRCS-CD treatment, inflammation was observed in C-MSC treated groups but not in MRCS-CD treated ones.

FIG. 34 illustrates images of mice showing that MRCS shows minimal side effects (weigh loss) in vivo. 6 weeks after MRCS-CD treatment, weigh loss (skinny mice) was observed in C-MSC treated groups but not in MRCS-CD treated ones.

FIG. 35 schematically illustrates the timeline of an exemplary animal experiment to test MRCS-CD with 5-FC in vivo. 6 weeks after firefly luciferase and red fluorescent protein expressing MDA-MB-231 (Fluc-RFP-231) were seeded s.c. into the fat pad of nude mice, in vivo Fluc imaging (mice were injected i.p. with D-Luciferin (150 mg/kg in DPBS)) was performed to monitor the metastasis signals. 1×10⁶ engineered MSC were administered systemically into both tumor-free and tumor-bearing mice (Day 0). Then mice were injected i.p. with prodrug (5-FC, 500 mg/kg in DPBS) twice per day for 5 days (Day 2-6) and once per day for 2 days (Day 7-8). In vivo Fluc activity was measured on D 0 and Day 10 using an IVIS Lumina to begin data acquisition 10 minutes after substrate administration. One mouse per group was euthanized on Day 10 for ex vivo assays. Rest of the mice were kept for survival experiment.

FIG. 36 graphically illustrates data showing MRCS-CD reduces primary tumor size with prodrug in vivo. Mice with primary tumors in breast fat pad were treated with MRCS-CD and prodrug as described. Tumor size was measured every day since Day 0. The negative change in tumor volume of MRCS-CD and C-MSC groups was higher than that in N-MSC group starting from 6 days after treatment begins.

FIG. 37 graphically illustrates data (left panel) and illustrates images of mice (right panel) showing that MRCS-CD reduces primary tumor size with prodrug in vivo. Mice with primary tumors in breast fat pad were treated with MRCS-CD and prodrug as described. Tumor size was measured every day since Day 0. The negative change in tumor volume of MRCS-CD and C-MSC groups was higher than that in N-MSC group starting from 6 days after treatment begins.

FIG. 38 graphically illustrates data showing that (MRCS-CD reduces luciferase signals from primary tumor with prodrug in vivo. Mice with primary tumors in breast fat pad were treated with MRCS-CD and prodrug as described. Fluc activity was measured. The Fluc from primary tumors of MRCS-CD and C-MSC groups was reduced while that in N-MSC and PBS group increases.

FIG. 39A-C illustrates tissue section images showing MRCS-CD causes primary tumor tissue damages in vivo. Representative pictures of frozen section samples of subcutaneous (s.c.) MDA-MB-231 breast cancer primary tumors treated by (FIG. 39A)C-MSC, (FIG. 39B) MRCS-CD and (FIG. 39C)N-MSC in the presence of 5-FC. Mice were euthanized tissues were harvested on Day 10 as described. These data indicate that MRCS-CD caused tissue damages in primary tumors. HRP signals (brown) stand for damaged nuclei and green signals are methyl green counterstain of normal nuclei. Scale bar=100 μm.

FIG. 40 schematically illustrates exemplary uses of engineered mesenchymal stem cells (MSC) to detect cancer. In alternative embodiments, Engineered MSC (gray) secreting humanized Gaussia luciferase (hGluc, green) are systemically administrated into patients with cancer (breast cancer lung metastasis in this case). In alternative embodiments, Engineered MSC home to tumor (cyan) niche and persist, secreting hGluc into blood. Then patient blood can be collected and hGluc activity measured.

FIG. 41A-C graphically illustrate data, and FIG. 41B also illustrates an image of a serial dilution, showing that humanized Gaussia luciferase (hGluc) is secreted in vitro and is stable in blood. (FIG. 41A) Mesenchymal stem cells expressing humanized Gaussia luciferase (hGluc-MSC) and native MSC (N-MSC) were seeded onto 96 well plates. 24 hours later cell-free conditioned medium (CM) was harvested. The hGluc substrate coelenterazine (CTZ) was added with a final concentration of 20 μM. hGluc activity was measured immediately using a plate reader (Absorbance at wavelengths 300-700 nm, exposure time=2 s). (FIG. 41B) Serial dilution of hGluc-MSC CM was performed in PBS and CTZ was added at a final concentration of 20 μM. hGluc activity was measured with an IVIS Lumina (exposure time=0.5 s). Color scale: Min=6.64e8; Max=8.93e9. (FIG. 41C) CM of hGluc-MSC was harvested and incubated with human serum for 10 minutes, 2 hours, 8 hours or 24 hours at 37° C. A final concentration of 20 μM of CTZ was added and hGluc activity was measured immediately (exposure time=2 s). hGluc activity was detectable in 100% serum. ****P<0.0001. Error bar: mean±SD.

FIG. 42A illustrates images of mice, FIG. 42B-C illustrate images of stained tissue sections, and FIG. 42D graphically illustrates data, showing that mesenchymal stem cells home to tumor site and persist longer than in healthy mice. (FIG. 42A) 5 weeks after eGFP-231 were seeded i.v. into (NOD-SCID gamma) NSG mice, 1×10⁶ Fluc-RFP-MSC were administered systemically into both tumor-free (top) and tumor-bearing (bottom) mice. Then mice were injected i.p. with D-Luciferin (150 mg/kg in Dulbecco's phosphate-buffered saline (DPBS)) and in vivo Fluc activity was measured at different time points (2 hours, 6 hours, 24 hours, 7 days and 10 days after MSC infusion) using an IVIS Lumina to begin data acquisition 10 minutes after substrate administration (exposure time=60 s; n=4 in each group). MSC were cleared out faster in tumor-free mice. Color scale: Min=6.50e4; Max=7.50e5. Frozen sections of lungs of (FIG. 42B) tumor-free mice and (FIG. 42C) eGFP-231 tumor-bearing mice sacrificed 10 days after Fluc-RFP-MSC infusion were stained with anti-eGFP (green) and anti-Fluc (red) antibodies. MSC was observed to home to tumor niche. Scale bar: 50 μm. (FIG. 42D) Fluc activity measured at different time points was quantified and normalized to the time point of 2 hours. Error bar: mean±SEM. *P<0.05. n=4 in each group.

FIG. 43A illustrates images of stained tissue sections, and FIG. 43B graphically illustrates data showing that Gaussia luciferase (hGluc) is active in murine blood and the signal is elevated in tumor-bearing mice. (FIG. 43A) Frozen sections of lungs of tumor-bearing mice sacrificed 10 days after Dil-labeled hGluc-MSC administration were stained with DAPI and then imaged by fluorescence microscopy. MSC (red) were observed to home to tumor niche (dense blue). Scale bar: 100 μm. (FIG. 43B) 5 weeks after Fluc-RFP-231 were seeded i.v. into NSG mice, 1×10⁶ hGluc-MSC were administered systemically into both tumor-free and tumor-bearing mice. Then murine blood was harvested and hGluc activity was measured at different time points (6 hours, 24 hours, 7 days and 10 days after MSC infusion) with IVIS Lumina immediately after substrate was added. hGluc activity measured at different time points was quantified and normalized to the time point of 6 hours. Error bar: mean±SEM. *P<0.05. Exposure time=30 s. n=4 in each group.

FIG. 44 schematically illustrates an exemplary use of mechano-responsive cell system (“Scar Eraser”) to study, detect and treat Tissue Fibrosis. Fibrotic tissues, such as cirrhotic liver, can be treated with systemically infused engineered stem cells. The stem cells (gray) will sense the higher stiffness of the fibrotic foci within the diseased tissue (yellow) and selectively activate in those regions to secrete a therapeutic agent (green) to treat the fibrosis, such as MMPs.

FIG. 45 schematically illustrates an example of tracking cell fate with soluble reporters post-transplantation. In alternative embodiments, Cells are engineered with different lineage-specific secreted soluble reporters. In alternative embodiments, the engineered cells are infused into patient or animals. After the cell home to the specific niche and secrete reporters into the blood, a small portion of blood (or other biological fluids) is collected. The collected blood is then encapsulated with different fluorogenic substrates specific to each reporter into picoliter-sized droplets. With the presence of reporter, the droplet will become fluorescent, and the color of fluorescence reflects cell lineage. For example, stem cells are engineered with different exogenous soluble reporter enzymes after each lineage-specific promoter (e.g., bone promoter-Gluc, muscle promoter-HRP, etc.). The specific reporter enzymes can be expressed after the stem cell differentiated into the corresponding cell lineage (e.g., Gluc is expressed when the stem cell has differentiated into bone cell, etc.) and secreted out of the cells. After transplanting the engineered stem cells into the recipient, the differentiation and lineage ratio of the cells can be monitored by blood test for the secreted reporter enzyme in the blood. The blood test is coupled with ultrasensitive detection methods, such as integrated comprehensive digital droplet detection (IC 3D). The blood sample is compartmentalized into picoliter-size droplets in oil, containing one or no enzyme in each droplet, and the droplets containing reporter enzymes will react with their specific fluorogenic substrate. The fluorescent droplet can be detected with 3D particle counter.

FIG. 46 illustrates images showing an exemplary use of engineered stem cells as provided herein as a scientific tool “stiffness ruler” to measure tissue stiffness and study mechanobiology in vivo and in vitro. (Left panel) Promoters of listed genes responsive to specific ranges of stiffness are cloned from genomic DNA and subcloned into promoterless vectors to drive expression of florescent proteins. In alternative embodiments, the constructs are permanently transduced into mesenchymal stem cells (MSC) to produce stable engineered MSC cell lines. (Right panel). In alternative embodiments, reporters are only be turned on in the presence of the appropriate mechano-environment.

FIG. 47 schematically illustrates an example of early detecting of exemplary engineered cells as provided herein with soluble markers. HSC are engineered with exogenous soluble reporter enzymes (e.g. beta-galactosidase (beta-gal)) after specific promoter (e.g. beta-actin for constitutive expression). In alternative embodiments, after transplanting the engineered stem cells into patients, the reporter enzymes are expressed and secreted into blood, which can be detected with blood test. In alternative embodiments, the blood test is coupled with ultrasensitive detection methods, such as integrated comprehensive digital droplet detection (IC 3D).

FIG. 48A illustrates images of mice, and FIG. 48B graphically illustrates data showing that Luc-MSC homing to the metastatic niche in vivo. (FIG. 48A) shows the representative pictures of in vivo luciferase imaging of systemically infused Luc-MSC 12 hours after MSC infusion. (FIG. 48B) Quantification of luciferase activity of Luc-MSC in the lungs of eGFP-231 tumor-bearing and tumor-free nude mice at different time points following systemic infusion. MSC persisted longer in tumor-bearing mice than in tumor-free mice until they were cleared out in approximately 1 week. The in vivo luciferase imaging was performed with an IVIS Lumina at the indicated time points. Relative Luc Activity=Log₂ [(Luciferase read of the tested mouse infused with Luc-MSC)/(Luciferase read of control mice average injected with DPBS)]. i.e., the RLA of mice injected with DPBS=0. n=4 for tumor-bearing and n=3 for tumor-free nude mice. Error bar: mean±SEM. *P<0.05, **P<0.01.

FIG. 49A illustrates images of mice, and FIG. 48B graphically illustrates data showing that MRCS homing and specific activation in response to the metastatic niche in vivo. (FIG. 49A) Representative pictures of in vivo luciferase imaging of systemically infused MRCS-Luc 12 hours after infusion. (FIG. 49B) Systemically infused MRCS-Luc were turned on in the lungs of eGFP-231 tumor-bearing nude mice but not tumor-free mice. Relative Luc Activity (RLA)=Log₂ [(Luciferase read of the mouse infused with MRCS-Luc)/(Luciferase read of control mice average injected with DPBS)]. i.e., the RLA of mice injected with DPBS=0. RLA were measured and plotted for tumor-bearing and tumor-free mice at different time points following systemic infusion of MRCS-Luc. n=4 for tumor-bearing and n=3 for tumor-free nude mice. Error bar: mean±SEM. *P<0.05 and **P<0.01.

FIG. 49C and FIG. 49D illustrate images of Frozen sections of lungs of Luc-RFP-231 tumor-bearing NSG mice and tumor-free NSG mice, respectively, sacrificed 24 hours after MRCS-CD infusion were stained with anti-Luc (red) for lung metastasis, anti-CD (green) for cytosine deaminase expressed by MRCS-CD and DAPI (blue). MRCS-CD were observed to home to and specifically activated to express CD at tumor sites. White arrows indicate the co-localization of lung metastatic sites and MRCS-CD expressing CD (turned on). Scale bar=50 μm.

FIG. 50A and FIG. 50B illustrate images of Frozen sections of lungs showing that specific activation of MRCS-eGFP in response to the metastatic niche in vivo. MRCS-eGFP were observed to home to and specifically turned on at tumor sites in NSG mice. Frozen sections of lungs of (FIG. 50A) Luc-RFP-231 tumor-bearing mice and (FIG. 50B) tumor-free mice sacrificed 24 hours after MRCS-eGFP infusion were stained with anti-Luc (red) for lung metastasis, anti-eGFP for eGFP expressed by MRCS-eGFP (green) and DAPI (blue). White arrows indicate the co-localization of lung metastatic sites and MRCS-eGFP expressing eGFP (turned on). Scale bar=100 μm.

FIG. 51A-C illustrate images of Frozen sections of lungs showing that specific activation of MRCS in response to mechano-cues in the metastatic niche in vivo. (FIG. 51A-C) Frozen sections of lungs of Luc-RFP-231 tumor-bearing NSG mice and tumor-free NSG mice sacrificed 24 hours after eGFP co-transfected MRCS-CD infusion were stained with anti-Luc (red) for lung metastasis, anti-CD (magenta) for cytosine deaminase expressed by MRCS-CD and anti-eGFP (green) for MRCS-CD tracking. Second harmonic generation (SHG) imaging of collagen networks (cyan) was also presented and overlaid with IHC imaging. The data indicates that the MRCS-CD and its specific activation were co-localized with lung metastatic sites and collagen crosslinking and linearized networks. Scale bar=50 μm. Multiple high quality images were generated and processed with ImageJ and Matlab, and then tiled into a large, stitched image. Each representative picture was then extracted from the tiled image.

FIG. 52A-D graphically illustrate (FIG. 52A, FIG. 52D) and through images (FIG. 52B, FIG. 52C) show MSC with constitutive cytosine deaminase (CD) expression (CD-MSC) are able to kill cancer cells in the presence of 5-FC in vitro. The expression of cytosine deaminase (CD) was validated by (FIG. 52A) RT qPCR and (FIG. 52B) immunofluorescent staining. CD (green); DAPI (blue, nuclear counterstain). Native MSC (N-MSC) is included as a control in panel (FIG. 52A and FIG. 52C). In panel A, the CD mRNA expression of N-MSC was normalized to “1”. N-MSC does not express CD. (FIG. 52D) XTT assay was performed to show that CD expressing MSC are suicide agents in the presence of 5-FC at various concentrations. MSC proliferation is highly decreased only when both CD expressing MSC and prodrug 5-FC exist.

FIG. 52E-J illustrate images of stained breast cancer from a co-culture experiment conducted with CD-MSC and RFP expressing MDA-MB-231 breast cancer cells (231:MSC=2:1) with or without 800 μg/ml 5-FC. It shows that approximately 95% of breast cancer cells are killed and rest is apoptotic while no 5-FC control has a high confluency, showing CD-MSC-5-FC system is sufficient to kill adjacent breast cancer cells. RFP (red) and bright field (BF) are displayed. Scale bar=100 μm. Error bar: mean±SD. ***P<0.001.

FIG. 53 graphically illustrates data showing that engineered MSC can express highly increased levels of the enzyme matrix metalloproteinase-1 (MMP-1) to aid in degradation of excess collagen crosslinking formed during pathologic fibrosis when compared to native MSC (N-MSC). C-MSC were engineered to constitutively express MMP-1 using a CMV promoter. *P<0.05. Error bar: mean±SD.

FIG. 54A illustrates images of stained mice and FIG. 54B graphically illustrates data showing that the homing and retention of MSC to fibrotic and healthy livers after portal vein injection. MSC were engineered to express Firefly luciferase (Fluc). Relative Fluc activity was measured at each of the time points (6 hours, 12 hours, 24 hours, 48 hours, and 72 hours) using IVIS Lumina. Activity for each tested mouse image was normalized to control mouse images to negate background signals, then reported as a proportion as compared to the average diseased signal at 6 hours, which is 1 on this scale. Error bar: mean±SD.

FIG. 55A schematically illustrates a genetic circuit for a 2-state stiffness ruler, and FIG. 55B graphically illustrates data showing that a mechano-sensitive promoter (MSP) drives the expression of a Red Fluorescent Protein (RFP) reporting the ON-state coexpressed with an orthogonally targeted silencing construct that is cleaved by 2A peptide motif. The orthogonally targeted silencing construct comprises a Gal4 DNA binding domain (GAL4DBD) fused via a flexible linker domain to an epigenetic silencing domain (ESD). Examples of epigenetic silencing domains that can be used are KRAB or HDAC4. GAL4DBD-ESD binds to the Upstream Activation Sequence (UAS) and epigenetically silences the constitutive expression of a Green Fluorescent Protein (GFP) reporting the OFF state. This circuit is site-specifically integrated into a genetic safe harbor (GSH), such as AAVS1 in human cell lines and mROSA26 in murine cell lines. Site-specific integration is accomplished using Homology Directed Repair (HDR) and the CRISPR/Cas9 targeted endonucleases. (FIG. 55B) Example of genetic circuit output at varying stiffness. Fluorescent Protein (FP) output intensity varies at different input substrate stiffness. In this example at low stiffness RFP is not highly expressed as the mechano-sensitive promoter is not activated. Therefore, the GAL4DBD-ESD fragment is also not expressed and hence does not silence the otherwise constitutively expressed GFP. When cells are bound to substrates with progressively increasing stiffness, the GAL4DBD-ESD fragment progressively decreases GFP expression by silencing the mammalian constitutive promoter.

FIG. 56A schematically illustrates a genetic circuit for a multi-state stiffness ruler, and FIG. 55B graphically illustrates data showing that a series of mechano-sensitive promoters (MSP) individually drive the expression of multiple Fluorescent Proteins, (RFP, GFP, BFP). Each MSP has been tuned to be reversibly activated and deactivated over a specific stiffness range. Low stiffness for MSPa, medium stiffness for MSPb and high stiffness for MSPc. Each element of the genetic circuit is “insulated” by chromatin insulators (such as HS4 core insulator or others). These sequences block the upstream and downstream effects from adjacently expressed genes. Such sequencing also decreases long-term epigenetic silencing. Circuits elements are again inserted into a genetic safe harbor (GSH) site to least perturb the host cell/animal. (FIG. 56B) Example of Fluorescent Protein (FP) output intensity over a range of stiffness inputs. With cells on a low stiffness substrate only RFP is expressed strongly. At medium stiffness GFP is predominantly expressed. While at high stiffness BFP is mainly expressed.

FIG. 57A-C schematically illustrate use of an exemplary mechano-responsive CAR T cell to target and treat cancer metastases. The MRCS system can be adapted to T cell therapy. (FIG. 57A) Mechano-sensitive AND-gated Chimeric Antigen Receptor (CAR) T cells are formed by from peripheral blood mononuclear cells (PBCMs) isolated from patients by apheresis (1), then isolated T cells are engineered (2) using lentiviral constructs that carry a targeted CAR (such as HER2-CAR) that is only expressed when the cells bind to ECM with high stiffness. Lastly, these cells are expanded and infused into the patients (3). (FIG. 57B) Using the mechano-responsive promoter to drive the expression of CAR that targets typical solid tumor markers, such as HER2. These T cells only express the CAR when T cells are bound to stiff substrates. However, the T cells are still inactive until the HER2-CAR finds its target, to activate the T cell. (FIG. 57C) HER2 positive cells activate the T cell and lead to the killing of tumor cells.

Like reference symbols in the various drawings indicate like elements, unless otherwise stated.

### DETAILED DESCRIPTION

In alternative embodiments, provided are methods for detecting and treating disease states, including cancer, diabetes, fibrosis, and autoimmune diseases, by detecting increased mechanical modulus, or stiffness, or targeting tissues having increased mechanical modulus, or stiffness. Practicing these methods provides specific and localized detection assays and therapies for these disease states, including cancer, diabetes, fibrosis, and autoimmune diseases.

Provided are methods of cell monitoring and manipulating that enable modified cells to detect, respond to and manipulate niches of, for example, abnormal tissue characteristic of many disease states. This will allow for earlier and more accurate diagnosis as well as post treatment monitoring. Cells constantly interact with their niche which includes an array of complex biochemical and biophysical signals from, for example, the surrounding extracellular matrix (ECM). Although not appreciated historically, it has recently become evident that the physical and mechanical properties of cellular microenvironments (the so-called “mechano-niche”) regulate important cell functions.

Provided are methods for selectively delivering therapeutics to diseased regions. This will allow for more targeted and effective treatment with less harmful side effects. Provided are methods that take advantage of the endogenous ability of stem cells to respond to matrix stiffness to drive expression of reporters with stiffness-responsive promoters. In alternative embodiments, the promoters of genes upregulated in response to specific ranges of matrix stiffness capture and synthesize the regulatory inputs responsive to discrete ranges of stiffness. Using these promoters to drive expression of a reporter or therapeutic creates a mechano-responsive cell system (MRCS) that responds to ranges of matrix stiffness found in pathologic tissues.

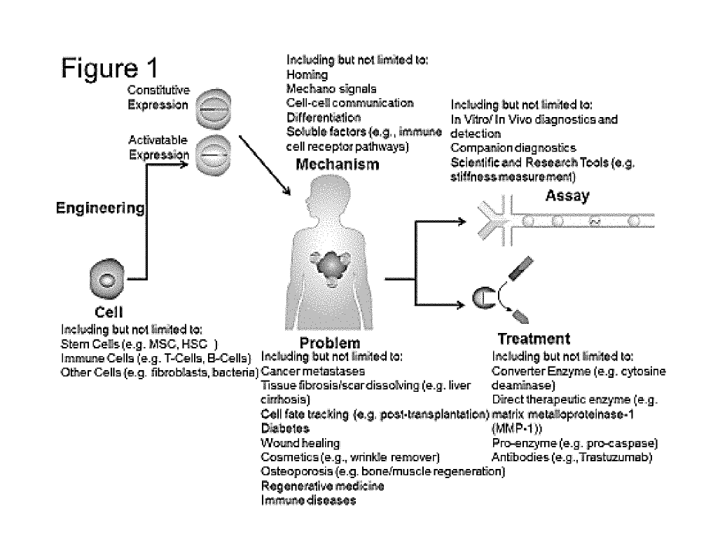

In alternative embodiments, provided are systems that employ engineered (e.g., genetically or recombinant, or non-genetically modified) cells that are able to target, detect abnormal cells or tissues of disease states. In addition, the transplanted cells are able to respond to cellular or niche characteristics including biochemical or physical markers to produce, e.g., reporter molecules for imaging and diagnostic purposes or therapeutics to treat a disease (FIG. 1).

In alternative embodiments, provided are platform technologies to track and monitor transplanted cells from minutes and hours, to days and to years in vivo relies on the measurement of secreted probes in biological fluids such as blood or urine that is coupled to a particular cellular function.

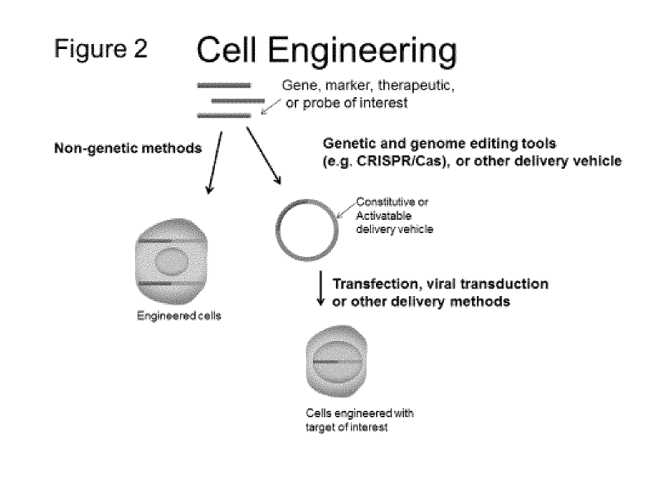

In alternative embodiments, provided are engineered or recombinant cells, or an engineering method, that changes the content of a cell to include direct therapeutic agents, converter enzyme, pro-enzyme, antibody, exogenous proteins, exogenous nanoparticles, or any molecule that originally does not exist in the cell (FIG. 2).

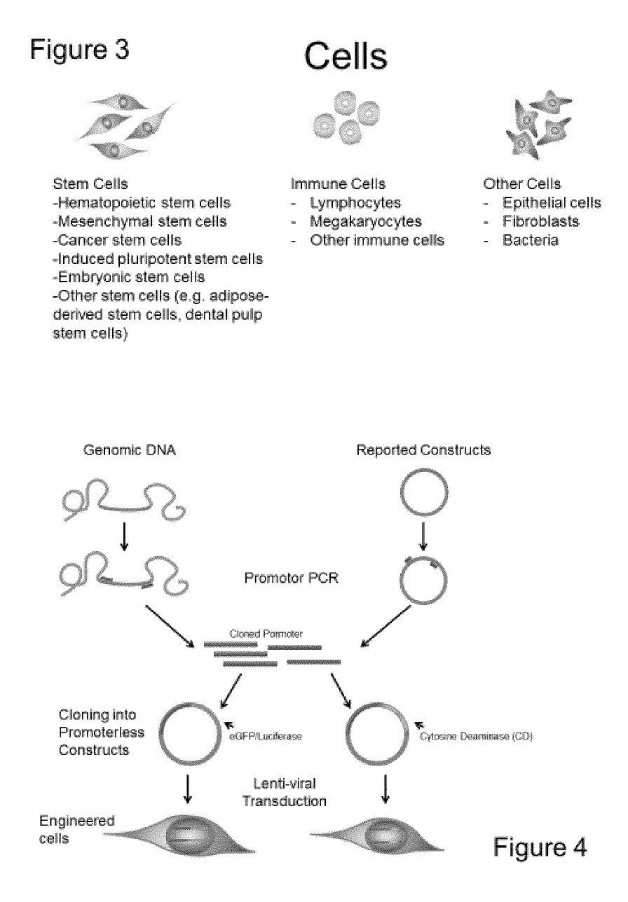

In alternative embodiments, the engineered or recombinant cell includes but not limited to stem cells (e.g., MSC, HSC, etc.), immune cells (e.g., lymphocytes, megakaryocytes, etc.), or any other cell (e.g., epithelial cell, fibroblasts, etc.) and microorganisms such as bacteria (FIG. 3). In alternative embodiments, the genetic engineering method is constitutive or activatable. In alternative embodiments, the gene for engineering is from genomic DNA or constructs (FIG. 4).

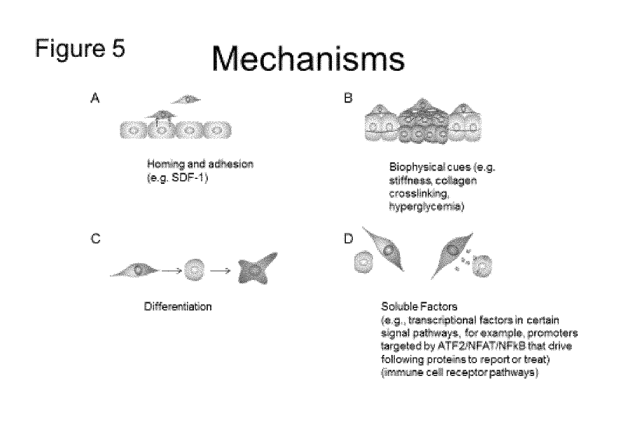

In alternative embodiments, the mechanism of the engineered cell responding or interfere with the system includes but not limited to differentiation, mechano-signals, cell-cell communication, soluble factors, extracellular environment, or in response to other factors (FIG. 5), where differentiation comprises of the engineered cell alters its location and cellular content upon changing the cellular type specificity from low to high, mechano-signals comprise of the engineered cell alters its location and cellular content upon receiving the stiffness and/or crosslinking signal from extracellular matrix or extracellular environment, cell-cell communication comprise of the engineered cell alters its location and cellular content upon interacting with other cells, soluble factors comprise of the engineered cell alters its location and cellular content upon receiving factors in the extracellular environment, extracellular environment comprise of the engineered cell alters its location and cellular content in response to the content in the extracellular environment, and other factors comprises of any chemical or condition that alters the location and cellular content of the engineered cells, including but not limited to proteins, nucleic acids, lipids, carbohydrates, small molecules, pH, temperature, radiation, or any other factors.

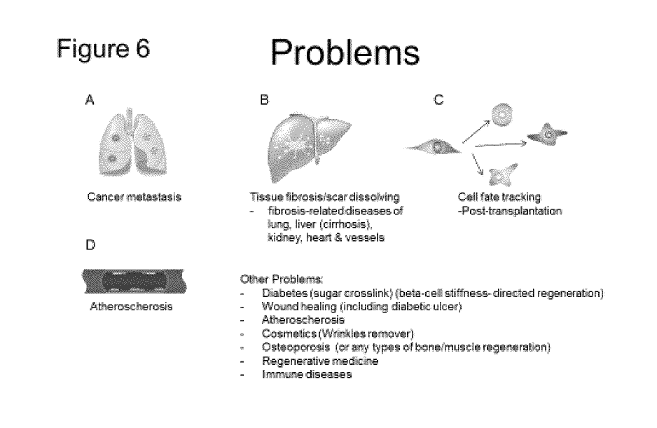

In alternative embodiments, provided are methods for treating or diagnosing diseases or conditions not limited to: cancer metastases, tissue fibrosis, cell fate tracking, diabetes, wound healing, cosmetics, osteoporosis, regenerative medicine, or immune diseases (FIG. 6).

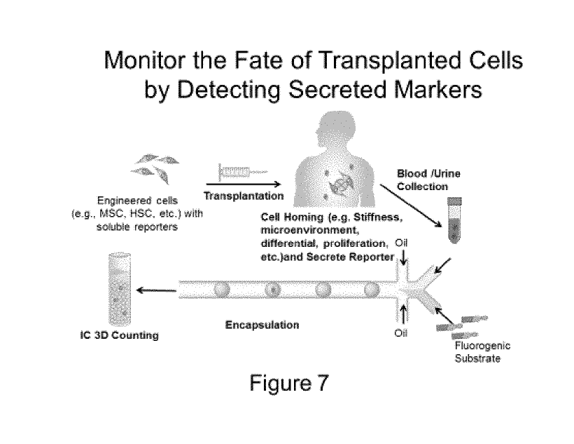

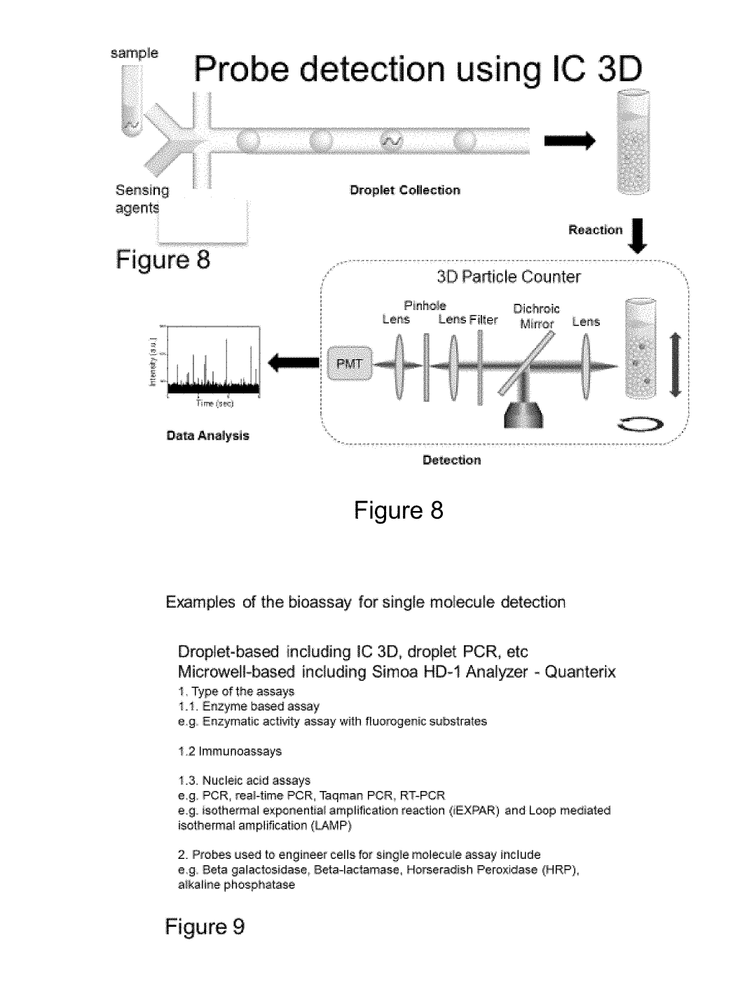

In alternative embodiments, provided are methods for detecting engineered cells with soluble markers (FIG. 7). Cells are engineered with exogenous soluble reporter enzymes expressed by nucleic acids under the control of specific stiffness sensing promoters (i.e., YAP/TAZ for stiffness sensing). After transplanting the engineered stem cells into patients, the reporter enzymes are be expressed after the cells home to specific niche (e.g., tumor niche) and secrete the enzymes into blood, which can be detected with blood test. The blood test is coupled with ultrasensitive detection methods, such as integrated comprehensive digital droplet detection (IC 3D) or other single molecule detecting technologies. In IC 3D, specifically, the blood sample is compartmentalized into picoliter-size droplets in oil, containing one or no enzyme in each droplet, and the droplets containing reporter enzymes can react with their specific fluorogenic substrate. The fluorescent droplet can be detected with 3D particle counter. In IC 3D, the reagents (e.g., blood sample containing targets, and sensors for targets) are mixed in oil, generating picoliter-size water-in-oil droplets that either contains one or no target. Within the droplet containing target, the sensor and targets can react and generate fluorescent product. The fluorescent droplet can be detected with 3D particle counter to determine the number of fluorescent droplet, which correspond to the number of target contained in the original sample (FIG. 8)

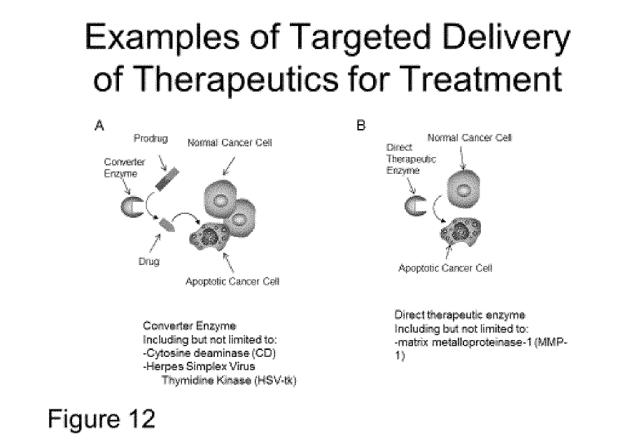

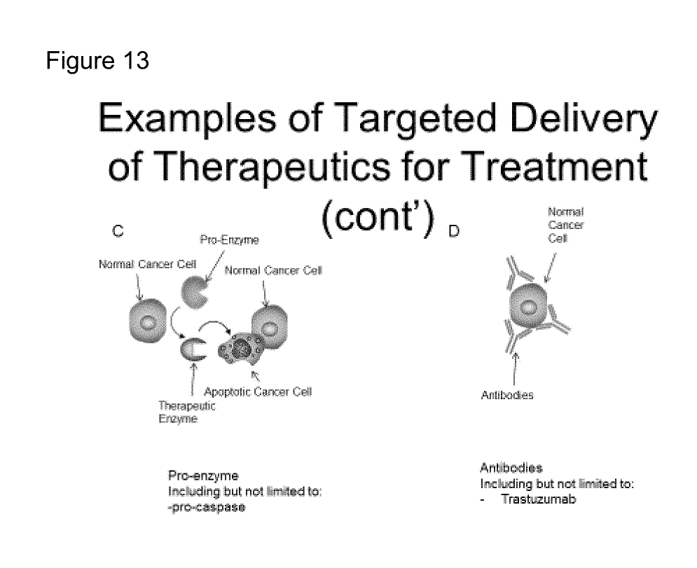

In alternative embodiments, provided are methods for delivering targeted therapies through the expression including but not limited to: converter enzyme, direct therapeutic enzyme, pro-enzyme, antibody, or any molecule that directly or indirectly aids in therapeutic processes (FIGS. 12 & 13). Converter enzyme (e.g., cytosine deaminase) comprises of protein or any other molecule that converts an inactive form of therapeutic agent into its active form. Direct therapeutic enzyme (e.g., MMP) comprises of enzyme that direct alters the content of other cell or extracellular environment. Pro-enzyme (e.g., Caspase-3) comprises of protein or any molecule produced by the engineered cell and alters it form from inactive to active in response to mechanisms described previously, and delivers therapeutic effects in its active form. Antibody (e.g., Trastuzumab) can comprise an immunoglobulin produced by the engineered cell and aids in therapeutic process directly or indirectly.

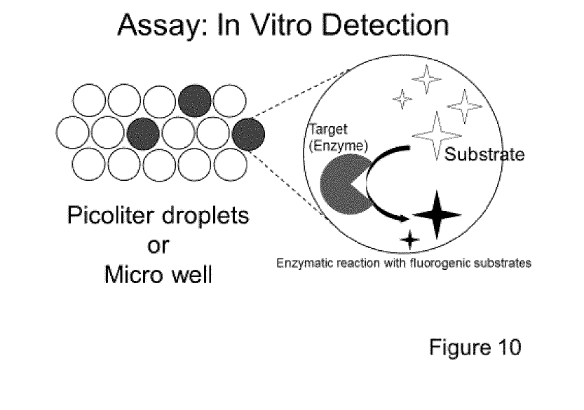

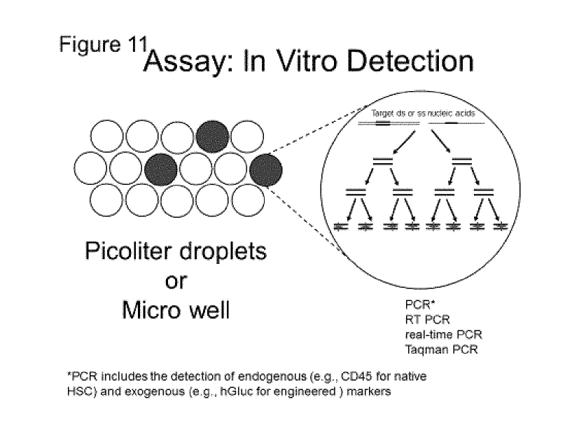

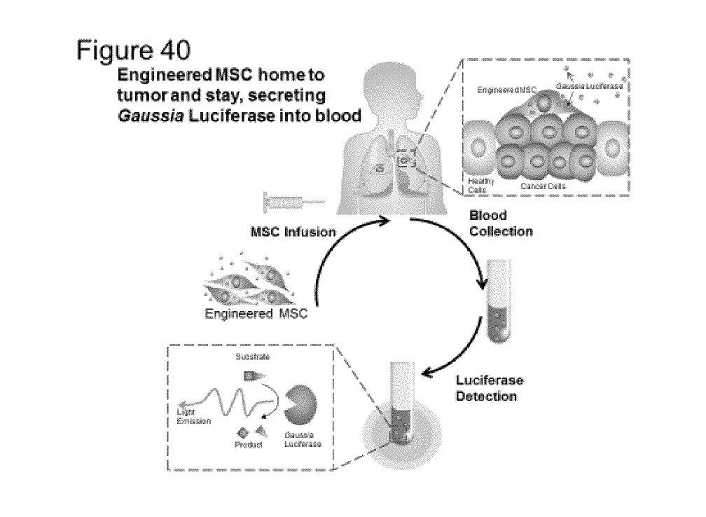

In alternative embodiments, the system enables assay for detection or diagnostics, companion diagnostics, or scientific and research tools. Assay for detection or diagnostics comprises of in vitro, in vivo, ex vivo, in situ or any other form of assay that enables the detection of the cellular location and/or content of the engineered cells (FIGS. 7, 14, 40 and 45). Companion diagnostics comprises of equipment and/or platform that enables the detection of cellular location and/or content of the engineered cells that current patented technology cannot achieve (FIGS. 8, 10 and 11). Scientific and/or research tools comprise of the usage of the engineered cell that facilitate the scientific study of biological processes (FIG. 46).

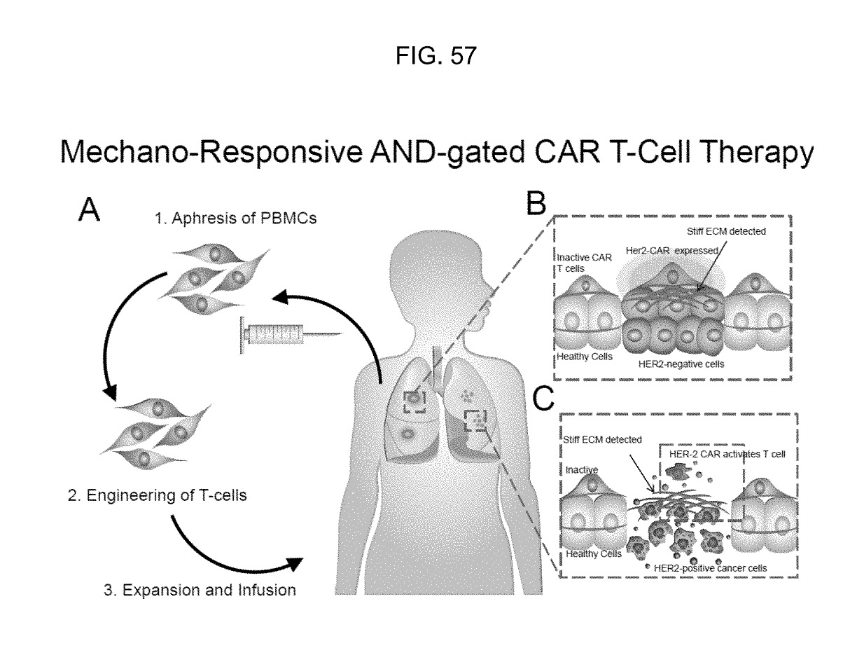

In alternative embodiments, provided are methods for making mechano-sensitive CAR T cells (or other cells including but not limited to, other immune cells or stem and adult cells or bacteria or other microorganisms) by using mechano-responsive promoter logic (i.e., logic-gates such as multi-input AND-gates or sequentially-stage AND-gates), and mechano-responsive promoter systems as provided herein. The biological and therapeutic activities of these cells are uniquely dependent on mechano-signals (including but not limited to LOXL1 and/or LOXL2 or biophysical stimuli such as hypoxia, and/or oxidative stress) and/or pathological markers (including but not limited to the tumor antigens such as HER2 or EGFRvIII) that initiate cell responses via, including but not limited to, engineered Chimeric Antigen Receptors (CARs) (FIG. 57). Targeting these biophysical cues can be used in combination with engineering cells to target other signals especially the biochemical cues. Together, embodiments described herein enable designing cells that can target biophysical and/or biochemical or other signals associated or surrounding cells to effectively treat a disease with minimized side effects.

In alternative embodiments, engineered cells as provided herein (including not limited to CAR T cells) are fused with or engineered to express novel single-chain variable fragments (scFv) or other synthetic promoters to target other aspects of biophysical cues such cross-linked biomarkers, hypoxic conditions, oxidative stress conditions in a logic dependent manner using logic-gated genetic circuits.

In alternative embodiments, provided are non-human transgenic animals, where varying strengths of mechano-signals are reported in the non-human transgenic animal (including but not limited to mouse, rat, rabbit, sheep or donkey). Varying strength of mechano-signals can be detected by an array of mechano-sensitive promoters and readout using an array of signaling moieties such as reporting molecules or devices including fluorescent proteins (including, but not limited to Blue Fluorescent Protein, Green Fluorescent Protein, Red Fluorescent Protein). In alternative embodiments, such genetic circuit elements are inserted using modular transfer vectors into genetic safe harbor locations.

Mesenchymal stem cells (MSC) can be used as vectors to generate MRCS. MSC are multipotent cells that can be derived from multiple adult tissues, including bone marrow and fat. In particular, MSC have been tested as therapeutic agents due to their intrinsic regenerative and immunomodulatory features. MSC are under investigation for treating a wide array of diseases including diabetes, myocardial infarction, stroke and autoimmune diseases^{[2]}. MSC are also the world's first manufactured stem cell product to receive clinical approval (i.e., PROCHYMAL® manufactured by Osiris was approved in Canada to treat graft-versus-host disease (GvHD))^{[2]} and for over 200 ongoing trials listed on clinicaltrials.gov, suggesting they may be a safe source for diagnostic and therapeutic uses in humans.

Engler et al. have previously performed microarray analysis of MSC exposed to different ranges of matrix stiffness to define genes specifically expressed under each set of conditions. We used this data as a starting point to design the MRCS to respond to matrix stiffness inputs. This includes cloning the approximately 3.0 kBp promoters of the TUBB3 (β3-tubulin, neurogenic lineage), MYOD1 (MyoD, myogenic lineage), and RUNX2 (RunX2 or CBFα1, osteogenic lineage) gene promoters from human genomic DNA with PCR. These promoters then were sub-cloned into a promoterless vector to drive expression of a destabilized version of red, yellow, and green fluorescent protein (TUBB3-RFPd, MYOD-YFPd, and RUNX2-GFPd) (FIG. 4, 46). GFPd has a half-life of 60-90 minutes, allowing near-real time imaging of promoter activity. We have successfully cloned these promoters from human genomic DNA. TUBB3 is induced at stiffness of less than one 1 kPa, MYOD1 is strongly expressed within the range of 9-25 kPa, and RUNX2 at stiffness greater than 25 kPa^{16, 17}. Moreover, the promoters bound to upstream transcriptional factors of RUNX2, YAP (Yes-associated protein) and TAZ (transcriptional coactivator with PDZ-binding motif, also known as WWTR1), were also sub-cloned to drive the expression of GFPd. YAP/TAZ has been reported as sensors and mediators of mechanical cues²⁰ via, for instance, cytoskeleton and Rho GTPase, regulating YAP/TAZ nuclear localization thus affecting YAP/TAZ function as transcriptional factors^{20, 21}. For all experiments, we utilized human bone marrow MSC from the Texas A&M Health Science Center (passage 3-6). For transduction of constructs, we used Lenti-viral transduction, which results in stable and robust engineered MSC cell lines. We have found culture of the constructed MRCS on tissue culture plastic does not lead to aberrant activation of promoters.

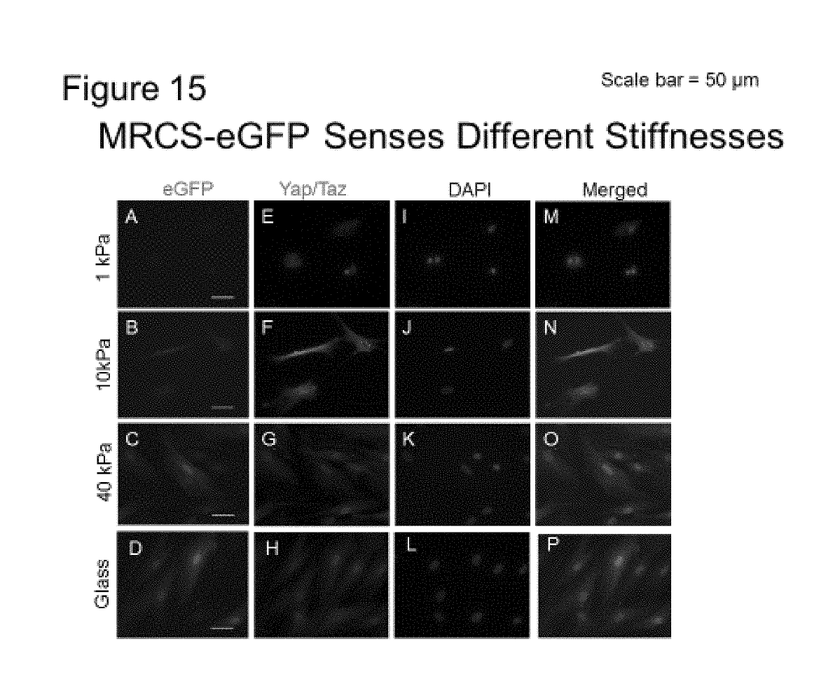

We chose TUBB3, MYOD1 and RUNX2 promoters for the initial screening process due to previous validated reports that variable levels of matrix stiffness are sufficient to induce their transcription^{[3, 4]}. We also screened multiple other gene promoters identified as regulated in response to matrix stiffness to ensure coverage of the entire range of physiological stiffness (including neurogenic: TUBB4, GDNF, and STAT3; myogenic: MYOG, PAX7, and MEOX2; osteogenic: BGLAP, SMAD1 and BMP6) as well as promoters bound to key upstream transcriptional factors of TUBB3, MYOD1 and RUNX2, YAP/TAZ (YAP (Yes-associated protein) and TAZ (transcriptional coactivator with PDZ-binding motif, also known as WWTR1, see e.g., DuPom et al (2011) Nature 474:179-183; Wrighton, (July 2011) Nature Reviews Molecular Cell Biology 12:404-405), for example). Notably, we have found that our MRCS with YAP/TAZ promoter is capable of sensing different stiffness (approximately 1 kPa, approximately 10 kPa and approximately 40 kPa) when seeded onto polyacrylamide hydrogels by expressing GFPd with proportional intensity (FIG. 15).

### EXAMPLES

### Example 1: Tumor Hunter: Mechano-Responsive Cell System to Study, Detect and Treat Cancer Metastases

Provided are mechano-responsive cell systems (“MRCS”) that can selectively detect and treat cancer metastases by targeting the unique biophysical and mechanical properties in the tumor microenvironment. Cancer metastases are responsible for over 90% of cancer deaths, however no current treatments directly target metastatic cancer. For breast cancer, in particular, about 1 in 8 American women will develop invasive breast cancer during their lifetime, leading to 40,000 deaths a year. Almost all breast cancer deaths are due to the spread of the cancer from the breast to other organs in a process called metastasis^{[5]} that is essentially incurable with a median survival of only 2 to 3 years. Resection of widespread metastases is often infeasible and chemotherapeutics, including taxanes and anti-metabolites, are discouragingly ineffective at treating disseminated disease and often associated with severe side effects. Current therapy for metastatic breast cancer therefore focuses on prolonging survival and palliation^{[5, 6]}.

An additional major challenge in treating cancer metastasis is that micrometastases (small numbers of cancer cells that have spread to distant organs) are often too small to be detected by traditional diagnostic tests such as computed tomography (CT) and magnetic resonance imaging (MRI). Indeed, only a small percentage of patients exhibit clinically detectable metastases at diagnosis. Importantly, micrometastases are known to be able to undergo a period of dormancy and escape chemotherapy. It is now thought that micrometastases, which may occur early during breast cancer progression, may account for cancer recurrence^{[6]}. Therefore, the ability to detect micrometastases will allow us to identify patients who are at high risk for relapse at early stages when treatment is most effective. Unfortunately, current micrometastases detection techniques are either not sensitive (i.e., CT and MRI) or require invasive biopsy procedures (e.g., sentinel lymph node, or lung biopsies), making them inappropriate for clinical application.

Therefore, there is clearly an enormous need for sensitive detection methods to identify metastases at early stages and for treatments specifically targeting breast cancer metastases to reduce mortality and side effects of current systemic therapies.

Cells constantly interact with their niche which includes an array of complex biochemical and biophysical signals from the surrounding extracellular matrix (ECM). Although not appreciated historically, it has recently become evident that the physical and mechanical properties of cellular microenvironments (the so-called “mechano-niche”) regulate important cell functions. Specifically, important roles for matrix stiffness (or elasticity) in driving breast cancer metastasis have been elucidated. Increased matrix stiffness, primarily driven by increased collagen deposition and crosslinking by lysyl oxidase (LOX) proteins, promotes breast cancer migration, invasion, cell plasticity, and eventual metastasis, primarily through regulation of integrin signaling^{[7, 8]}. Interestingly, LOX accumulation spatially correlates with the presence of metastases in both mouse models of metastasis and human patients^{[9]}. In mouse models of breast cancer metastasis, secretion of LOX by the primary breast tumor leads to collagen crosslinking in discrete areas of the lung that promote formation of metastases^{[9-13]}. Deposition of LOX at the metastatic niche correlates with both collagen linearization and formation of collagen-collagen covalent bonds in the lung parenchyma, both of which dramatically increase matrix stiffness [7]. We reason that the unique mechanical properties of the LOX-induced metastatic niche offer an intriguing target for the development of diagnostics and therapeutics specifically targeting lung metastases.

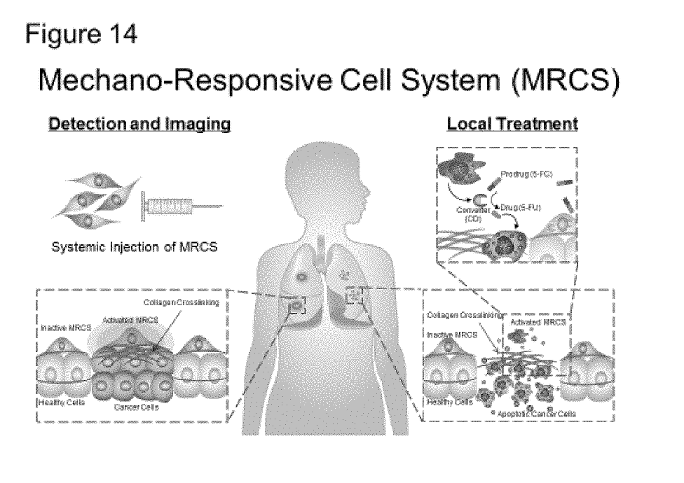

Provided are cell-based systems that responds specifically to mechano-environmental cues at the metastatic niche to target breast cancer metastases. Matrix stiffness is an appealing therapeutic target due to its intimate connection with formation of lung metastases and its long half-life (measured in years), making it refractory to development of resistance^{[14]}. Mesenchymal stem cells (MSC) are a promising vector for such an approach to generate a mechano-responsive cell system (MRCS) (FIG. 14). MSC are multipotent cells that can be derived from multiple adult tissues, including bone marrow and fat^{[15, 16]}. MSC are the basis for the first approved stem cell treatment in humans outside of bone marrow transplant (Prochymal, Osiris Therapeutics) and for over 200 ongoing trials listed on clinicaltrials.gov^{[17]} Importantly, systemically infused MSC preferentially home to and integrate with tumors in the body, including both primary breast tumors and lung metastases^{[18, 19]}. This is presumably due to recruitment of MSC by chemoattractants produced by the tumor, which makes them appealing vectors for localized delivery of therapeutics in cancer treatment^{[18, 19]}.

Provided are methods for manipulating tissue mechanical properties to regulate MSC fate: tissue and matrix stiffness is sufficient to drive expression of genes involved in MSC differentiation^{[3, 4, 20]}. Specifically, soft matrices, similar to the brain (Young's modulus of less than 1 kPa), direct MSC into a neurogenic lineage, whereas stiffer matrices (5 to 75 kPa), similar to muscle and bone, direct them into myogenic and osteogenic lineages through integrin and focal adhesion-dependent mechanisms. Importantly, the range of stiffness to which MSC respond encompasses those found in normal breast and lung tissues (less than 1 kPa), as well as invasive cancers and metastases (10-15 fold higher stiffness)^{[21].}

MSC differentiation is inherently a transcriptional program with each lineage defined by expression of characteristic transcription factors. This therefore allows us to use promoters regulating genes involved in MSC differentiation to drive expression of matrix stiffness-responsive reporters or therapeutics. Supporting our hypothesis that MSC can specifically respond to differences in tissue stiffness at the metastatic niche is the observation that MSC infused intravenously in a mouse model of cancer specifically assumed an osteogenic differentiation in the metastatic but not normal lung [18].

Provided is a MRCS to directly target the mechano-environmental cues of breast cancer metastases for localized and specific delivery of diagnostic reporters and anti-tumor agents.

### Methods

The endogenous ability of MSC to respond to matrix stiffness is used to drive expression of reporters with stiffness-responsive promoters. Promoters of genes upregulated in response to specific ranges of matrix stiffness capture and synthesize the regulatory inputs responsive to discrete ranges of stiffness. Using these promoters to drive expression of a reporter or therapeutic an MRCS is provided that responds to ranges of matrix stiffness found in the metastatic niche.

Secretion of LOX by the primary breast tumor leads to increased linearization and crosslinking of collagen at the metastatic niche associated with increased matrix stiffness^{[11]}. This is evident based on previous reports that 1) collagen linearization and crosslinking are robust surrogate markers of matrix stiffness, and 2) that exogenous MSC recruited to the metastatic lung assume an osteogenic differentiation profile associated with increased matrix stiffness not observed in the normal lung^{[18, 22]}. Here, we validated the MRCS in metastatic niche in vivo, and tested the feasibility of using MRCS to detect metastases and measure tissue stiffness in situ in vivo.

As a model of breast cancer metastasis to the lung, we utilized an MDA-MB-231 xenotransplantation model as MDA-MB-231 cells secreting large amounts of LOX, which leads to increased crosslinking of collagen fibrils in the lung that is essential for metastasis^{[9]}. In addition, inhibition of LOX is sufficient to prevent breast cancer metastasis of MDA-MB-231 cells^{[12]}. Briefly, MDA-MB-231 cells stably transduced with luciferase and RFP and suspended in Matrigel/PBS were orthotopically implanted/systemically infused into adult female nude mice. Seven weeks post-infusion, we monitored luciferase expression from metastasized cancer cells in the lung with an IVIS Imaging System. Upon noting significant increases in luciferase activity, compared to control group which was injected with only Matrigel or PBS, we then sacrificed the mouse and collected lung tissue, observing obviously growing RFP signal from metastasized cancer cells. The above-mentioned data shows that we have successfully established the in vivo MDA-MB-231 xenotransplantation model. Lungs will be further analyzed for the presence of metastases (cytokeratin (CK) staining) as well as evidence of LOX accumulation with immunostaining. An important control is mice injected with only Matrigel or PBS to understand normal patterns of LOX and CK staining. This experiment will establish important time points to understand the kinetics of metastasis for delivery of the MRCS.

Tumor-bearing mice were split into four experimental groups, with each group receiving one intravenous infusion of 1×10⁶ of one of the MRSC reporters (TUBB3, MYOD1, RUNX2 and YAP/TAZ/GFPd); control, tumor-free mice will similarly be grouped and infused. Infusion of 1×10⁶ MSC is sufficient to efficiently deliver the MRCS to the metastatic niche; previously studies indicate that one week post-infusion, MSC that are passively entrapped in the lung microvasculature have been cleared, with the remaining MSC specifically located at metastases^{[22]}.

MSC can be used as passive sensors and vectors of the mechano-environment. We investigated MRCS activation at multiple time points to determine the earliest time point at which the MRCS are specifically and robustly activated to minimize any potential biological effects (e.g., secretion of proteases and ECM components^{[19]}) that MSC exert to modify their local environment in metastases. Mice were sacrificed 1, 2, 3, and 7 days following infusion to investigate the clearance of the MRCS from the lung and activation status of our reporters assayed by confocal microscopy as described below. We additionally stained for makers of metastasis and the metastatic niche as described above to determine if areas of reporter activation correlate with metastases and LOX. We also determined the correlation between reporter intensity and stiffness of the surrounding matrix (using atomic force microscopy (AFM) microindentation). Promoter engineering allowed us to achieve sufficiently robust correlations between MRCS signal with tissue stiffness to generate a cell-based stiffness “ruler” as an entirely novel method of interrogating the mechanical properties of biological tissues in situ in vivo.

Provided are methods to selectively deliver imaging and therapeutic agents to the metastatic lung mechano-environment. To determine which reporter construct (TUBB3, MYOD1 RUNX2 or YAP/TAZ) is most suited to this task, we perform image-based analysis of lung sections. Briefly, Provided are methods: 1) quantify the integrated fluorescent intensity of each of the GFPd reporters in the lung, 2) bin the intensities into “no”, “low” and “high” groups, and 3) quantify average distance between metastases and/or LOX accumulation and reporter intensity in the “high” bin. The reporter with the shortest distance between “high” reporter activity and metastases/LOX accumulation will be selected as the metastatic niche specific promoter for targeted delivery in subsequent experiments. Provided are analysis on lung tissue extracted at the time points described previously to determine the optimal time following MRCS infusion that produces robust and specific signal at the metastatic niche.

Provided are methods for detecting micrometastases or changes in the lung parenchyma characteristic of metastasis in high-risk patients. As a proof-of-principle, we engineered our MRCS to drive expression of destabilized luciferase in lieu of GFPd (MRCS-Luc). Following in vitro characterization of luciferase activity in response to discrete ranges of stiffness as described for fluorescence reporters, we administered the MRCS-Luc cells to mice at multiple time points following xenotransplantation of MDA-MB-231 cells not engineered to express luciferase. We measured luciferase activity in the lung in vivo before sacrificing the mice and collecting the lungs. The lungs were analyzed for metastatic burden and LOX immunostaining as described above to determine if these markers of metastasis and pre-metastatic niche formation correlate with luciferase signal from MRCS-Luc.

To locally treat breast cancer metastasis to the lung, we utilized the reporter most specifically and robustly activated at the metastatic niche to locally activate a pro-drug. As intravenous delivery of MSC, used in most clinical trials, leads to initial entrapment of large numbers of MSC in the pulmonary vasculature, localized activation of a pro-drug, rather than constitutively expressing a drug, at only the metastatic niche is desirable to avoid potential adverse toxicity in the pulmonary and other organ systems^{[18, 22]}. To these ends, we engineered the MRCS to express a pro-drug convertase in response to specific ranges of matrix stiffness found at metastases. In response to appropriate stiffness, the convertase will convert a systemically administered, inactive pro-drug into an active drug capable of killing both the MSC and nearby cancer cells via the bystander effect^{[23]}. This approach will not only more effectively target breast cancer metastases, but also avoid the side-effects of systemic therapies.

To design a MRCS for local drug activation, we utilized the reporters that were established as specific to the metastatic niche above. To the identified reporter constructs we replaced the GFPd with the gene for cytosine deaminase (CD). CD acts as a pro-drug convertase, converting the pro-drug 5-fluorocytosine (5-FC) into the potent anti-metabolite 5-fluorouracil (5-FU). This leads to localized activation of 5-FC via the bystander effect in which the apoptotic MRCS locally releases CD^{[23]}. This technique has shown great promise and is currently the basis of a clinical trial using neural stem cells (NSC) for the treatment of glioblastoma^{[23, 24]}. In addition, we made use of a vector in which CD is constitutively expressed as an important control to understand and quantify pulmonary and systemic toxicity of global MSC activation of 5-FC. MSC will be transduced as described above.

For in vivo animal experiments, nude mice were infused intravenously (i.v.) via the tail vein with FLuc-RFP-231 cancer cells. Six weeks after cancer seeding, the mice were injected intraperitoneally (i.p.) with D-Luciferin (150 mg/kg in DPBS) to observe the cancer signal from the Firefly luciferase (Fluc). The animals were divided into four treatment groups: C-MSC (constitutively expressed CD cells), MRCS-CD, N-MSC (native MSC control) and PBS control. MSC or PBS was then infused i.v. to both tumor-bearing and tumor-free healthy control mice at Day 0. The mice were treated with i.p. injections of cancer prodrug (5-FC, 00 mg/kg in DPBS) twice per day at 12 hour intervals for 5 days (Day 1-5), then once per 24 hours for 2 more days (Day 6-7). Fluc activity was then observed after treatment in vivo on Day 9 using IVIS Lumina imaging system. Image acquisition began 10 minutes after D-Luciferin administration. One mouse from each experimental group was euthanized on Day 1 and Day 9 for ex vivo tissue imaging and assays. We validated regulation of CD by our MRCS in response to matrix stiffness with the hydrogels described previously. We first correlated CD transcription/translation with local stiffness by using atomic force microscopy (AFM) followed by staining for CD (Abcam). To verify local production of functional CD and the efficacy of the bystander effect, we co-cultured MDA-MB-231 cells with our MRCS on hydrogels with different stiffness, with 5-FC added to the culture. We measured local stiffness with AFM and assay for apoptosis (TUNEL) to determine if apoptosis correlates with stiffness.

To examine the efficacy of our MRCS at treating lung metastases, we made use of the MDA-MB-231 xenograft model described above to explore the effects of our localized therapy on disseminated breast cancer. After establishment of lung metastases, as measured with luciferase imaging, we infused 1×10⁶ of our MRCS-CD. After allowing sufficient time for MRCS-CD activation (empirically determine in previous in vivo and in vitro experiments), we administered a single systemic infusion of 5-FC in normal saline via intraperitoneal injection; two days later mice were sacrificed and frozen and paraffin embedded lung sections analyzed by immunohistochemistry and immunofluorescence. Important controls include tumor-free mice and MDA-MB-231 xenotransplanted mice infused with un-transduced MSC and with MSC constitutively expressing CD.

### Results

Primary measures of outcome include 1) number of lung metastases (stain with anti-luciferase or CK antibody), 2) overall metastatic lung burden (from luciferase imaging of the living mouse and real time PCR of genomic DNA from lung tissue for luciferase gene normalized to mouse-specific GAPDH), and 3) apoptosis in endogenous lung tissue (TUNEL staining in lungs). All measures were quantified and analyzed for significant differences between experimental and control conditions. These experiments allowed us to determine if our therapy is efficient at eliminating metastases and more selective, sparing normal lung tissue from the deleterious effects of chemotherapy.

The MRCS with engineered reporter-eGFP construct (MRCS-eGFP) showed the ability to sense different stiffness and selectively activate GFP expression only on stiff substrates, as seen by immunofluorescence imaging in vitro (FIG. 15). MSRC-eGFP plated on soft, neurogenic substrate (1 kPa) failed to show GFP expression (FIG. 15A), but GFP could be seen on stiffer, osteogenic substrates (greater than 40 kPa) (FIGS. 15C and 15D). YAP/TAZ localization was also observed to be regulated by substrate stiffness. YAP/TAZ is deactivated and localized in the cell cytoplasm on softer substrates (neurogenic, 1 kPa and myogenic, 10 kPa) (FIGS. 15E and 15F) but activated and localized in the cell nuclei on stiffer substrates (FIGS. 15G and 15H). Nuclear colocalization can be seen from DAPI nuclear counterstain.

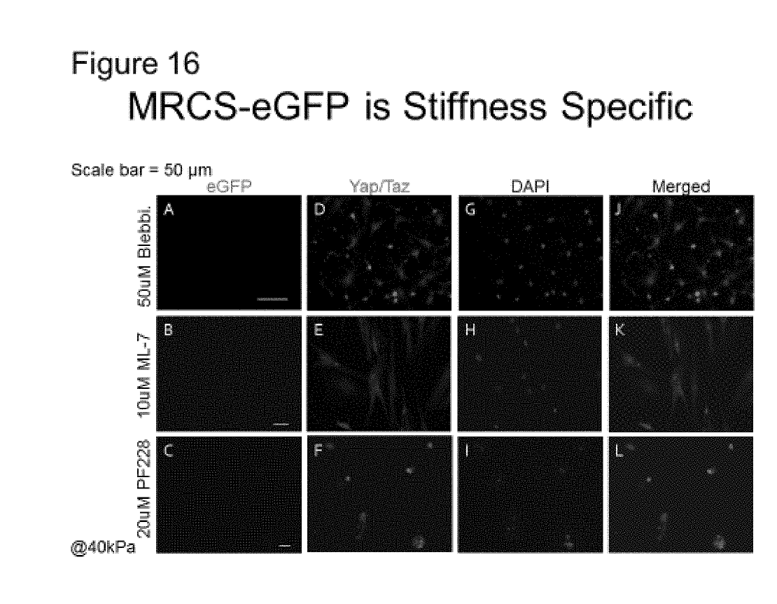

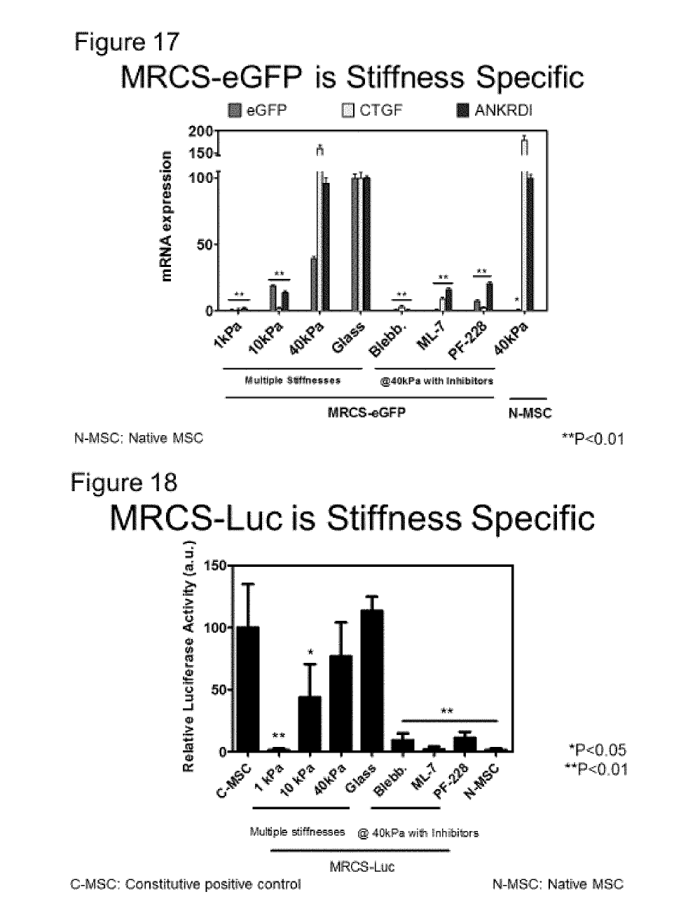

MRCS-eGFP sensing was also shown to be reversibly stiffness-dependent (FIG. 16). Cells plated on stiff, activating substrate (approximately 40 kPa) were deactivated with the addition of blebbistatin and ML-7 (myosin light-chain kinase inhibitors) or PF228 (a focal adhesion kinase inhibitor). This was seen from the lack of GFP fluorescence (FIG. 16A-C) and from the localization of YAP/TAZ to the cytoplasm (FIG. 16D-F). Real-time RT-PCR was performed on the seeded MRCS-eGFP cells to quantify relative mRNA expression levels of eGFP and two downstream factors of YAP/TAZ, CTGF (Connective Tissue Growth Factor) and ANKRDI (ANKyrin Repeat Domain-containing protein 1) (FIG. 17). Expression levels were significantly different between cells plated on soft substrates (1 kPa and 10 kPa) and cells plated on stiff substrates (40 kPa and glass). Expression levels were also found to be significantly lowered in the presence of the aforementioned inhibitors when compared to uninhibited cells on stiff, activating substrates. N-MSC plated on stiff substrate was a control. This data confirms the specific stiffness dependence of MRCS-eGFP gene expression.

The MRCS with engineered reporter-Luc construct (MRCS-Luc) was also found to be stiffness specific in vitro, similar to MRCS-eGFP (FIG. 18). Relative luciferase activity levels were significantly lower for MRCS-Luc plated on soft substrates (1 kPa and 10 kPa) and cells plated on stiff substrates (40 kPa and glass). Expression levels were also found to be significantly lowered in the presence of blebbistatin, ML-7, or PF228 when compared to uninhibited cells on stiff, activating substrates. C-MSC constitutively expressing luciferase served as a positive control, and N-MSC with no luciferase activity served as a negative control.

The MRCS with engineered reporter-CD construct (MRCS-CD) also senses different substrate stiffness in vitro (FIG. 19). MSRC-CD plated on soft substrates (1 kPa) failed to show GFP expression (FIG. 19A), but CD could be seen on stiffer, osteogenic substrates (>40 kPa) (FIGS. 19C and 19D). YAP/TAZ localization was also observed to be regulated by substrate stiffness (FIG. 19E-H).

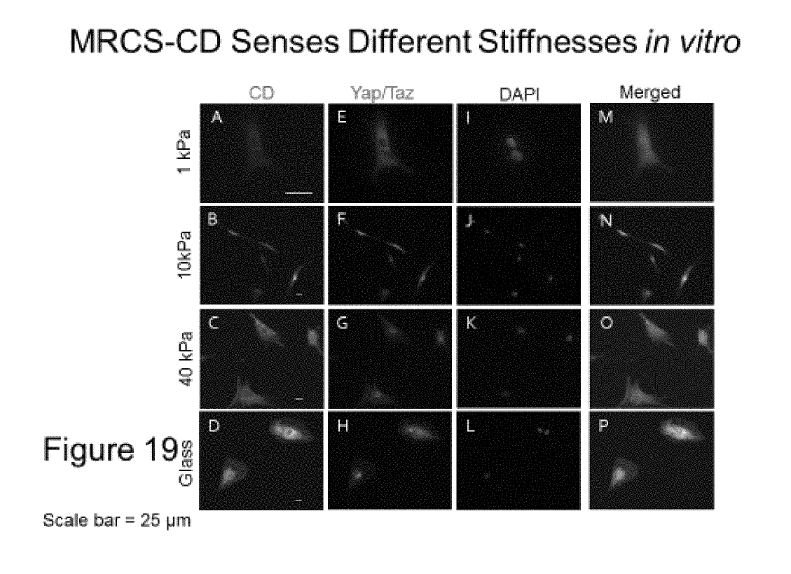

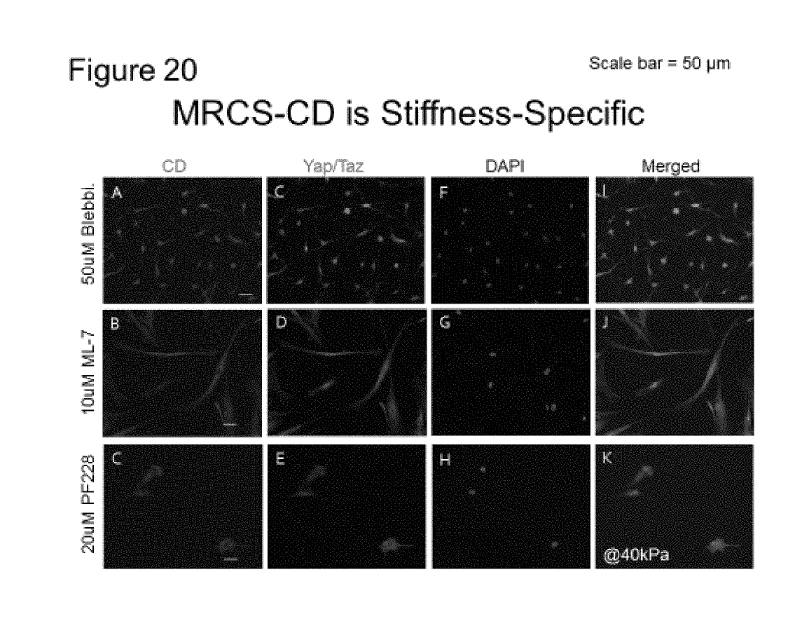

MRCS-CD sensing was also shown to be reversibly stiffness-dependent (FIG. 20). Cells plated on stiff, activating substrate (approximately 40 kPa) were deactivated with the addition of blebbistatin and ML-7 (myosin light-chain kinase inhibitors) or PF228 (a focal adhesion kinase inhibitor). This was seen from the lack of GFP fluorescence (FIG. 20A-C) and from the localization of YAP/TAZ to the cytoplasm (FIG. 20D-F).

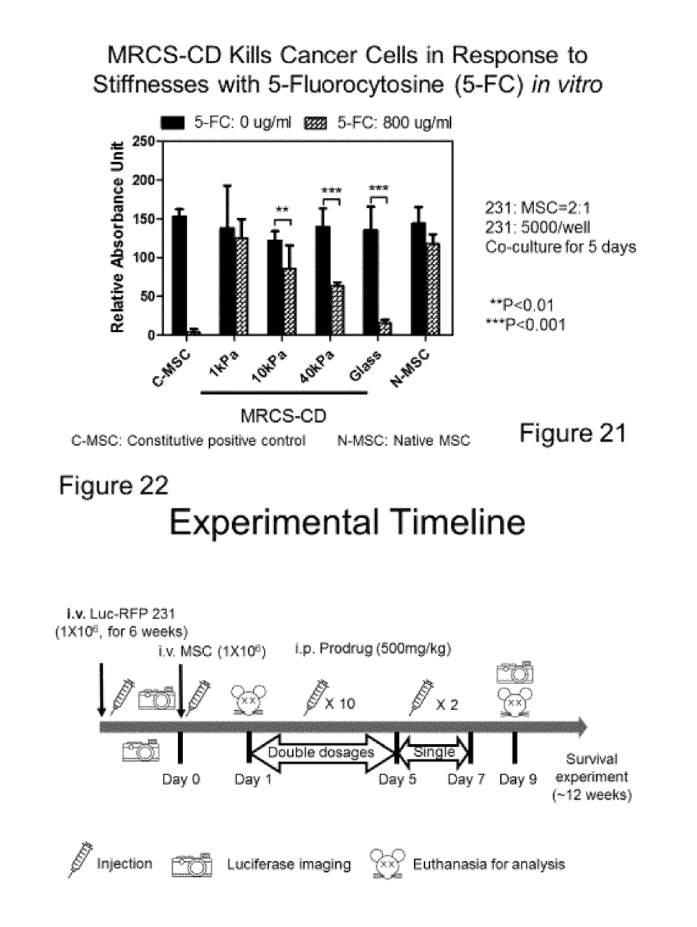

To test the functional effect of MRCS-CD at treating cancer, MRCS-CD were co-cultured with luciferase-expressing MDA-MB-231 human breast cancer cells (2:1 ratio of 231 cells to MRCS) (FIG. 21). Cells were co-cultured on substrates of different stiffness, varying from soft (1 kPa) to glass, for 5 days. Cells were co-cultured in media containing 5-FC (800 μg/mL) or without 5-FC. XTT assay was then used to determine the relative amount of cell proliferation under each condition. All data was normalized to a control sample with only MDA-MB-231 cells and no MRCS. Results show significantly decreased cancer cell proliferation in the presence of 5-FC when the MRCS-CD were co-cultured with 231 cells on stiffer (10 kPa and higher) substrates. The change in proliferation was not significant between 5-FC and no 5-FC on soft (1 kPa) substrate. C-MSC control which constitutively expressed CD saw the most drastic decrease in cancer cell proliferation, whereas N-MSC control with no expressed CD saw no significant change in cancer cell proliferation. From this experiment we can conclude that increased substrate stiffness which caused increased CD expression led to decreased cancer cell proliferation in the presence of cancer prodrug 5-FC.

From the in vitro experiments, we have concluded that MRCS can be engineered to respond specifically to different substrate stiffness and selectively express genes of interest. The cells could then be used to express reporter genes such as eGFP or luciferase for detection purposes, or therapeutic genes to aid in targeted treatment.

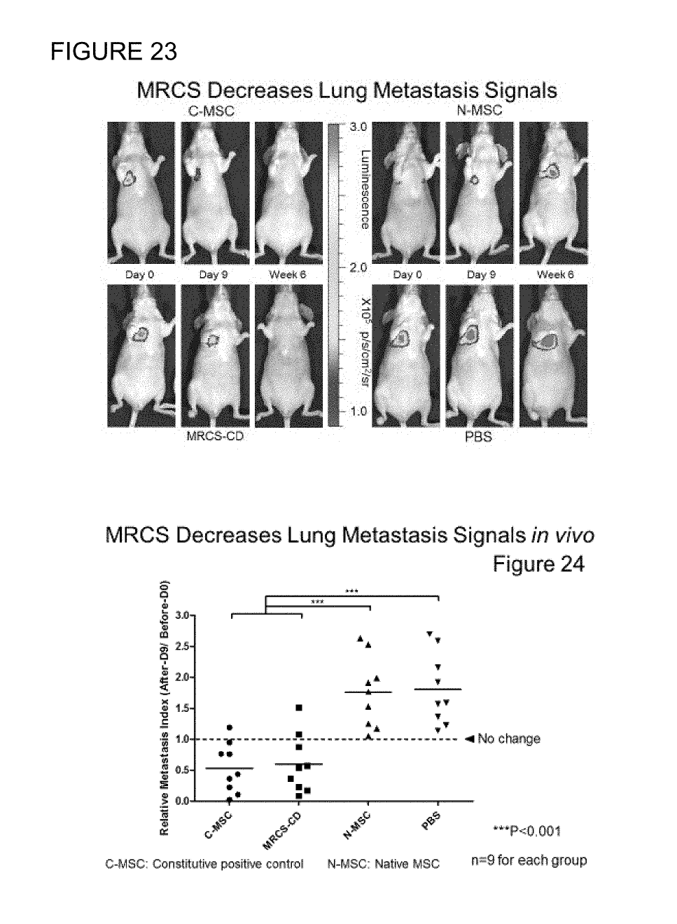

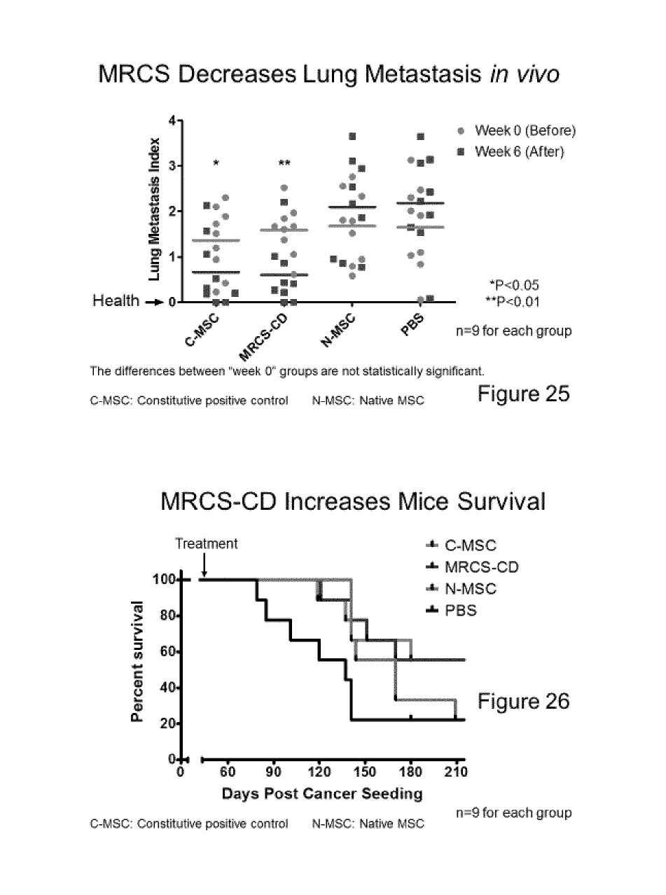

In vivo experiments proceeded as described above and as seen in FIG. 22. Two repeats of the experiment are shown here imaged for luciferase cancer signal at Day 0, Day 9 and Week 6 after treatment (FIG. 23). The signals were quantified, and the signal at Day 9 after treatment was divided by the signal at Day 0 before treatment to obtain a fold change (relative metastatic index, RMI) (FIG. 24). A RMI of 1 indicates no change before and after treatment. Data show C-MSC and MRCS-CD treatment groups had significantly reduced RMI as compared to N-MSC and PBS treatment groups. Long term quantification shows the same trend, when signals at Week 6 after treatment were normalized to a tumor-free control mouse (FIG. 25). Data show C-MSC and MRCS-CD treatment groups had significantly reduced lung metastatic index as compared to N-MSC and PBS treatment groups.

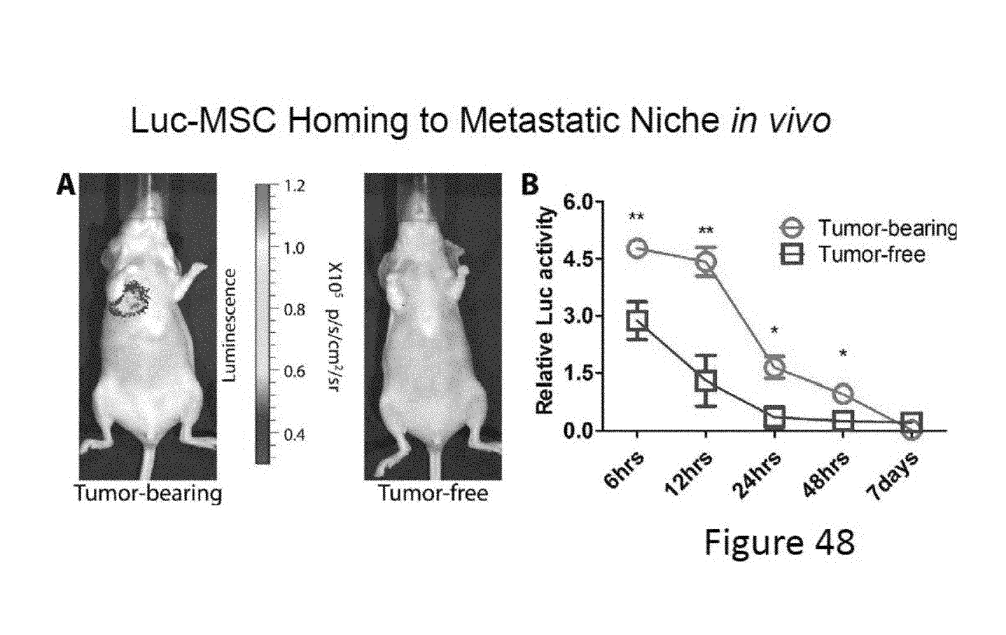

In order to examine whether MSC engineered to constitutively express firefly luciferase (Luc-MSC) are able to home to metastatic sites in the lungs, we systemically infused Luc-MSC to mice hosting human eGFP-231 breast cancer cells in the lung and tumor-free controls. We found that Luc-MSC homed to and persisted in lung metastatic sites (FIG. 48).

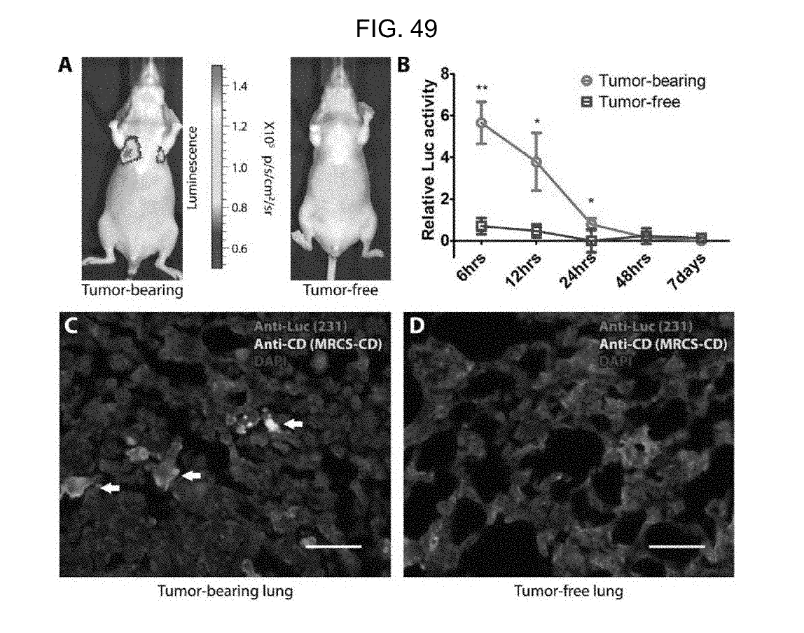

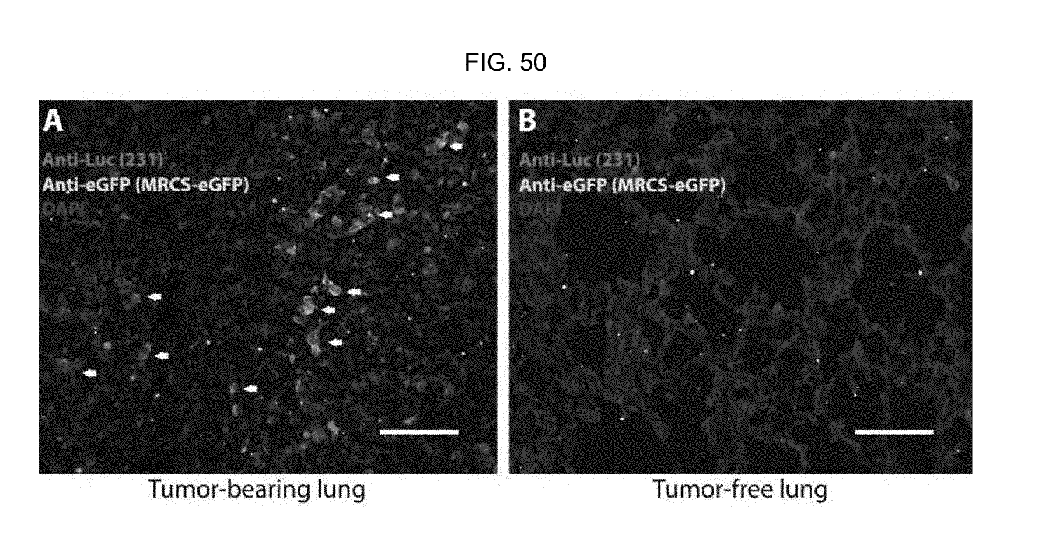

Next, we investigated whether MRCS can home to and be specifically activated at the tumor sites using MRCS-Luc which serves as a surrogate for MRCS-CD and allows us to readily track transplanted MRCS and monitor their activation using induced luciferase in vivo. We demonstrated that systemically infused MRCS-Luc homed to and were induced to express luciferase only in the tumor sites in the lung of eGFP-231 tumor-bearing mice (FIGS. 49A and 49B). The observed luciferase signal, which reflects the collective functional outcome of MRCS homing and activation at tumor sites, persisted in tumor-bearing mice for up to 1-2 days (FIG. 49A). We also confirmed the in vivo homing and activation of MRCS-CD in Luc-RFP-231 tumor-bearing mice using ex vivo immunohistochemistry (IHC). We demonstrated MRCS-CD were co-localized with and locally activated to express CD at cancer sites in lung sections of tumor-bearing (but not tumor-free) mice (FIGS. 49C and 49D). Similar results were observed with the infusion of MRCS-eGFP (FIG. 50). Collectively, these data demonstrate that MRCS selectively home to and are specifically activated at the metastatic niche in vivo.

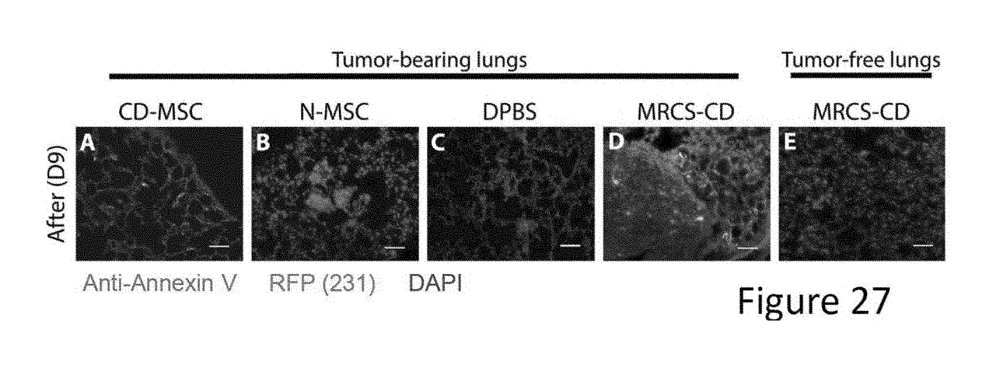

Staining for Annexin V to measure apoptosis showed the specific activation of MRCS-CD at metastatic sites (FIG. 27D), whereas no comparable Annexin V signal could be seen on tumor-free tissue (FIG. 27E). CD-MSC treated group stained positive for Annexin V non-specifically indicating extensive tissue damage (FIG. 27A). Mice treated with N-MSC or DPBS stained positive for tumor but not for Annexin V (FIGS. 27 B and 27C), indicating either native MSC or DPBS infusion does not cause cytotoxicity.

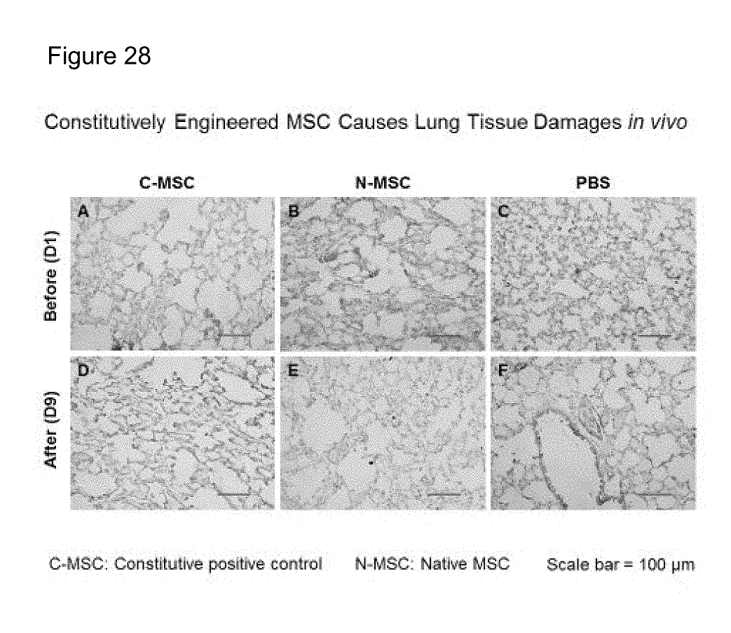

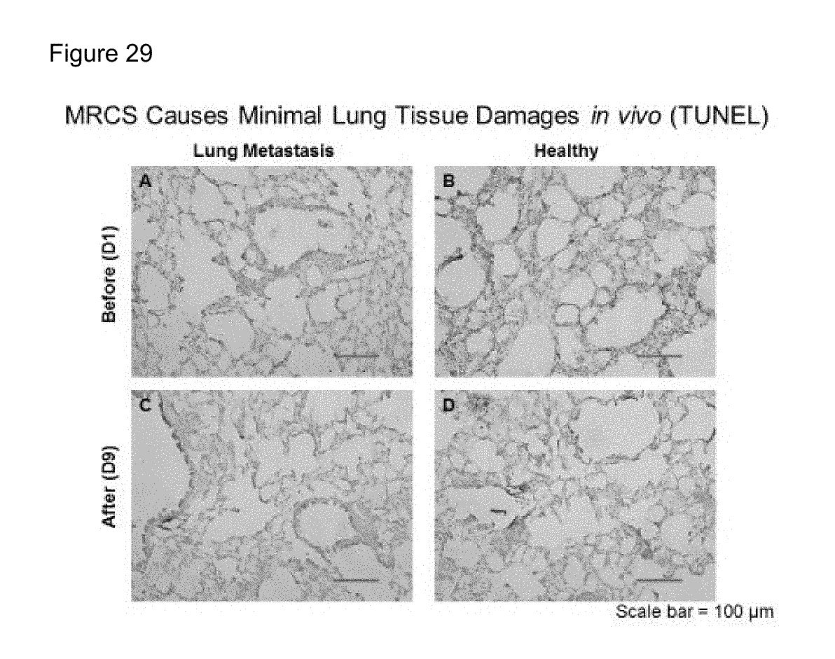

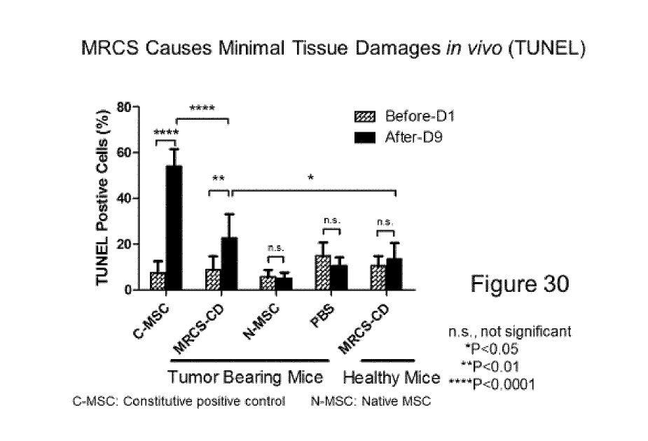

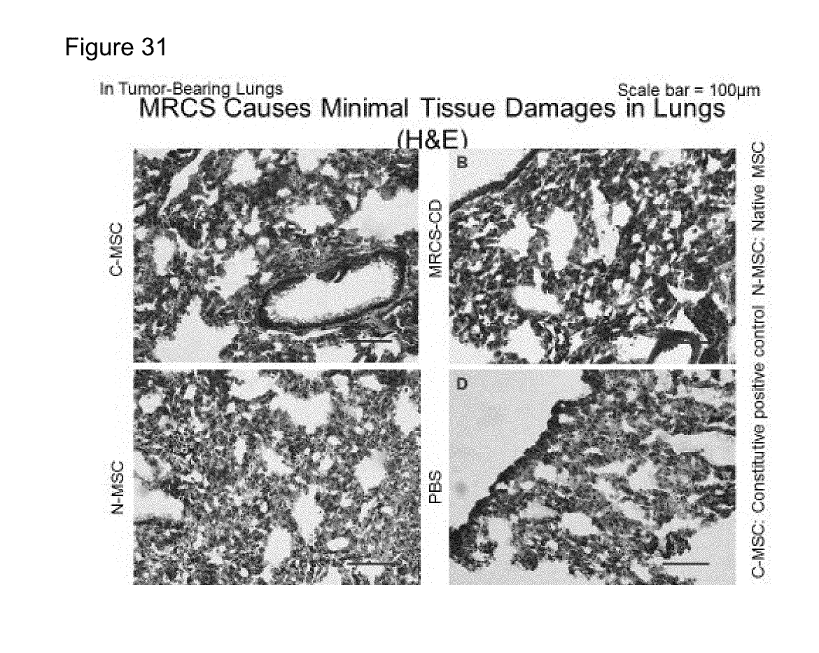

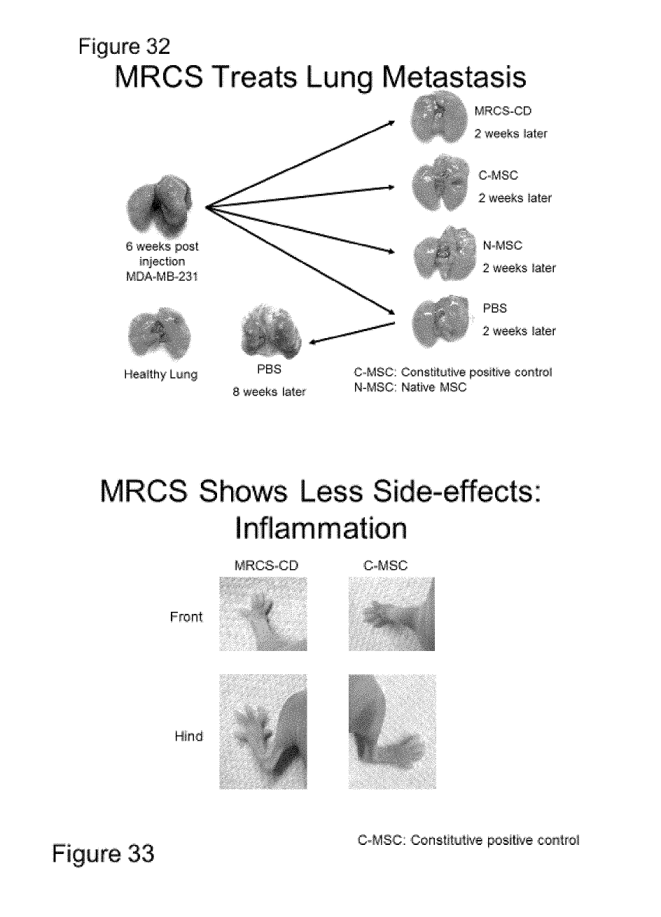

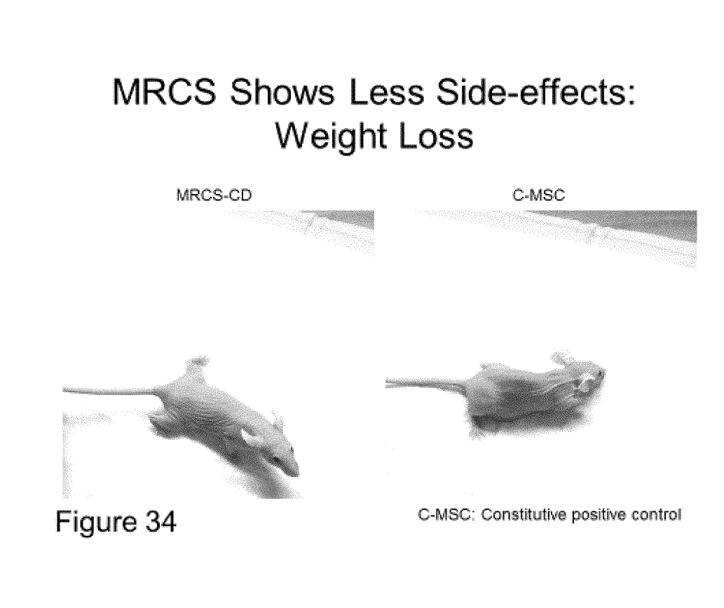

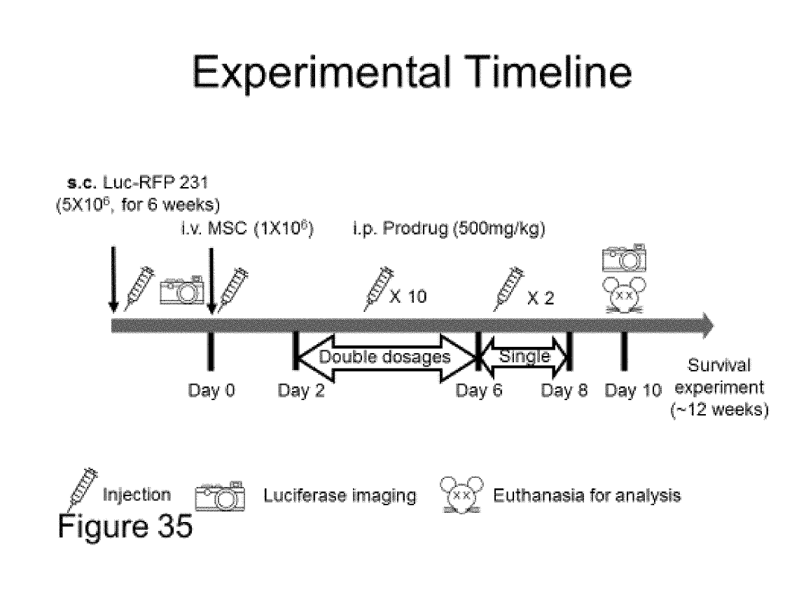

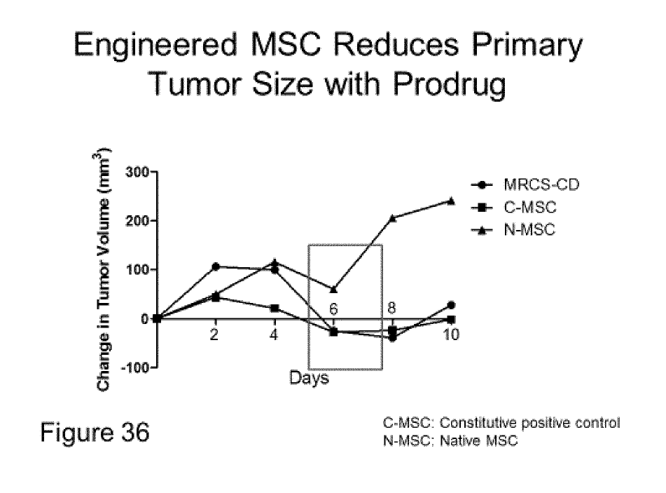

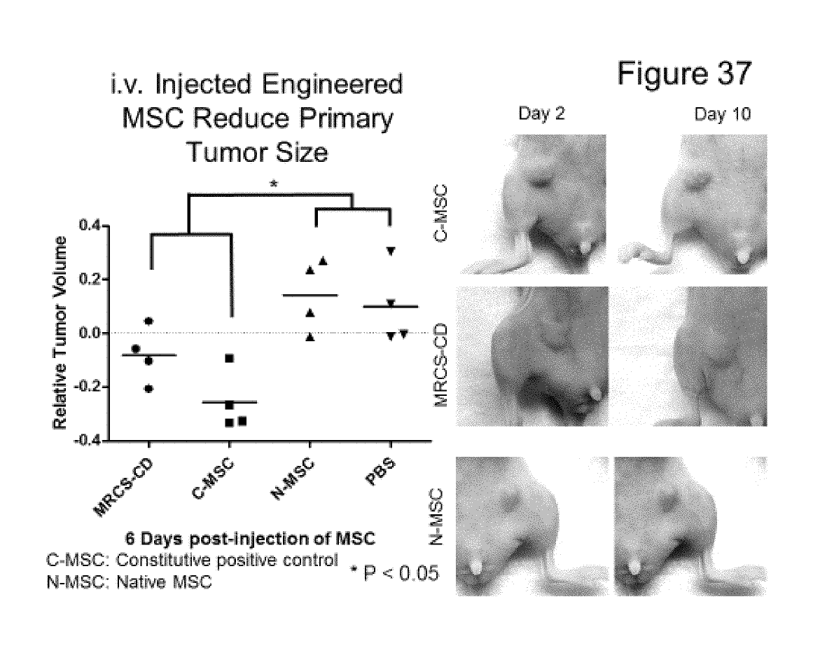

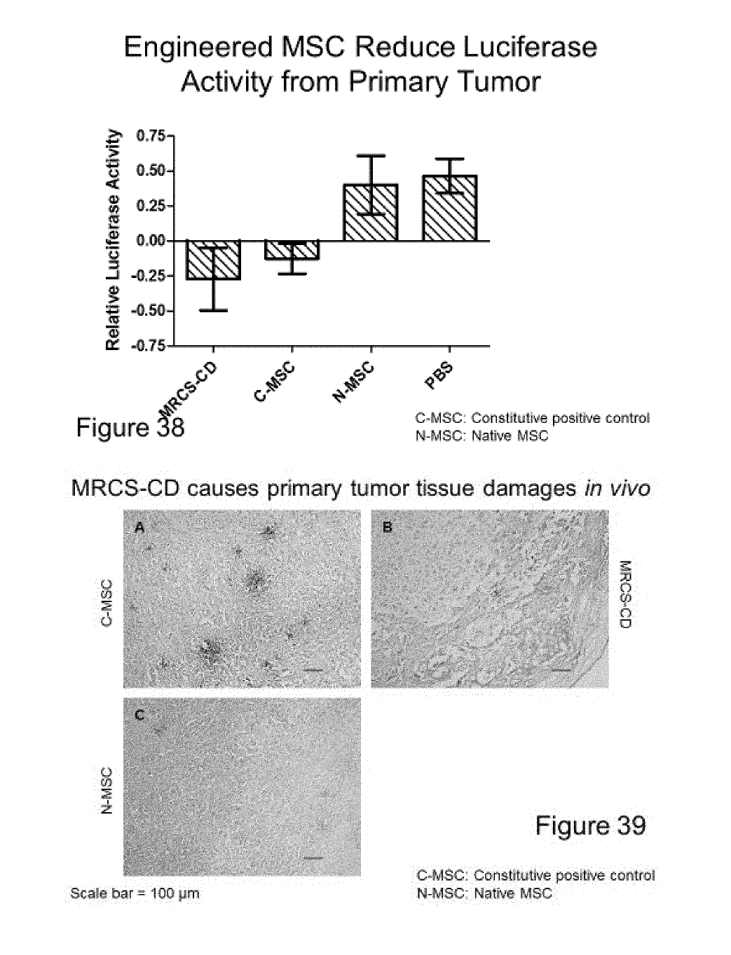

Tissue damage was assessed using TUNEL assay at Day 1 and Day 9. Increased brown HRP signal indicates increased damage to cell nuclei within lung tissues. Representative images of each treatment group and a healthy control from before and after treatment are shown in FIGS. 28 and 29. Quantification of TUNEL positive cells as a percentage of total cells shows a significant increase in tissue damage for C-MSC, and non-significant change in tissue damage for N-MSC and PBS (FIG. 30). MRCS-CD did cause lung damage over the course of treatment, but this damage was significantly lower than damage caused by C-MSC. Importantly, MRCS-CD caused no significant tissue damage when administered to tumor-free mice.

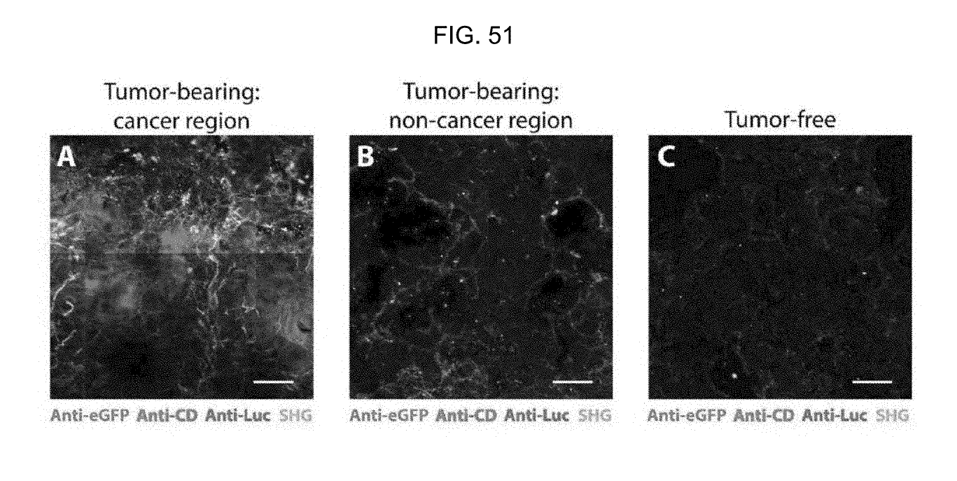

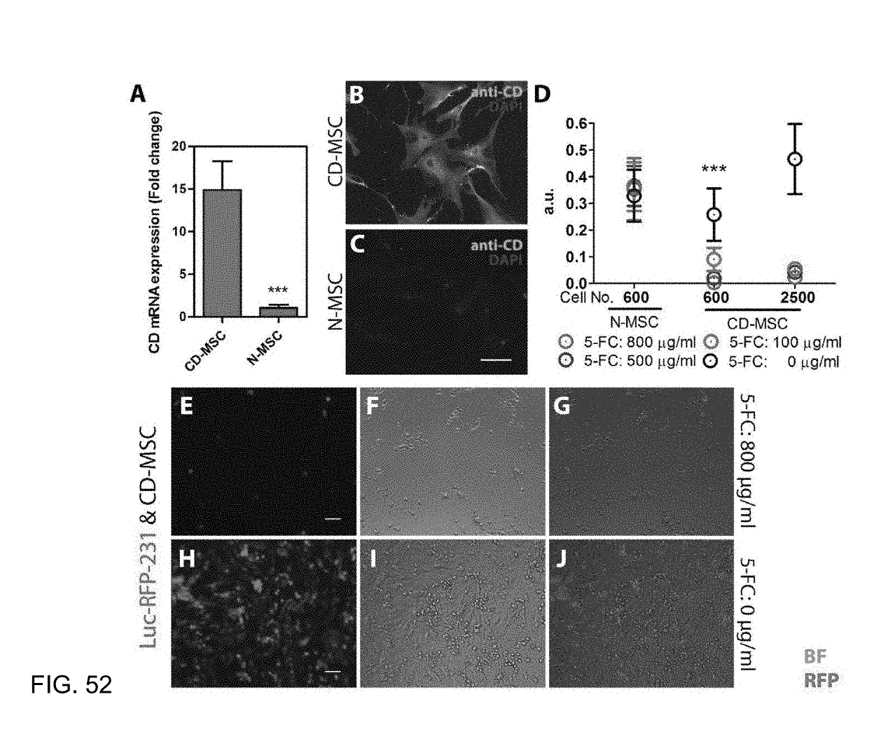

In order to further study how our MRCS interacts with the metastatic niche with the unique mechano-property, we co-transduced the MRCS-CD to constitutively express eGFP as a cell tracker. We then performed SHG imaging with ex vivo IHC staining 24 hours after the systemic infusion of MRCS to tumor-bearing (FIGS. 51A and 51B) and tumor-free (FIG. 51C) mice (Day 1). As observed on the SHG-IHC overlaid images, significantly more MRCS (characterized by the constitutively expressed eGFP) was observed in tumor-bearing lungs. Importantly, CD of eGFP-labeled MRCS-CD is preferentially activated in the cancer regions that are associated with more linearized collagen crosslinking (FIG. 51A). By contrast, few MRCS was activated to express CD in less linearized non-cancer regions (FIG. 51B) or in tumor-free lungs (FIG. 51C). Collectively, this set of data, together with our previous MRCS tumor homing data (FIG. 49 and FIG. 50) and MRCS-CD induced cell apoptosis at the metastatic sites (FIG. 27D), strongly suggest that the activation and tumor-killing functions of MRCS in vivo are specifically mediated by the unique, cancer associated mechano-cues.

### Discussion

Provided are therapeutic systems to directly interrogate matrix stiffness and applied it to localized delivery of agents to breast cancer metastases. This system has major clinical implications in increasing the effectiveness of therapies for the over 150,000 Americans living with metastatic breast cancer while also ameliorating the symptoms associated with systemic chemotherapy. Provided are MRCS for application to therapies targeting aberrant tissue stiffness in 1) both primary tumors and metastases in other organ systems (e.g., liver, brain and bone marrow) in breast cancer, and 2) other types of cancer and cancer metastases.

Provided are methods for preventing metastasis by targeting the “pre-metastatic niche” by either direct activation of a pro-drug at the pre-metastatic niche to destroy recruited bone marrow CD11b cells necessary for metastasis formation or by engineering the MRCS to secrete matrix remodeling enzymes such as metalloproteases to reduce the stiffness of the niche⁹. Importantly, MSC have been proven safe for transplantation in humans in many clinical trials, and has been approved for use in children with Graft-versus-host disease (GvHD) in Canada.

Provided are diagnostic tools to sensitively and selectively detect micrometastases especially at their early stages. Provided are methods using fluorescence and bioluminescence imaging. Systems can be imaged by, for example, positron emission tomography (PET) after integrating with HSV-1-tk reporter gene, to catalyze the phosphorylation of the thymidine analog [18F] FEAU. In this system, the phosphorylated form of [18F] FEAU accumulates inside of cells expressing the HSV-1-tk gene, facilitating imaging with PET^{[25]}. Provided are methods for guiding surgical interventions by highlighting areas of metastases or high invasive potential with the fluorescence reporter; a similar method using a systemically administered fluorescent probe has found utility in highlighting cancer cells for surgery^{[26]}.

### Advantages

Exemplary systems have major advantages over current techniques of imaging micrometastases in that they can amplify the signal from smaller numbers of cells by detecting the properties of the local microenvironment and that it can be used in vivo without a need for biopsy or invasive techniques. MRCS can be a routine practice for diagnosis of micrometastases and for monitoring treatment in high-risk patient groups. Furthermore, our system will provide a useful tool to study new biology of cancer metastasis and their interaction with the mechano-niche. For instance, exemplary methods comprising a stiffness “ruler” allow measurement and monitoring of stiffness in the tumor microenvironment in vivo, in real-time, in a dynamic fashion which is currently not possible. Such a system allows study how tumor cells re-model their mechano-niche in response to chemotherapy, which can develop new cancer therapeutics.

In nature, cells within tissues “feel” and sense mechano-environmental cues (e.g., forces, stiffness) and transduce that information to downstream functions such as invasion, migration or differentiation. Such mechano-niches play vital roles in development, hemostasis and disease progression including cancer, and therefore serve as an emerging target for next generation therapeutics. Matrix stiffness is an enormously appealing target for cancer therapeutics due to its long half-life (measured in years), making it refractory to development of resistance^{[14]}. Inspired by previous findings that 1) stiffness is highly increased in the metastatic niche, and 2) provided are methods effecting MSC differentiation to specific lineages depending on the stiffness of the microenvironment, provided are treatments for cancer metastasis by, for the first time, directly targeting the mechano-environmental cues of the metastatic niche. By using cells engineered to respond to variations in matrix stiffness provided are methods for detecting metastases at a higher resolution than current techniques such as CT or MRI, but also target metastases for localized activation of therapeutics.

Our system also takes advantage of the ability of MSC to specifically home to metastases. The natural ‘active’ homing (and the subsequent integration) ability of MSC to tumors and metastases enables the efficient delivery of ‘cargo’ to the target site. This circumvents many hurdles associated with the passive delivery (i.e., by direct administration or polymeric nanoparticles) including penetrating the endothelium, and the increased pressure associated with tumors. In particular, due to their small size, high dispersion to organs, and low vascularization, metastatic tumors may be less accessible to systemically infused chemotherapeutics or targeted nanoparticles. Such active and specific targeting combined with local and specific delivery of the pro-drug convertase/5-fluorocytosine system allows us to approach local therapeutic concentrations impossible with systemic infusion of chemotherapeutics with minimal side effects.

In addition, exemplary MRCS systems generate an entirely new technique to explore the native mechanical properties of tissues in vivo. Although previous studies have established that matrix stiffness is tightly linked to invasiveness and metastasis, current methods of measuring stiffness involve ex vivo measurements with atomic force microscopy (AFM) or compression devices. In addition, these techniques lack the resolution to directly measure the stiffness of the ECM with which the cells interact; instead, it measures the average stiffness of larger regions encompassing both ECM and cellular components of the tissues of interest. Provided is a cell-based, fluorescence “ruler” for measurement of matrix stiffness in situ that is a paradigm-shifting method of dynamically interrogating the mechano-environment of primary tumors, metastases, and changes in matrix stiffness during disease progression and response to therapies in vivo.

### Risks

A potential caveat is that MSC may, themselves, modify the local mechano-environment in vivo. Native MSC have previously been proposed to regulate cancer progression, both positively and negatively^{[15]}. We hypothesize, however, that even if this occurs the final differentiation status will still depend on and correlate with the initial properties of the metastatic niche. To mitigate this potential issue, we will explore the differentiation status of MSC over time and endeavor to use rapid time points at which specific reporter activation at the metastatic niche occurs^{[19]}. In fact, we reason that our MRCS may allow us in the future to study the roles of MSC in tumor progression, which is a subject of hot debate in the field^{[16]}. In particular, the tools we generate in the process of developing the MRCS (specifically promoter-driven reporters) will also be used to explore the differentiation status of exogenous and endogenous MSC in the primary tumor, metastases, and other organs over the course of cancer progression and therapeutic response.

Although several organs, including muscle (12 kPa) and bone (25-40 kPa) [4], approach or exceed the tissue stiffness of invasive breast cancer and may promote activation of our MRCS, we anticipate this will not be a major issue due to the inherent homing ability of MSC to cancer and metastases and their rapid clearance from non-inflamed or injured tissues^{[15, 16]}. Although MSC will encounter blood vessel endothelial cells, basement membrane and ECM components, each with their own characteristic stiffness, while in transit to the metastatic niche, we do not expect this to permanently influence reporter activity^{[4]}. In particular, many of these mechanical interactions involve shear stress, which does not regulate MSC differentiation. In addition, previous studies have established that expression of mechano-responsive genes is rapidly reversible¹⁶, which we will additionally characterize and optimize.

Although previous reports suggest the proximal 3 kBp upstream of each gene is sufficient to regulate their transcription, it is possible that response to other stimuli (such as hypoxia or inflammation) may interfere with mechano-specific activation. If it occurs, we will remove response elements to hypoxia and inflammation, such as HIF-1α and NF- [symbol] B consensus sequences, via well-established whole-plasmid site-directed mutagenesis. Our bioinformatics approach of identifying matrix stiffness-responsive promoter elements to generate synthetic promoters will also avoid the potential confounding effects of unwanted response elements in the native promoters.

Finally, the bystander effect of the CD/5-FC system may be too strong or weak to effectively treat metastases while sparing the normal lung. Multiple alternative methods exist to locally delivery therapeutics via transcriptional regulation of a promoter, including thymidine kinase, TRAIL, and IFN-β, which we will explore. Additionally, experiments applying our system in spontaneous, autochthonous models of breast cancer metastasis to the lung will be performed to fully validate the generalizability of our MRCS platform. Long-term survival studies, in which the primary tumor is removed following establishment of lung metastases and prior to treatment with the MRCS, will be performed to fully validate the MRCS as a realistic and viable treatment for clinical translation. In addition, as increased matrix stiffness is also associated with local invasion of the primary tumor and derangement of the vasculature; targeting these stiff areas of the primary tumor may both target the most invasive areas of the tumor and promote renormalization of the vasculature for treatments^{[7]}.

In alternative embodiments, stiffness sensing sequences: CACATTCCA (SEQ ID NO:1), are used, including e.g., a Minimal chicken TnT promoter (SEQ ID NO:2)

CACATTCCACACATTCCACTGCAAGCTTGAGACACATTCCACACAT TCCACTGCAAGCTTGGCCAGTGCCAAGTTGAGACACATTCCACACATTCC ACTGCAAGCTTGAGACACATTCCACACATTCCACTGCAAGCTTCTAGAGA TCTGCAGGTCGAGGTCGACGGTATCGATAAGCTTGGGGGTGGGCGCCGGG GGGACCTTAAAGCCTCTGCCCCCCAAGGAGCCCTTCCCAGACAGCCGCCG GCACCCACCGCTCCGTGGAC

LV-PL4 Promoterless Vector Cloning Protocol

1) Digest the promoterless vector (GenTarget, Inc. Cat # LV-PL4) with certain restriction enzyme for 1 hr;
2) Vector de-phosphorylation

a) Add 1/10 volume of 10× Antarctic Phosphatase Reaction Buffer to 1-5 μg of DNA cut with any restriction endonuclease in any buffer;

b) Add 1 μl of Antarctic Phosphatase (5 u) and mix;

c) Incubate for 15 min at 37° C. for 5′ extensions or blunt-ends;

d) Heat inactivate for 5 mins at 65° C.;

3) Run the digested/de-phosphorylated product in 1% agarose gel, cut the target band (7088 bp) and do gel purification. Store @ −20° C.;
4) Generation of insert fragments:
a) PCR amplification of insert or
b) Use certain restriction enzyme to cut off the insert;
   5) Use PCR purification or gel purification kit (step 3) to purify the fragments;

### 6) Ligation:

a) Use In-Fusion® HD Cloning Plus Kit (Clontech Laboratories Inc.) to fuse linearized promoterless vector from step 3 and insert fragments from step 5 @ 50° C. for 15 mins or
b) Use T4 Ligase to ligate purified linearized vectors and inserts;
   7) Transform the infusion product from step 6 into Stellar competent cells (Clontech Laboratories Inc.);
   8) Spread transformed cells onto Amp-LB plate and incubate overnight (16 hrs) @37° C.
   9) Pick up several colonies and culture overnight (16 hrs) in Amp-LB broth @ 37° C. and do colony PCR before mini-prep;
   10) Mini-prep and verify final plasmid by cutting with restriction enzymes and running 1% agarose gel for the size.

Lenti-Viral Transduction Protocol

11) 24 hrs before transfection, plate sufficient 293T cells (GenTarget Inc; cat #: TLV-C) (<P20) to achieve 80-90% confluence on the day of transfection (1×10⁷ (80% confluence) seeded on 10 cm petri-dish with 10 ml DMEM with 10% Fetal Bovine Serum (FBS) and 1% PenStrep (P/S));
12) 2 to 3 hrs before transfection, replace with fresh medium without P/S;

### 13) Transfection (Lipofectamine® LTX and PLUS™ Reagents)

a) Dilute the plasmid DNA shown below in 1.5 ml Opti-MEM/Reduced Serum Medium. Mix thoroughly by pipetting up and down twice:

| | | | |
|---|---|---|---|
| Vector | Amount | Size (kb) | For 10 cm dish |

| | | | | |
|---|---|---|---|---|
| *Transfer plasmid | 10 | μg | 8.1-8.5 | vector genome |
| pMDLg/pRRE | 5 | μg | 8.8 | HIV-1 GAG/POL |
| (Addgene: 12251) | | | | |
| pRSV/REV | 5 | μg | 4.1 | HIV-1 REV |
| (Addgene: 12253) | | | | |
| pMD2.G | 3.5 | μg | 5.8 | VSV glycoprotein |
| (Addgene: 12259) | | | | |
| Total | 23.5 | μg | | |

*Transfer plasmid = promoters + CDS inserted into promoterless vector (GenTarget Inc; cat#: LV-PL4)

b) Mix PLUS™ Reagent gently before use, add 15.5 ul PLUS™ Reagent directly to the diluted DNA (Reagent A). Mix gently and incubate for 5 mins @ RT;
c) Add 31 μl Lipofectamine® LTX into 1.5 ml Opti-MEM® Reduced Serum Medium (Reagent B). Mix gently. Proceed to the next step within 5 mins;
d) Mix Reagent A and B gently, and incubate for 30 mins @ room temperature;
e) Aspirate the medium from 293T cells and rinse with 5 ml Opti-MEM® Reduced Serum Medium twice;
f) Add Reagent mixture onto 293T cells and mix gently;
g) Incubate @ 37° C. in a CO₂ incubator for 4 hrs and add 3 ml P/S free-DMEM with 20% FBS;
h) Incubate overnight (16 hrs) and replace the medium with 6 ml P/S free-DMEM with 10% FBS;
   14) Triplicate MSC in 6-well plate with a confluency of 70 to 80%;
   15) Harvest the virus:
a) Collect the supernatant of 293T cells 48 hrs after step 3 h (1^{st} batch);
b) Transfer the supernatant into a 10 ml syringe with 0.45 um filter and let the supernatant go through the filter;
c) Add 120 μl 5 mg/ml protamine sulfate (in DMEM, sterile filtered) to make a final concentration of 100 μg/ml;
d) Refill the petri-dish with 5 ml fresh P/S-free DMEM;
e) 72 hrs after step 3 h, repeat the virus harvest step 5a-c;
f) Aspirate virus and replace with fresh medium, and incubate for 6 hrs before selection with 10 μg/ml puromycin for 48 hrs and then maintain engineered cells with 1 to 2 g/ml puromycin afterwards;
   6) Virus can be store @ −80 C if necessary.

### Transfer Plasmids:

LV-PL4-CMV::CD

CMV enhancer+promoter: pcDNA3.1(+)/Luc2=tdT, Addgene 43904

Infusion primers:
(SEQ ID NO: 3) forward (Fw):
ggtggtggatccTGTACGGGCCAGATATACGC
(SEQ ID NO: 4) reverse (Rv):
ggtggtggatccGCCAGCTTGGGTCTCCCTAT
FCY::FUR (fused cytosine deaminase (CD)): pSelect-
zeo-Fcy::Fur, InvivoGen, Inc. Cat#: psetz-fcyfur
Infusion primers:
(SEQ ID NO: 5) Fw:
ggtggtatcgatgccaccATGGTCACAGGAGGC
(SEQ ID NO: 6) Rv:
ggtggtatcgatAGCTAGCTCAGGTTTAGACACAGTAG
Promoterless vector (GenTarget, Inc Cat# LV-PL4)
(SEQ ID NO: 7)
ggatccTGTACGGGCCAGATATACGCGTTGACATTGATTATTGACTAGTT
ATTAATAGTAATCAATTACGGGGTCATTAGTTCATAGCCCATATATGGAG
TTCCGCGTTACATAACTTACGGTAAATGGCCCGCCTGGCTGACCGCCCAA
CGACCCCCGCCCATTGACGTCAATAATGACGTATGTTCCCATAGTAACGC
CAATAGGGACTTTCCATTGACGTCAATGGGTGGAGTATTTACGGTAAACT
GCCCACTTGGCAGTACATCAAGTGTATCATATGCCAAGTACGCCCCCTAT
TGACGTCAATGACGGTAAATGGCCCGCCTGGCATTATGCCCAGTACATGA
CCTTATGGGACTTTCCTACTTGGCAGTACATCTACGTATTAGTCATCGCT
ATTACCATGGTGATGCGGTTTTGGCAGTACATCAATGGGCGTGGATAGCG
GTTTGACTCACGGGGATTTCCAAGTCTCCACCCCATTGACGTCAATGGGA
GTTTGTTTTGGCACCAAAATCAACGGGACTTTCCAAAATGTCGTAACAAC
TCCGCCCCATTGACGCAAATGGGCGGTAGGCGTGTACGGTGGGAGGTCTA
TATAAGCAGAGCTCTCTGGCTAACTAGAGAACCCACTGCTTACTGGCTTA
TCGAAATTAATACGACTCACTATAGGGAGACCCAAGCTGGCgGATCCGTA
TACATcgatgccaccATGGTCACAGGAGGCATGGCTTCAAAGTGGGACCA
GAAGGGCATGGACATTGCCTATGAGGAGGCTGCTCTGGGCTACAAGGAGG
GAGGGGTCCCAATTGGTGGCTGCCTCATCAACAACAAGGATGGCAGTGTC
CTGGGCAGGGGCCACAACATGAGGTTCCAGAAGGGCAGTGCCACCCTGCA
TGGGGAGATCAGCACCCTGGAGAACTGTGGCAGGCTGGAGGGCAAGGTCT
ACAAGGACACCACTCTGTACACCACCCTCAGCCCTTGTGACATGTGCACA
GGGGCCATCATCATGTATGGCATTCCCAGGTGTGTGGTGGGAGAGAATGT
CAACTTCAAGTCAAAAGGAGAGAAGTACCTCCAGACCAGGGGCCATGAGG
TGGTTGTGGTGGATGATGAGAGGTGCAAGAAGATTATGAAGCAGTTCATT
GATGAGAGACCCCAGGACTGGTTTGAGGACATTGGGGAGGCCTCTGAGCC
CTTCAAGAATGTGTACCTCCTCCCCCAGACCAACCAACTCCTGGGACTCT
ACACCATCATCAGGAACAAGAACACCACCAGGCCAGACTTCATCTTCTAC
AGTGACAGGATCATCAGGCTCCTGGTGGAGGAGGGCCTCAACCACCTCCC
TGTGCAGAAGCAGATTGTGGAGACTGACACCAATGAGAACTTTGAGGGAG
TGTCTTTCATGGGCAAGATTTGTGGGGTGTCCATTGTGAGGGCTGGGGAG
AGCATGGAGCAGGGCCTGAGGGACTGTTGCAGGAGTGTGAGGATTGGCAA
GATCCTGATCCAGAGGGATGAGGAGACTGCCCTGCCCAAGCTGTTCTATG
AGAAGCTCCCTGAAGACATCTCTGAGAGGTATGTCTTCCTCCTGGACCCC
ATGCTGGCAACTGGAGGCTCTGCAATCATGGCCACTGAGGTGCTCATCAA
GAGGGGAGTCAAGCCTGAGAGGATCTACTTCCTCAACCTCATCTGCTCAA
AGGAGGGCATTGAGAAGTACCATGCTGCCTTCCCTGAAGTGAGGATTGTC
ACTGGGGCTCTGGACAGGGGCCTGGATGAGAACAAGTACCTGGTCCCTGG
CCTGGGAGACTTTGGGGACAGATACTACTGTGTCTAAACCTGAGCTAGCT
atcgatgggcccactagtgtcgacgctagctctagatgtacaaagtggtg
ctagcactctcagtacaatctgctctgatgccgcatagttaagccagtat
ctgctccctgcttgtgtgaggaggtcgctgagtagtgcgcgagcaaaatt
taagctacaacaaggcaaggcttgaccgacaattgcatgaagaatctgct
tagggttaggcgttagcgctgcttcgcgatgtacgggccagatatacgcg
tatctgaggggactagggtgtgtttaggcgaaaagcggggcttcggttgt
acgcggttaggagtcccctcaggatatagtagtttcgcttttgcataggg
agggggaaatgtagtcttatgcaatactcttgtagtcttgcaacatggta
acgatgagttagcaacatgccttacaaggagagaaaaagcaccgtgcatg
ccgattggtggaagtaaggtggtacgatcgtgccttattaggaaggcaac
agacgggtctgacatggattggacgaaccactgaattccgcattgcagag
atattgtatttaagtgcctagctcgatacaataaacgccatttgaccatt
caccacattggtgtgcacctccaaagcgctcaccatgaccgagtacaagc
ccacggtgcgcctcgccacccgcgacgacgtccccagggccgtacgcacc
ctcgccgccgcgttcgccgactaccccgccacgcgccacaccgtcgatcc
ggaccgccacatcgagcgggtcaccgagctgcaagaactcttcctcacgc
gcgtcgggctcgacatcggcaaggtgtgggtcgcggacgacggcgccgcg
gtggcggtctggaccacgccggagagcgtcgaagcgggggcggtgttcgc
cgagatcggcccgcgcatggccgagttgagcggttcccggctggccgcgc
agcaacagatggaaggcctcctggcgccgcaccggcccaaggagcccgcg
tggttcctggccaccgtcggcgtctcgcccgaccaccagggcaagggtct
gggcagcgccgtcgtgctccccggagtggaggcggccgagcgcgccgggg
tgcccgccttcctggagacctccgcgccccgcaacctccccttctacgag
cggctcggcttcaccgtcaccgccgacgtcgaggtgcccgaaggaccgcg
cacctggtgcatgacccgcaagcccggtgcctgaactagttaggtttaaa
cacgcgtaccggttagtaatgatcgacaatcaacctctggattacaaaat
ttgtgaaagattgactggtattcttaactatgttgctccttttacgctat
gtggatacgctgctttaatgcctttgtatcatgctattgcttcccgtatg
gctttcattttctcctccttgtataaatcctggttgctgtctctttatga
ggagttgtggcccgttgtcaggcaacgtggcgtggtgtgcactgtgtttg
ctgacgcaacccccactggttggggcattgccaccacctgtcagctcctt
tccgggactttcgctttccccctccctattgccacggcggaactcatcgc
cgcctgccttgcccgctgctggacaggggctcggctgttgggcactgaca
attccgtggtgttgtcggggaagctgacgtcctttccatggctgctcgcc
tgtgttgccacctggattctgcgcgggacgtccttctgctacgtcccttc
ggccctcaatccagcggaccttccttcccgcggcctgctgccggctctgc
ggcctcttccgcgtcttcgccttcgccctcagacgagtcggatctccctt
tgggccgcctccccgcctggcgatggtacctttaagaccaatgacttaca
aggcagctgtagatcttagccactttttaaaagaaaaggggggactggaa
gggctaattcactcccaacgaagacaagatctgctttttgcttgtactgg
gtctctctggttagaccagatctgagcctgggagctctctggctaactag
ggaacccactgcttaagcctcaataaagcttgccttgagtgcttcaagta
gtgtgtgcccgtctgttgtgtgactctggtaactagagatccctcagacc
cttttagtcagtgtggaaaatctctagcagtagtagttcatgtcatctta
ttattcagtatttataacttgcaaagaaatgaatatcagagagtgagagg
aacttgtttattgcagcttataatggttacaaataaagcaatagcatcac
aaatttcacaaataaagcatttttttcactgcattctagagtggtagtcc
aaactcatcaatgtatcttatcatgtctggctctagctatcccgccccta
actccgcccatcccgcccctaactccgcccagttccgcccattctccgcc
ccatggctgactaattttttttatttatgcagaggccgaggccgcctcgg
cctctgagctattccagaagtagtgaggaggcttttttggaggcctaggg
acgtacccaattcgccctatagtgagtcgtattacgcgcgctcactggcc
gtcgttttacaacgtcgtgactgggaaaaccctggcgttacccaacttaa
tcgccttgcagcacatccccctttcgccagctggcgtaatagcgaagagg
cccgcaccgatcgcccttcccaacagttgcgcagcctgaatggcgaatgg
gacgcgccctgtagcggcgcattaagcgcggcgggtgtggtggttacgcg
cagcgtgaccgctacacttgccagcgccctagcgcccgctcctttcgctt
tcttcccttcctttctcgccacgttcgccggctttcccgtcaagctctaa
atcgggggctccctttagggttccgatttagtgctttacggcacctcgac
cccaaaaaacttgattagggtgatggttcacgtagtgggccatcgccctg
atagacggtttttcgccctagacgaggagtccacgttctttaatagtgga
ctcttgaccaaactggaacaacactcaaccctatctcggtctattctttt
gatttataagggattttgccgatttcggcctattggttaaaaaatgagct
gatttaacaaaaatttaacgcgaattttaacaaaatattaacgcttacaa
tttaggtggcacttacggggaaatgtgcgcggaacccctatagtttattt
ttctaaatacattcaaatatgtatccgctcatgagacaataaccctgata
aatgcttcaataatattgaaaaaggaagagtatgagtattcaacatttcc
gtgtcgcccttattcccttttttgcggcattagccttcctgtttagctca
cccagaaacgctggtgaaagtaaaagatgctgaagatcagttgggtgcac
gagtgggttacatcgaactggatctcaacagcggtaagatccttgagagt
tttcgccccgaagaacgttttccaatgatgagcacttttaaagttctgct
atgtggcgcggtattatcccgtattgacgccgggcaagagcaactcggtc
gccgcatacactattctcagaatgacttggttgagtactcaccagtcaca
gaaaagcatcttacggatggcatgacagtaagagaattatgcagtgctgc
cataaccatgagtgataacactgcggccaacttacttctgacaacgatcg
gaggaccgaaggagctaaccgcttttttgcacaacatgggggatcatgta
actcgccttgatcgttgggaaccggagctgaatgaagccataccaaacga
cgagcgtgacaccacgatgcctgtagcaatggcaacaacgttgcgcaaac
tattaactggcgaactacttactctagcttcccggcaacaattaatagac
tggatggaggcggataaagagcaggaccacttctgcgctcggcccttccg
gctggctggtttattgctgataaatctggagccggtgagcgtgggtctcg
cggtatcattgcagcactggggccagatggtaagccctcccgtatcgtag
ttatctacacgacggggagtcaggcaactatggatgaacgaaatagacag
atcgctgagataggtgcctcactgattaagcattggtaactgtcagacca
agtttactcatatatactttagattgatttaaaacttcatttttaattta
aaaggatctaggtgaagatcctttttgataatctcatgaccaaaatccct
taacgtgagttacgttccactgagcgtcagaccccgtagaaaagatcaaa
ggatcttcttgagatcctttttttctgcgcgtaatctgctgcttgcaaac
aaaaaaaccaccgctaccagcggtggtagtagccggatcaagagctacca
actctttttccgaaggtaactggcttcagcagagcgcagataccaaatac
tgttcttctagtgtagccgtagttaggccaccacttcaagaactctgtag
caccgcctacatacctcgctctgctaatcctgttaccagtggctgctgcc
agtggcgataagtcgtgtcttaccgggttggactcaagacgatagttacc
ggataaggcgcagcggtcgggctgaacggggggttcgtgcacacagccca
gcttggagcgaacgacctacaccgaactgagatacctacagcgtgagcta
tgagaaagcgccacgcttcccgaagggagaaaggcggacaggtatccggt
aagcggcagggtcggaacaggagagcgcacgagggagcttccagggggaa
acgcctggtatctttatagtcctgtcgggtacgccacctctgacttgagc
gtcgatttttgtgatgctcgtcaggggggcggagcctatggaaaaacgcc
agcaacgcggcctttttacggacctggccttagctggccttagctcacat
gactacctgcgttatcccctgattctgtggataaccgtattaccgccttt
gagtgagctgataccgctcgccgcagccgaacgaccgagcgcagcgagtc
agtgagcgaggaagctggaagggctaattcactcccaaagaagacaagat
atccttgatctgtggatctaccacacacaaggctacttccctgattagca
gaactacacaccagggccaggggtcagatatccactgacctttggatggt
gctacaagctagtaccagttgagccagataaggtagaagaggccaataaa
ggagagaacaccagcttgttacaccctgtgagcctgcatgggatggatga
cccggagagagaagtgttagagtggaggtttgacagccgcctagcatttc
atcacgtggcccgagagctgcatccggagtacttcaagaactgctgatat
cgagcttgctacaagggactttccgctggggactttccagggaggcgtgg
cctgggcgggactggggagtggcgagccctcagatcctgcatataagcag
ctgctttttgcctgtactgggtctctctggttagaccagatctgagcctg
ggagctctctggctaactagggaacccactgcttaagcctcaataaagct
tgccttgagtgcttcaagtagtgtgtgcccgtctgagtgtgactctggta
actagagatccctcagacccttttagtcagtgtggaaaatctctagcagt
ggcgcccgaacagggacttgaaagcgaaagggaaaccagaggagctctct
cgacgcaggactcggcttgctgaagcgcgcacggcaagaggcgaggggcg
gcgactggtgagtacgccaaaaattttgactagcggaggctagaaggaga
gagatgggtgcgagagcgtcagtattaagcgggggagaattagatcgcga
tgggaaaaaattcggttaaggccagggggaaagaaaaaatataaattaaa
acatatagtatgggcaagcagggagctagaacgattcgcagttaatcctg
gcctgttagaaacatcagaaggctgtagacaaatactgggacagctacaa
ccatcccttcagacaggatcagaagaacttagatcattatataatacagt
agcaaccctctattgtgtgcatcaaaggatagagataaaagacaccaagg
aagctttagacaagatagaggaagagcaaaacaaaagtaagaccaccgca
cagcaagcggccgctgatcttcagacctggaggaggagatatgagggaca
attggagaagtgaattatataaatataaagtagtaaaaattgaaccatta
ggagtagcacccaccaaggcaaagagaagagtggtgcagagagaaaaaag
agcagtgggaataggagctttgttccttgggttcttgggagcagcaggaa
gcactatgggcgcagcgtcaatgacgctgacggtacaggccagacaatta
ttgtctggtatagtgcagcagcagaacaatttgctgagggctattgaggc
gcaacagcatctgttgcaactcacagtctggggcatcaagcagctccagg
caagaatcctggctgtggaaagatacctaaaggatcaacagctcctgggg
atttggggttgctctggaaaactcatttgcaccactgctgtgccttggaa
tgctagttggagtaataaatctctggaacagataggaatcacacgacctg
gatggagtgggacagagaaattaacaattacacaagcttaatacactcct
taattgaagaatcgcaaaaccagcaagaaaagaatgaacaagaattattg
gaattagataaatgggcaagtttgtggaattggtttaacataacaaattg
gctgtggtatataaaattattcataatgatagtaggaggcttggtaggtt
taagaatagtttttgctgtactttctatagtgaatagagttaggcaggga
tattcaccattatcgtttcagacccacctcccaaccccgaggggacccga
caggcccgaaggaatagaagaagaaggtggagagagagacagagacagat
ccattcgattagtgaacggatctcgacggtatcggttaacttttaaaaga
aaaggggggattggggggtacagtgcaggggaaagaatagtagacataat
agcaacagacatacaaactaaagaattacaaaaacaaattacaaaaattc
aaaattttatcg

LV-PL4-GTIIC::CD

GTIIC stiffness sensing promoter: Addgene 34615: 8×GTIIC-luciferase (Dupont, Nature, 2011)

Infusion primers:
(SEQ ID NO: 8) Fw:
AATTTTATCGGGATCCCGAGCTCTTACGCGTGCTA
(SEQ ID NO: 9) Rv:
CGATGTATACGGATCCtttatATCGTCCCACGGAGCG
FCY::FUR (fused cytosine deaminase (CD)): pSelect-
zeo-Fcy::Fur, InvivoGen, Inc. Cat#: psetz-fcyfur
Infusion primers:
(SEQ ID NO: 10) Fw:
ggtggtatcgatgccaccATGGTCACAGGAGGC
(SEQ ID NO: 11) Rv:
ggtggtatcgatAGCTAGCTCAGGTTTAGACACAGTAG
Promoterless vector (GenTarget, Inc Cat# LV-PL4)
(SEQ ID NO: 12)
ggatccCGAGCTCTTACGCGTGCTAGCCCGGGCTAGCCCGGCCAGTGCCA
AGTTGAGACACATTCCACACATTCCACTGCAAGCTTGAGACACATTCCAC
ACATTCCACTGCAAGCTTGGCCAGTGCCAAGTTGAGACACATTCCACACA
TTCCACTGCAACTTGAGACACATTCCACACATTCCACTGCAAGCTTCTAG
AGATCTGCAGGTCGAGGTCGACGGTATCGATAAGCTTGGGGGTGGGCGCC
GGGGGGACCTTAAAGCCTCTGCCCCCCAAGGAGCCCTTCCCAGACAGCCG
CCGGCACCCACCGCTCCGTGGGACGATataaagGATCCGTATACATcgat
gccaccATGGTCACAGGAGGCATGGCTTCAAAGTGGGACCAGAAGGGCAT
GGACATTGCCTATGAGGAGGCTGCTCTGGGCTACAAGGAGGGAGGGGTCC
CAATTGGTGGCTGCCTCATCAACAACAAGGATGGCAGTGTCCTGGGCAGG
GGCCACAACATGAGGTTCCAGAAGGGCAGTGCCACCCTGCATGGGGAGAT
CAGCACCCTGGAGAACTGTGGCAGGCTGGAGGGCAAGGTCTACAAGGACA
CCACTCTGTACACCACCCTCAGCCCTTGTGACATGTGCACAGGGGCCATC
ATCATGTATGGCATTCCCAGGTGTGTGGTGGGAGAGAATGTCAACTTCAA
GTCAAAAGGAGAGAAGTACCTCCAGACCAGGGGCCATGAGGTGGTTGTGG
TGGATGATGAGAGGTGCAAGAAGATTATGAAGCAGTTCATTGATGAGAGA
CCCCAGGACTGGTTTGAGGACATTGGGGAGGCCTCTGAGCCCTTCAAGAA
TGTGTACCTCCTCCCCCAGACCAACCAACTCCTGGGACTCTACACCATCA
TCAGGAACAAGAACACCACCAGGCCAGACTTCATCTTCTACAGTGACAGG
ATCATCAGGCTCCTGGTGGAGGAGGGCCTCAACCACCTCCCTGTGCAGAA
GCAGATTGTGGAGACTGACACCAATGAGAACTTTGAGGGAGTGTCTTTCA
TGGGCAAGATTTGTGGGGTGTCCATTGTGAGGGCTGGGGAGAGCATGGAG
CAGGGCCTGAGGGACTGTTGCAGGAGTGTGAGGATTGGCAAGATCCTGAT
CCAGAGGGATGAGGAGACTGCCCTGCCCAAGCTGTTCTATGAGAAGCTCC
CTGAAGACATCTCTGAGAGGTATGTCTTCCTCCTGGACCCCATGCTGGCA
ACTGGAGGCTCTGCAATCATGGCCACTGAGGTGCTCATCAAGAGGGGAGT
CAAGCCTGAGAGGATCTACTTCCTCAACCTCATCTGCTCAAAGGAGGGCA
TTGAGAAGTACCATGCTGCCTTCCCTGAAGTGAGGATTGTCACTGGGGCT
CTGGACAGGGGCCTGGATGAGAACAAGTACCTGGTCCCTGGCCTGGGAGA
CTTTGGGGACAGATACTACTGTGTCTAAACCTGAGCTAGCTatcgatggg
cccactagtgtcgacgctagctctagatgtacaaagtggtgctagcactc
tcagtacaatctgctctgatgccgcatagttaagccagtatctgctccct
gcttgtgtgttggaggtcgctgagtagtgcgcgagcaaaatttaagctac
aacaaggcaaggcttgaccgacaattgcatgaagaatctgcttagggtta
ggcgttttgcgctgcttcgcgatgtacgggccagatatacgcgtatctga
ggggactagggtgtgtttaggcgaaaagcggggcttcggttgtacgcggt
taggagtcccctcaggatatagtagtttcgcttttgcatagggaggggga
aatgtagtcttatgcaatactcttgtagtcttgcaacatggtaacgatga
gttagcaacatgccttacaaggagagaaaaagcaccgtgcatgccgattg
gtggaagtaaggtggtacgatcgtgccttattaggaaggcaacagacggg
tctgacatggattggacgaaccactgaattccgcattgcagagatattgt
atttaagtgcctagctcgatacaataaacgccatttgaccattcaccaca
ttggtgtgcacctccaaagcgctcaccatgaccgagtacaagcccacggt
gcgcctcgccacccgcgacgacgtccccagggccgtacgcaccctcgccg
ccgcgttcgccgactaccccgccacgcgccacaccgtcgatccggaccgc
cacatcgagcgggtcaccgagctgcaagaactcttcctcacgcgcgtcgg
gctcgacatcggcaaggtgtgggtcgcggacgacggcgccgcggtggcgg
tctggaccacgccggagagcgtcgaagcgggggcggtgttcgccgagatc
ggcccgcgcatggccgagttgagcggacccggctggccgcgcagcaacag
atggaaggcctcctggcgccgcaccggcccaaggagcccgcgtggttcct
ggccaccgtcggcgtctcgcccgaccaccagggcaagggtctgggcagcg
ccgtcgtgctccccggagtggaggcggccgagcgcgccggggtgcccgcc
ttcctggagacctccgcgccccgcaacctccccttctacgagcggctcgg
cttcaccgtcaccgccgacgtcgaggtgcccgaaggaccgcgcacctggt
gcatgacccgcaagcccggtgcctgaactagttaggtttaaacacgcgta
ccggttagtaatgatcgacaatcaacctctggattacaaaatagtgaaag
attgactggtattcttaactatgagctcctatacgctatgtggatacgct
gctttaatgcctagtatcatgctattgcttcccgtatggctttcattttc
tcctccttgtataaatcctggttgctgtctctttatgaggagttgtggcc
cgttgtcaggcaacgtggcgtggtgtgcactgtgtttgctgacgcaaccc
ccactggttggggcattgccaccacctgtcagctcctttccgggactttc
gctttccccctccctattgccacggcggaactcatcgccgcctgccttgc
ccgctgctggacaggggctcggctgttgggcactgacaattccgtggtgt
tgtcggggaagctgacgtcctttccatggctgctcgcctgtgttgccacc
tggattctgcgcgggacgtccttctgctacgtcccttcggccctcaatcc
agcggaccttccttcccgcggcctgctgccggctctgcggcctcttccgc
gtcttcgccttcgccctcagacgagtcggatctccctagggccgcctccc
cgcctggcgatggtacctttaagaccaatgacttacaaggcagctgtaga
tcttagccactattaaaagaaaaggggggactggaagggctaattcactc
ccaacgaagacaagatctgctattgcttgtactgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtagtagttcatgtcatcttattattcagtatttat
aacttgcaaagaaatgaatatcagagagtgagaggaacttgtttattgca
gcttataatggttacaaataaagcaatagcatcacaaatttcacaaataa
agcattatttcactgcattctagttgtggtagtccaaactcatcaatgta
tcttatcatgtctggctctagctatcccgcccctaactccgcccatcccg
cccctaactccgcccagaccgcccattctccgccccatggctgactaatt
tatttatttatgcagaggccgaggccgcctcggcctctgagctattccag
aagtagtgaggaggctataggaggcctagggacgtacccaattcgcccta
tagtgagtcgtattacgcgcgctcactggccgtcgttttacaacgtcgtg
actgggaaaaccctggcgttacccaacttaatcgccttgcagcacatccc
cctttcgccagctggcgtaatagcgaagaggcccgcaccgatcgcccttc
ccaacagttgcgcagcctgaatggcgaatgggacgcgccctgtagcggcg
cattaagcgcggcgggtgtggtggttacgcgcagcgtgaccgctacactt
gccagcgccctagcgcccgctcctttcgctttcttcccttcctttctcgc
cacgttcgccggctttccccgtcaagctctaaatcgggggctccctttag
ggttccgatttagtgctttacggcacctcgaccccaaaaaacttgattag
ggtgatggacacgtagtgggccatcgccctgatagacggtattcgcccta
gacgttggagtccacgttctttaatagtggactcttgttccaaactggaa
caacactcaaccctatctcggtctattcttttgatttataagggattttg
ccgatttcggcctattggttaaaaaatgagctgatttaacaaaaatttaa
cgcgaattttaacaaaatattaacgcttacaatttaggtggcacttacgg
ggaaatgtgcgcggaacccctatagtttattatctaaatacattcaaata
tgtatccgctcatgagacaataaccctgataaatgcttcaataatattga
aaaaggaagagtatgagtattcaacataccgtgtcgcccttattccatat
tgcggcattagccttcctgtttagctcacccagaaacgctggtgaaagta
aaagatgctgaagatcagagggtgcacgagtgggttacatcgaactggat
ctcaacagcggtaagatccttgagagttttcgccccgaagaacgttttcc
aatgatgagcacttttaaagttctgctatgtggcgcggtattatcccgta
ttgacgccgggcaagagcaactcggtcgccgcatacactattctcagaat
gacttggttgagtactcaccagtcacagaaaagcatcttacggatggcat
gacagtaagagaattatgcagtgctgccataaccatgagtgataacactg
cggccaacttacttctgacaacgatcggaggaccgaaggagctaaccgct
tttttgcacaacatgggggatcatgtaactcgccttgatcgttgggaacc
ggagctgaatgaagccataccaaacgacgagcgtgacaccacgatgcctg
tagcaatggcaacaacgagcgcaaactattaactggcgaactacttactc
tagcttcccggcaacaattaatagactggatggaggcggataaagttgca
ggaccacttctgcgctcggcccttccggctggctggtttattgctgataa
atctggagccggtgagcgtgggtctcgcggtatcattgcagcactggggc
cagatggtaagccctcccgtatcgtagttatctacacgacggggagtcag
gcaactatggatgaacgaaatagacagatcgctgagataggtgcctcact
gattaagcattggtaactgtcagaccaagtttactcatatatactttaga
ttgatttaaaacttcatttttaatttaaaaggatctaggtgaagatcctt
tttgataatctcatgaccaaaatcccttaacgtgagttacgttccactga
gcgtcagaccccgtagaaaagatcaaaggatcttcttgagatcctttttt
tctgcgcgtaatctgctgcttgcaaacaaaaaaaccaccgctaccagcgg
tggtagtttgccggatcaagagctaccaactctttttccgaaggtaactg
gcttcagcagagcgcagataccaaatactgttcttctagtgtagccgtag
ttaggccaccacttcaagaactctgtagcaccgcctacatacctcgctct
gctaatcctgttaccagtggctgctgccagtggcgataagtcgtgtctta
ccgggttggactcaagacgatagttaccggataaggcgcagcggtcgggc
tgaacggggggttcgtgcacacagcccagcttggagcgaacgacctacac
cgaactgagatacctacagcgtgagctatgagaaagcgccacgcttcccg
aagggagaaaggcggacaggtatccggtaagcggcagggtcggaacagga
gagcgcacgagggagcttccagggggaaacgcctggtatctttatagtcc
tgtcgggtttcgccacctctgacttgagcgtcgatttttgtgatgctcgt
caggggggcggagcctatggaaaaacgccagcaacgcggcctttttacgg
acctggccttagctggccttagctcacatgttctttcctgcgttatcccc
tgattctgtggataaccgtattaccgcctttgagtgagctgataccgctc
gccgcagccgaacgaccgagcgcagcgagtcagtgagcgaggaagctgga
agggctaattcactcccaaagaagacaagatatccttgatctgtggatct
accacacacaaggctacttccctgattagcagaactacacaccagggcca
ggggtcagatatccactgacctttggatggtgctacaagctagtaccagt
tgagccagataaggtagaagaggccaataaaggagagaacaccagcttgt
tacaccctgtgagcctgcatgggatggatgacccggagagagaagtgtta
gagtggaggtttgacagccgcctagcatttcatcacgtggcccgagagct
gcatccggagtacttcaagaactgctgatatcgagcttgctacaagggac
tttccgctggggactttccagggaggcgtggcctgggcgggactggggag
tggcgagccctcagatcctgcatataagcagctgctttttgcctgtactg
ggtctctctggttagaccagatctgagcctgggagctctctggctaacta
gggaacccactgcttaagcctcaataaagcttgccttgagtgcttcaagt
agtgtgtgcccgtctgagtgtgactctggtaactagagatccctcagacc
cttttagtcagtgtggaaaatctctagcagtggcgcccgaacagggactt
gaaagcgaaagggaaaccagaggagctctctcgacgcaggactcggcttg
ctgaagcgcgcacggcaagaggcgaggggcggcgactggtgagtacgcca
aaaattttgactagcggaggctagaaggagagagatgggtgcgagagcgt
cagtattaagcgggggagaattagatcgcgatgggaaaaaattcggttaa
ggccagggggaaagaaaaaatataaattaaaacatatagtatgggcaagc
agggagctagaacgattcgcagttaatcctggcctgttagaaacatcaga
aggctgtagacaaatactgggacagctacaaccatcccttcagacaggat
cagaagaacttagatcattatataatacagtagcaaccctctattgtgtg
catcaaaggatagagataaaagacaccaaggaagctttagacaagataga
ggaagagcaaaacaaaagtaagaccaccgcacagcaagcggccgctgatc
ttcagacctggaggaggagatatgagggacaattggagaagtgaattata
taaatataaagtagtaaaaattgaaccattaggagtagcacccaccaagg
caaagagaagagtggtgcagagagaaaaaagagcagtgggaataggagct
ttgttccttgggttcttgggagcagcaggaagcactatgggcgcagcgtc
aatgacgctgacggtacaggccagacaattattgtctggtatagtgcagc
agcagaacaatttgctgagggctattgaggcgcaacagcatctgttgcaa
ctcacagtctggggcatcaagcagctccaggcaagaatcctggctgtgga
aagatacctaaaggatcaacagctcctggggatttggggttgctctggaa
aactcatttgcaccactgctgtgccttggaatgctagttggagtaataaa
tctctggaacagatttggaatcacacgacctggatggagtgggacagaga
aattaacaattacacaagcttaatacactccttaattgaagaatcgcaaa
accagcaagaaaagaatgaacaagaattattggaattagataaatgggca
agtttgtggaattggtttaacataacaaattggctgtggtatataaaatt
attcataatgatagtaggaggcttggtaggtttaagaatagtttttgctg
tactactatagtgaatagagttaggcagggatattcaccattatcgtaca
gacccacctcccaaccccgaggggacccgacaggcccgaaggaatagaag
aagaaggtggagagagagacagagacagatccattcgattagtgaacgga
tctcgacggtatcggttaacttttaaaagaaaaggggggattggggggta
cagtgcaggggaaagaatagtagacataatagcaacagacatacaaacta
aagaattacaaaaacaaattacaaaaattcaaaattttatcg

Other plasmids used in projects in the patent and the interesting sequences:

Firefly Luciferase-Red fluorescent protein (Fluc-RFP) fused protein: pcDNA3.1(+)/Luc2=tdT, Addgene 43904

Humanized Gaussia luciferase (hGluc): pSV40Gluc Plasmid, New England BioLabs, Inc. Cat #: N0323S.

Matrix Metallopeptidase 1 (MMP1): MMP1 (NM_002421) Human cDNA ORF Clone, Origene Technology, Inc Cat #: RG202460

Beta galactosidase: pOPINVL, Addgene 26040

### Example 2: A Stem Cell Approach for Cancer Blood Test

### Introduction

Cancer is a leading cause of human morbidity and mortality, and its origins, biomarkers and detection remain difficult to pinpoint^{[5]}. While early detection has proven to be a useful and often necessary first step to effectively manage and treat cancer^{[27]}, it remains a challenge to identify cancer at early-stages, especially small tumors and metastases which account for over 90% of cancer mortality^{[6, 28]}. Methods of cancer detection based on imaging are non-invasive, but common drawbacks include high cost, low specificity or resolution, and the use of potential irritating contrast agents^{[27]}. For instance, positron emission tomography (PET), computed tomography (CT), and their combinations (PET-CT), are widely used for identifying and staging tumors, but require high doses of ionizing radiation and have limited specificity and resolution^{[29]}. Other imaging modalities, such as magnetic resonance imaging (MRI) and ultrasound, do not use radiation but are still unable to achieve spatial resolution smaller than several millimeters^{[30, 31]}. On the other hand, tissue biopsies are invasive and suffer from false negatives for heterogeneous tumors, and obtaining biopsies from multiple small disseminated tumors (e.g., metastases) is impractical. Cancer screening also utilizes tests for biomarkers, including circulating tumor cells, exosomes, proteins and nucleic acids. Recently, scientists have developed nanoparticle-based synthetic biomarkers composed of mass-encoded peptides that can be released upon tumor protease cleavage, and then detected in urine^{[32, 33]}. Such approaches, however, still rely on passive delivery of nanoparticles to tumor via the enhanced permeability and retention (EPR) effect and on limited types of endogenous proteins, both of which are cancer type-specific. Nevertheless, cancer biomarker discovery has led to only a few biomarkers used in clinical diagnosis since cancer biomarkers frequently suffer from low sensitivity and specificity^{[34]}.

In particular, cancer heterogeneity and evolution makes it challenging to rely on molecular biomarkers for cancer detection^{[5]}. For example, the commonly used cancer biomarkers prostate specific antigen (PSA) for prostate cancer and BRCA1/2 gene mutations for breast cancer can only identify about 25% and 10 to 25% of the patients in each cancer type, respectively^{[35-37]}. Indeed, it has been widely accepted that a single biomarker typically lacks the sensitivity and specificity that is necessary for useful diagnosis. Intriguingly, recent research indicates that most cancers are caused by stochastic events rather than predictable mutations^{[38]}. Thus, finding biomarkers that recognize multiple types of cancers with no common genetic basis is likely less promising than previously thought. In summary, there is clearly an unmet clinical need for sensitive early-stage cancer and metastasis tests that can “universally” identify many types of cancers independently of specific biomarkers from healthy controls and other conditions that share similar symptoms (e.g., inflammation), as well as to discriminate different (sub)types of cancers at different stages.

Cells, including immune and stem cells, act as autonomous and adaptive agents and these properties have recently been used for cancer treatment and drug delivery^{[15, 39-41]}. In particular, mesenchymal stem (or stromal) cells (MSC) have been tested as therapeutic agents due to their intrinsic regenerative and immunomodulatory features^{[16, 19, 42-45]}. MSC are under investigation for treating a wide array of diseases including diabetes, myocardial infarction, stroke and autoimmune diseases^{[2, 46, 47]}. MSC are also the world's first manufactured stem cell product to receive clinical approval (i.e., Prochymal® manufactured by Osiris was approved in Canada to treat graft-versus-host disease (GvHD))^{[47]}, suggesting they may be a safe source for diagnostic and therapeutic uses in humans. Importantly, systemically-infused MSC preferentially home to and integrate with tumors, including both primary tumors and metastases in different anatomical locations^{[2]}. As we have recently reviewed^{[16]}, mounting evidence now suggests that MSC possess leukocyte-like, active homing mechanisms for tumor tropism involving a variety of adhesion molecules (e.g., P-selectin and VCAM-1) and tumor-derived cytokines, chemokines, and growth factors (e.g., CXCL12 and PDGF). This selective and active homing ability makes MSC appealing vectors for localized delivery of therapeutics to treat cancers including gliomas, melanomas, breast cancer and lung metastases in ongoing clinical trials^{[2, 39]}. In addition, MSC engineered with probes (such as luciferase) have been used to detect and image tumors in situ^{[43, 48]}. However, imaging methods such as PET/SPECT and MRI, which are currently used for cell tracking after injection are limited by the same aforementioned disadvantages of cancer detection^{[43]}.

Exogenous MSC can be used as the basis for a simple cancer blood test (FIG. 7). provided are MSC engineered with a secreted reporter that can actively and specifically home to tumor sites regardless of the type and location of the tumors, and persist there longer compared to MSC in healthy microenvironments. MSC engineered to express humanized Gaussia luciferase (hGluc)^{[49-52]} were systemically administered to mice harboring breast cancer cells, exhibited tumor tropism and persistence, and secreted hGluc into the bloodstream of tumor-bearing mice. Thus, provided are MSC engineered with secreted reporters for use as a blood test for broad cancer screening and monitoring.

### Materials and Methods

### Cell Lines and Cell Culture

Human bone marrow MSC were obtained from the Texas A&M Health Science Center and were expanded to within passages 3-6. The cells were routinely maintained in Minimum Essential Medium α (MEM α, Life Technologies) supplemented with 15% fetal bovine serum (FBS, Atlanta Biologicals, GA) and 1% Penicillin-Streptomycin (PenStrep, 100 U/ml, Life Technologies) at 37° C. in a humidified incubator containing 5% CO₂. The human breast cancer cell line MDA-MB-231 was obtained from American Type Culture Collection (ATCC, VA). These cells were grown in Leibovitz's L-15 medium containing L-glutamine (Coming, NY), and supplemented with 10% FBS and 1 U/ml PenStrep at 37° C. in a humidified incubator without CO₂. The 293T-LV cell line (Gen Target, CA) was cultured in Dulbecco's Modified Eagle Medium (DMEM, Life Technologies) supplemented with 15% FBS, Non-Essential Amino Acid (NEAA, 1×, 100 U/ml, Life Technologies) and 1 U/ml PenStrep at 37° C. in a humidified incubator containing 5% CO₂.

### Generation of Lentiviral Vectors

The following lentiviral vectors were used in this study: LV-eGFP, LV-Fluc-RFP and LV-hGluc. The sequences of interest from pUCBB-eGFP (Addgene #32548), pcDNA3.1(+)/Luc2=tdT (Addgene #32904) and pSV40-Gluc (New England BioLabs) were cloned into the promoterless lentiviral transfer vector LV-PL4 (GenTarget, CA).

### Lentiviral Transduction

All lentiviral constructs were packaged (pMD2.G, Addgene #12259; pRSV-Rev, Addgene #12253; pMDLg/pRRE, Addgene #12251) as lentiviral (LV) vectors in 293T-LV cells^{[53]} using Lipofectamine® LTX and PLUS™ Reagents (Life Technologies). MSC and breast cancer cells were transduced with LVs by incubating virions in a culture medium containing 100 g/ml protamine sulfate (Sigma). After selection with medium containing 10 g/ml Puromycin (MP Biomedicals, CA), cells were visualized for fluorescent protein expression using fluorescence microscopy.

### In Vitro Bioluminescence Assays

LV-Fluc-RFP MSC (Fluc-RFP-MSC) expressing firefly luciferase (Fluc), or LV-hGluc MSC (hGluc-MSC) expressing humanized Gaussia luciferase (hGluc) were seeded in serially diluted concentrations. After the cells were washed with PBS (Lonza), luciferase substrates (150 g/ml D-Luciferin for Fluc, Perkin Elmer, MA or 20 μM coelenterazine (CTZ) for hGluc, NanoLight Technologies, AZ) were added and the activities of Fluc and hGluc were then imaged as previously described^{[54]}. Conditioned medium (CM) of hGluc-MSC was harvested and filtered. 5 μl CM was then mixed with human serum (Atlanta Biologicals, GA) with or without PBS dilution to final serum concentrations of 0%, 5%, 50% or 100%, incubated at 37° C. at various times as indicated and hGluc activity was measured with 20 μM CTZ (final concentration in a final volume of 200 μl). Mouse blood was collected as described^{[55]} and added into ¼ volume of EDTA (Sigma) solution (50 mM, pH=8.0). 5 μl blood was mixed with 100 μl of 100 μM CTZ and hGluc activity was measured immediately.

All bioluminescent assays were performed with an IVIS Lumina (Caliper LifeSciences, MA) or a plate reader (BioTek, VT). All samples above were measured in triplicate.

### Cell Implantation and Imaging In Vivo

0.5×10⁶ (2.5×10⁶/ml in DPBS) LV-Fluc-RFP MDA-MB-231 (Fluc-RFP-231) or LV-eGFP MDA-MB-231 (eGFP-231) breast cancer cells were implanted intravenously (i.v.) into NOD-SCID gamma (NSG) mice (5 weeks, #005557, The Jackson Laboratory). 5 weeks later, in vivo Fluc activity from Fluc-RFP-231 cells was measured as described^{[56]}. Briefly, in vivo Fluc signal was imaged with IVIS Lumina 10 minutes after intraperitoneal (i.p.) injection of D-Luciferin (150 mg/kg in DPBS, Lonza) into mice. 1×10⁶ hGluc-MSC or Fluc-RFP-MSC (5×10⁶/ml in DPBS) were systemically infused into the mice harboring of breast cancer cells and into healthy control mice. hGluc-MSC were labeled with the Dil lipophilic dye (5 μl/10⁶ cells, Life Technologies) by incubation at 37° C. for 20 minutes before infusion. Mice were anesthetized with 2 to 3% of isoflurane (Western Medical Supply, CA) and in vivo Fluc activity was measured at the indicated time points. Imaging was performed with the IVIS Lumina (n=4 in each case). All animal experiments and procedures were performed after the approval from the University of California, Irvine (UCI) Institution of Animal Care and Use Committee (IACUC protocol number 2012-3062) and conducted according to the Animal Welfare Assurance (#A3416.01).

### Tissue Processing and Immunohistochemistry

Tissues were collected and flash frozen in Tissue-Tek® O.C.T™ Compound (Sakura Finetek, CA), with or without overnight fixation in 4% paraformaldehyde (Amresco, OH), and with overnight incubation in 30% sucrose solution (Amresco, OH). Sections 8 μm thick were taken by cryostat and stained following an immunohistochemistry protocol for eGFP (sheep polyclonal IgG, Pierce Biotechnology) and Fluc (rabbit polyclonal IgG, Abcam). Briefly, slides were fixed in acetone (Thermo Fisher Scientific) at −20° C. for 10 minutes, permeabilized in 0.1% Triton X-100 (Sigma) for 10 minutes, and blocked in 0.1% Triton X-100 with 5% normal donkey serum (Sigma) for 30 minutes. Primary antibodies were diluted 1:100 from the stock solution and applied overnight at 4° C. Slides were washed in 1×PBS, then secondary antibodies (donkey anti-sheep IgG conjugated to Alexa Fluor® 488, donkey anti-rabbit IgG conjugated to Alexa Fluor® 594, Jackson Immunoresearch, PA) were diluted 1:500 from the stock solution and applied for 30 minutes at room temperature. Slides were washed in PBS and mounted with DPX (Sigma) or Fluoromount-G (Southern Biotech, AL). DAPI (50 g/ml, Life Technologies) in PBS was added onto slides before mounting.

### Statistical Analysis

Data were analyzed by Student's t test when comparing 2 groups and by ANOVA when comparing more than 2 groups. Data were expressed as mean±SD or mean±SEM, and differences were considered significant at P<0.05.

### Results

Humanized Gaussia Luciferase is Secreted from Engineered MSC In Vitro and is Stable in Blood.

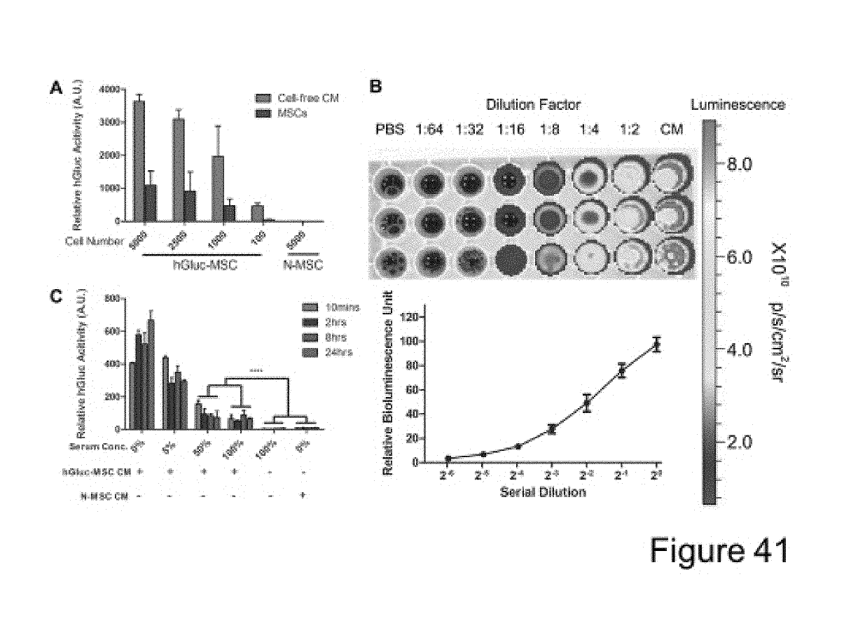

Human bone marrow mesenchymal stem cells (MSC) were stably transduced with lenti-virus to express secreted humanized Gaussia luciferase (hGluc) as described above. In order to determine whether hGluc is secreted in an active form by MSC, cell-free conditioned medium (CM) was harvested from hGluc-MSC 24 hours after MSC seeding at different concentrations (100, 1000, 2500 or 5000 cells per cm²). The substrate coelenterazine (CTZ) was added and hGluc activity was measured for both cells and CM (FIG. 41A). Measured hGluc activity increased with increasing cell number (FIG. 41A). In addition, hGluc activity in CM was 3-6 fold higher than inside cells (FIG. 41A), indicating that hGluc expressed by engineered MSC is secreted in active form, as expected. hGluc-MSC CM was serially diluted with PBS and hGluc activity was measured in vitro and found to exhibit a linear function of concentration, in agreement with earlier reports^{[54, 57, 58]} (FIG. 41B). To demonstrate whether luciferase activity from hGluc-MSC is detectable and sufficiently stable in blood, human serum either directly (100%) or serially diluted in PBS as mixed with hGluc-MSC CM. hGluc activity remained detectable (P<0.0001) after 24 hours co-incubation and hGluc activity was not decreased significantly over time (FIG. 41C), indicating that hGluc-MSC can be a stable marker in blood assays in vitro. These two luciferases were substrate-specific and no cross-reaction was observed, as reported. Overall, these data show that hGluc expressed by engineered MSC is secreted in vitro, is stable in human serum for up to 24 hours and exhibits substrate-specific enzyme activity.

Engineered MSC Home to Tumor Sites and Persist Longer than in Tumor-Bearing Mice.

As MSC are reported to naturally home to tumor sites^{[42, 43]} we then tested this phenomenon in our experiment as a preliminary step to using MSC that secrete hGluc as a diagnostic tool for cancer detection and localization. Human breast cancer-derived MDA-MB 231 cells were labeled with eGFP or Fluc-RFP and implanted intravenously (i.v.) into immunodeficient NOD-SCID gamma (NSG) mice to establish a simple in vivo mouse model of breast cancer that has metastasized in the lungs^{[59, 60]}. As hGluc is secreted by MSC, and due to its diluted and limited signal under whole animal imaging conditions with IVIS Lumina^{[61]} (data not shown), we used MSC engineered with intracellular Fluc-RFP^{[62]} for real-time imaging and localization of MSC in tumors in situ. Fluc-RFP-MSC were simultaneously labeled with red fluorescent protein (RFP) to assess Fluc transduction efficiency and to image any co-localized MSC and tumor cells in subsequent ex vivo immunohistochemistry.

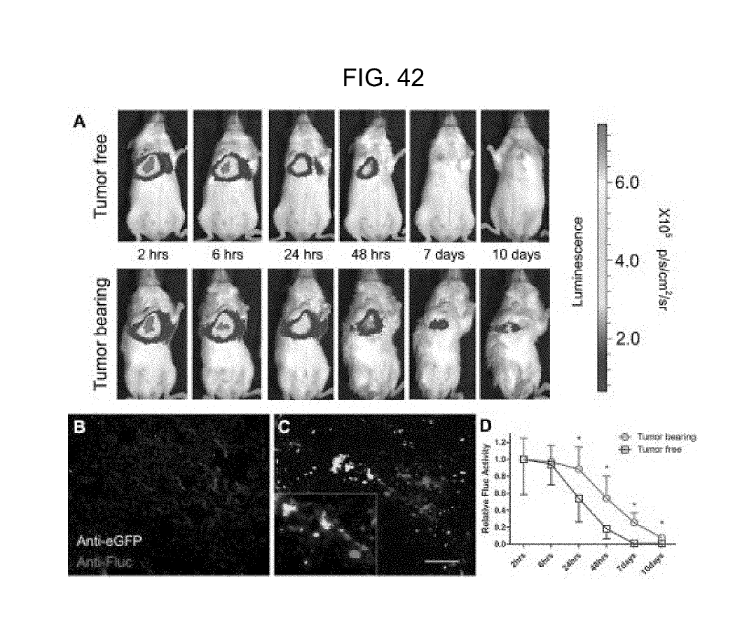

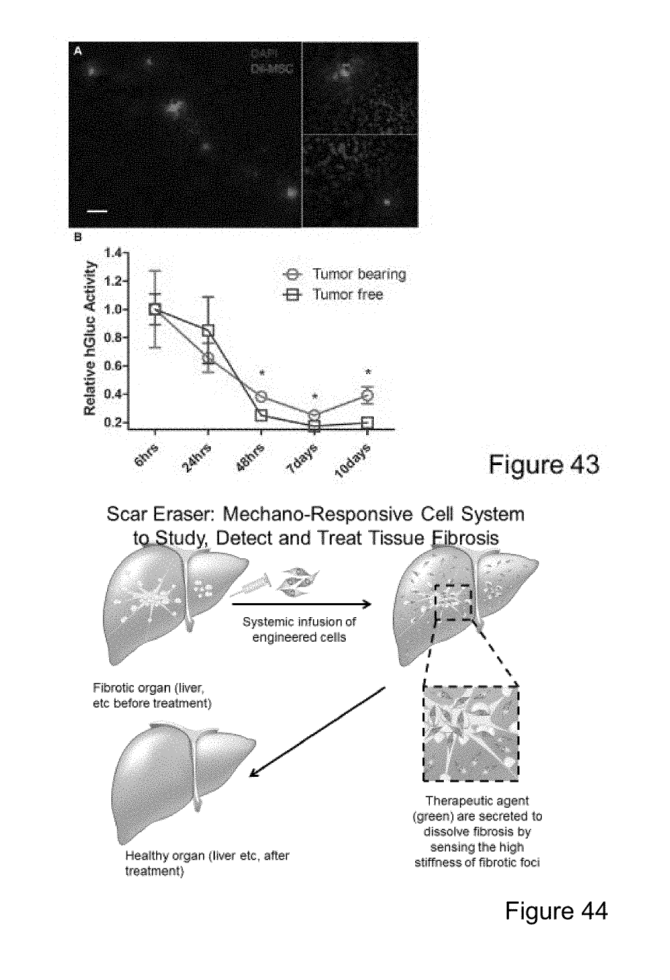

In order to investigate any differences in MSC homing between cancer-bearing and healthy mice, 1×10⁶ Fluc-RFP-MSC were systemically injected into mice with or without breast cancer. Mice were anesthetized and in vivo Fluc activity was measured after i.p. administration of D-Luciferin substrate into mice at the indicated time points. In vivo imaging demonstrated that MSC were detectable in tumor-bearing mice for as long as 10 days after systemic administration (FIG. 42A). Ex vivo immunohistochemistry data confirmed that engineered MSC homed to the tumor niche in vivo (FIGS. 42C and 43A). As we hypothesized, engineered MSC persisted significantly longer in tumor-bearing lungs, especially at later time points (FIG. 42A), consistent with several previous studies performed on different cancer models^{[16, 42]}. We then quantified the Fluc signal and found that significant differences between tumor-bearing and tumor-free mice emerged 24 hours after MSC infusion and lasted until 10 days after infusion (FIG. 42D, n=4, P<0.05). These results revealed that engineered MSC could home to and stay in tumor-bearing lungs for a significantly longer time compared to tumor-free lungs. Therefore, the in vivo persistence of engineered MSC in tumor-bearing compared to healthy animals may provide a viable “marker” for broad cancer detection.

hGluc secreted by MSC can be assayed in the blood of tumor-bearing mice.

We next investigated whether MSC that were engineered to express hGluc can be used to detect metastasis of breast cancer to the lungs. hGluc was chosen as the reporter in this study because of its high sensitivity, lack of nonspecific cross-reactivity to other substrates, and linear signal over a wide concentration range (FIG. 41B). In addition, hGluc has a short half-life in vivo (20 minutes), allowing for repeated real-time testing without undesirable excessive signal accumulation, but a long half-life in vitro (6 days), allowing for convenient sample storage^{[54]}. As hGluc is secreted, it cannot be used as a marker to co-localize MSC and tumor as seen in FIG. 42C for intracellular Fluc. Therefore, in this set of experiments, we stained hGluc-MSC with the Dil lipophilic dye before they were infused i.v. into mice. Like Fluc-RFP-MSC, Dil-MSC were detectable in the tumor niche up to 10 days post-infusion (FIG. 43A). Mouse blood was collected at the indicated time points and hGluc activity was measured. Although the detected signal decayed rapidly over time as expected, the difference of hGluc activity in blood between tumor-bearing and tumor-free mice was significant starting from 48 hours after MSC administration and lasting until 10 days post-infusion (FIG. 43B), suggesting that systemically-infused hGluc-MSC can be used for the potential development of a simple blood assay for cancer detection in this murine model. In summary, this set of data supports the feasibility of using engineered MSC with secreted hGluc as a blood test for the presence of cancer.

### Discussion

Early detection of cancer, and especially metastasis, is a necessary and often critical first step to effectively treat and eradiate cancer. Traditional imaging tools and molecular biomarker-based assays are typically complex, expensive and/or invasive for routine screening for most cancers; most importantly, they frequently do not possess the sensitivity and specificity to identity heterogeneous cancers at early-stages.

Provided are stem cell-based detection systems that can detect cancer, including metastases, by collecting small amounts of blood with a minimally invasive procedure. Our engineered MSC could home to tumor sites and persist there for significantly longer durations compared to healthy mice. The signal derived from engineered stem cells lasted longer compared to current imaging tracers^{[29]} and no repeat administration was needed. With one single administration, the presence of tumor could be monitored continuously through a prolonged period of time, making MSC a convenient tool for real-time cancer detection. Compared to acellular systems (e.g., antibodies and nanoparticles), the natural interactions between MSC and tumor involve complex adaptive sensing and responding systems that enable more efficient and specific reporting of cancer and metastases. This intrinsic biological property of tumor homing therefore potentially allows our stem cell approach to “universally” identify many cancers regardless of their origins, types and anatomical sites. In addition, stem cell-based probe delivery also circumvents many hurdles associated with passive delivery (i.e., by direct administration or polymeric nanoparticles via the EPR effect), including penetrating the endothelium and the increased pressure associated with tumors. Therefore, our simple, noninvasive stem cell-based blood test is useful for routine cancer screening, detecting small tumors and metastases, and monitoring cancer progression and recurrence during the course of treatment.

Since MSC possess not only tumor tropism but also tropism for bone marrow and sites of inflammation and injury^{[19, 44]}, it remains important to distinguish those conditions from cancer when using MSC-based methods to detect cancer. In addition, given high cancer heterogeneity, provided are systems for engineering MSC with activatable, cancer type-specific probes to increase the assay specificity. Provided are panels of tests that can effectively discriminate between cancer (sub)types and stages and distinguish between cancer and other disorders that share similar symptoms, including inflammation and injury.

MSC were chosen because they can be easily obtained from multiple adult tissues^{[63]}, including bone marrow and fat, therefore avoiding ethical concerns. MSC are also relatively easy to expand in culture, and can be readily engineered to express functional therapeutics or reporters^{[15, 19]}. Importantly, the clinically-approved Prochymal® and hundreds of other ongoing clinical trials have demonstrated that allogeneic MSC are generally safe for use in the human without harsh immunosuppressive regimens. Nonetheless, as MSC may themselves participate in cancer progression or regression,^{[16]} further considerations are required. The interactions between MSC and cancer remain incompletely understood^{[15, 16]}, with different reports indicating conflicting findings from endogenous and exogenous MSC on cancer progression^{[16, 64, 65]}. Thus, safety tests and optimizations will likely be required to better control the fate of our engineered MSC after cancer detection. To mitigate this potential issue, for example, a suicide gene^{[24]} can be engineered into our MSC-based system so that after completion of the cancer detection test, the remaining engineered MSC can be eliminated using exogenously administered drugs. Also, our system may be used as companion diagnostics combined with other treatments, for example, identifying certain patients and monitoring side effects. Finally, provided are cell-based blood assays that are a new platform for monitoring the fate and functions of transplanted cells as well as for assessing the in vivo microenvironment where they reside.

### Conclusion

Provided are simple blood tests for cancer detection using the natural tumor-homing ability of MSC to further engineer them to express a secreted reporter or marker, e.g., a luciferase, with optimal biocompatibility and kinetic parameters. Similar to our current murine studies, these “reporter MSC” could be developed to identify the presence of small tumors or metastases in humans that would otherwise be undetectable by existing imaging modalities. We hope this simple, “off the shelf” allogeneic stem cell-based diagnostic test can be used to screen, detect and monitor cancer on a routine basis.

### Example 3

### Scar Eraser: Mechano-Responsive Cell System to Study, Detect and Treat Tissue Fibrosis

Background: Current Problems with Tissue Fibrosis

Fibrosis is excessive fibrous connective tissue, usually formed in the body as a response to damage, i.e. scarring. Fibrosis can form as part of the normal healing process, where cells lay down extracellular matrix (ECM) to close wounds and then resolve the fibrosis at a later stage to replace it with new functional tissue. Conversely, pathological fibrosis can form due to many disease processes such as infection or autoimmune responses. In this case, there is no resolution of the healing process and the excess ECM remains. This scar tissue is often many times stiffer than normal tissue and nonfunctional, and may even obstruct the normal function of the surrounding tissue, potentially leading to organ failure and death^{[66]}.

Most organs in the body can be affected by pathological fibrosis. Some common conditions with complications attributed to tissue fibrosis include idiopathic lung fibrosis, heart failure, liver cirrhosis, and kidney failure after organ transplantation. Fibrosis is also a concern in the realms of medical implants and biomaterials, where the body's reaction to a foreign object may cause permanent inflammation and scarring^{[67]}.

Currently there are very few, if any, specific treatments for pathologic tissue fibrosis. Most are merely methods to prevent further damage, such as general anti-inflammatory and immunosuppressive medications. More serious fibrotic conditions such as idiopathic pulmonary fibrosis usually lead to organ failure and mortality within a few years. Thus, there is an enormous need for more targeted and effective methods to treat tissue fibrosis.

Provided are engineered cells that specifically target and treat tissue fibrosis. In particular, mesenchymal stem cells (MSC) are used for this purpose due to several intrinsic properties. MSC are immune privileged, relatively easy to acquire from multiple tissue sources, have natural homing to sites of inflammation, and can secret helpful anti-fibrotic and anti-inflammatory factors. Also, MSC present low chance of ectopic tissue formation or damage to healthy tissue.

Compared to native cells, engineered MSC can achieve more specific targeting and can express more useful factors. Specifically for tissue fibrosis, MSC can be engineered to express proteins such as matrix metalloproteinases (MMPs) which naturally break down ECM in normal tissue remodeling. In chronic cases of pathologic fibrosis, there is a lack of the natural processes that break down ECM and resolve the fibrotic healing events. Thus, MSC can be used to target and deliver therapeutic proteins in situ to dissolve excess scar tissue.

Provided are MSCs engineered to express matrix metalloproteinase-1 (MMP-1), or collagenase 1, a member of the MMP family that is known to break down interstitial collagen. Several studies have already shown the therapeutic benefits of MMP-1 for tissue fibrosis^{[68-70]}. However, most of these previous studies have focused on transgenic experiments rather than direct delivery, and thus lack specificity and targeted effects. A stiffness-sensing promoter will allow the engineered cells to selectively activate expression of MMP-1 only in contact with stiff, fibrotic tissues. This will allow in vivo specific detection and targeted treatment of tissue fibrosis (FIG. 44).

Provided are platform technologies that will allow the cells to be engineered with many other factors that have also shown promise in treating tissue fibrosis, such as hepatocyte growth factor (HGF), other members of the MMP family, TGF-β, and more.

Provided are engineered cells can specifically target and deliver therapeutic proteins to dissolve excessive tissue fibrosis, thus improving organ function.

### Experimental Design

### In Vitro Studies:

Provided are cells engineered by inserting the therapeutic gene through transfection as seen in FIG. 2. To validate the specific activation of the engineered cells, the MSC can also be engineered to express a reporter gene such as green fluorescent protein (GFP) following the stiffness-sensing promoter. MSC can then be plated on both stiff and soft substrates and imaged for the presence of the reporter. Successfully engineered cells should activate the reporter, and thus the therapeutic gene, only on stiff substrates. In vivo, this would translate to engineered MSC only secreting MMP-1 on fibrotic regions of the tissue, but not healthy unscarred tissue.

To test the functional aspect of the engineered cells, MMP secretion levels can be quantified using various assays such as ELISA. The cells can then be seeded on fluorescently labeled ECM gels to observe MMP activity as the ECM is degraded.

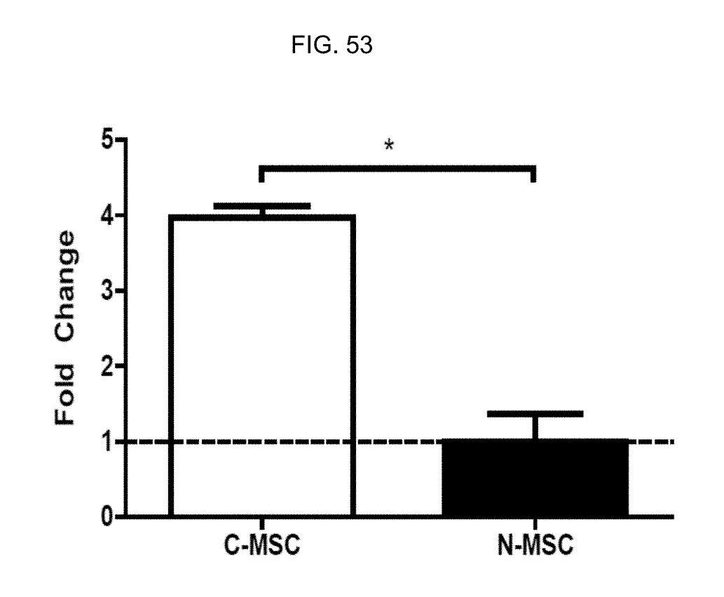

To test the protein expression levels of MMPs in engineered MSC, conditioned medium was collected from MSC engineered to overexpress and secrete MMP-1. Protein levels in the medium were quantified using ELISA. FIG. 53 shows the expression level of MMP-1 is much higher in the engineered cells as compared to native MSC. mRNA expression can be quantified using western blot and qPCR. Functional assays for the enzyme activity can be measured using a FRET-based assay specific to MMP-1.

### In Vivo Studies:

Since fibrosis can affect most organs, provided are several animal models to test the effects of engineered cells on fibrosis in vivo. Some murine models to simulate human tissue fibrosis are bleomycin-induced lung fibrosis, isoproterenol-induced global cardiac fibrosis and carbon tetracholoride (CCl₄) induced liver fibrosis.

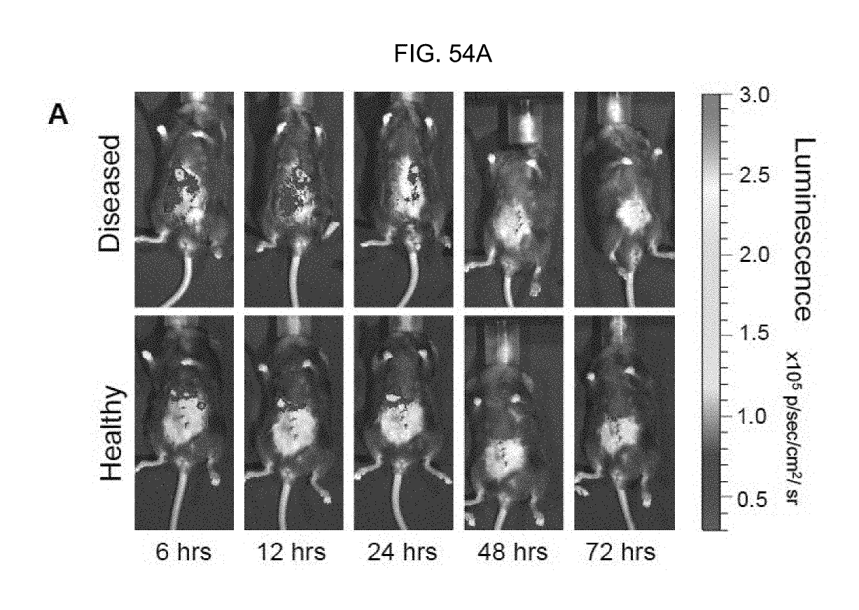

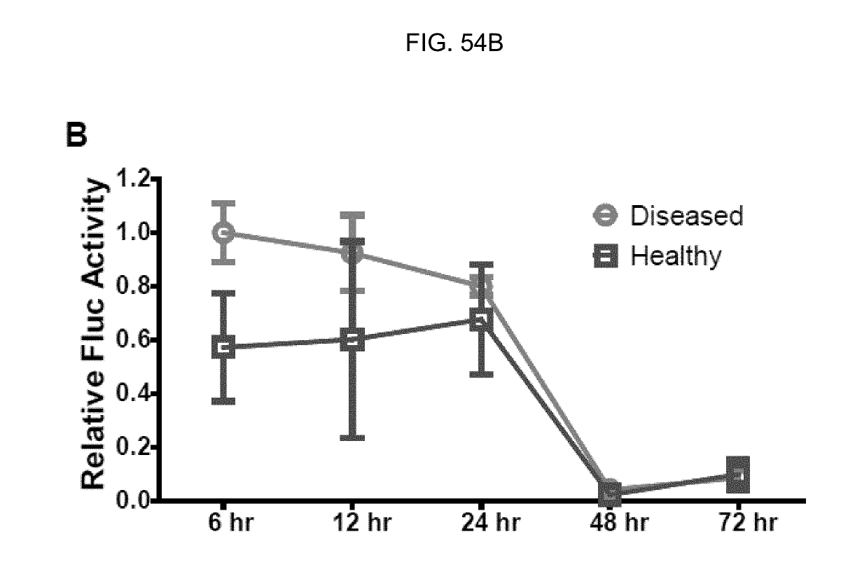

In vivo live imaging can be done with IVIS Lumina to show localization of infused MSC to organs of interest. In a murine model for liver fibrosis, mice were first injected with CCl4 to induce the formation of fibrosis for 6 weeks. Then, MSC expressing Firefly luciferase were injected via the portal veins and mice were imaged live over the next 72 hours to track the homing and retention of the cells. FIG. 54 shows the cells remain in the liver after injection for at least 2 days.

Therapeutic gene expression can be confirmed after infusion of cells in vivo via PCR. Histology can be used to quantify the extent of fibrosis via connective tissue stains such as Masson's Trichrome or picrosirius red. Immunnohistological studies can confirm the colocalization of infused MSC and fibrotic regions. Second harmonic generation (SHG) imaging can also be used to determine the localization and extent of fibrosis within the tissue. Mechanical properties of fibrotic versus healthy tissue can be characterized using atomic force microscopy (AFM).

### Example 4: Cell Tracker: A Cell Status Tracking System Using Blood Test with IC 3D

### Current Problems of Cell Transplantation

Current technology cannot accurately monitor the status of the cells in vivo in real time over a long period of time (i.e., the cell fate of MSC after transplantation), and is impossible to accurately determine the location, function, activity, presence of the cells after transplantation, thus perturbation of the system is not available. In addition, many procedures require an extended period of time to determine if the transplantation was successful (i.e., HSC transplantation). Hematopoietic stem cell transplantation (HSCT), for example, requires 28 days on average to determine the successfulness of the procedure, which diminishes the window for a possible second transplantation to rescue the failure of the first one. Therefore, there is an urgent clinical need for tracking the statues of cells after transplantation.

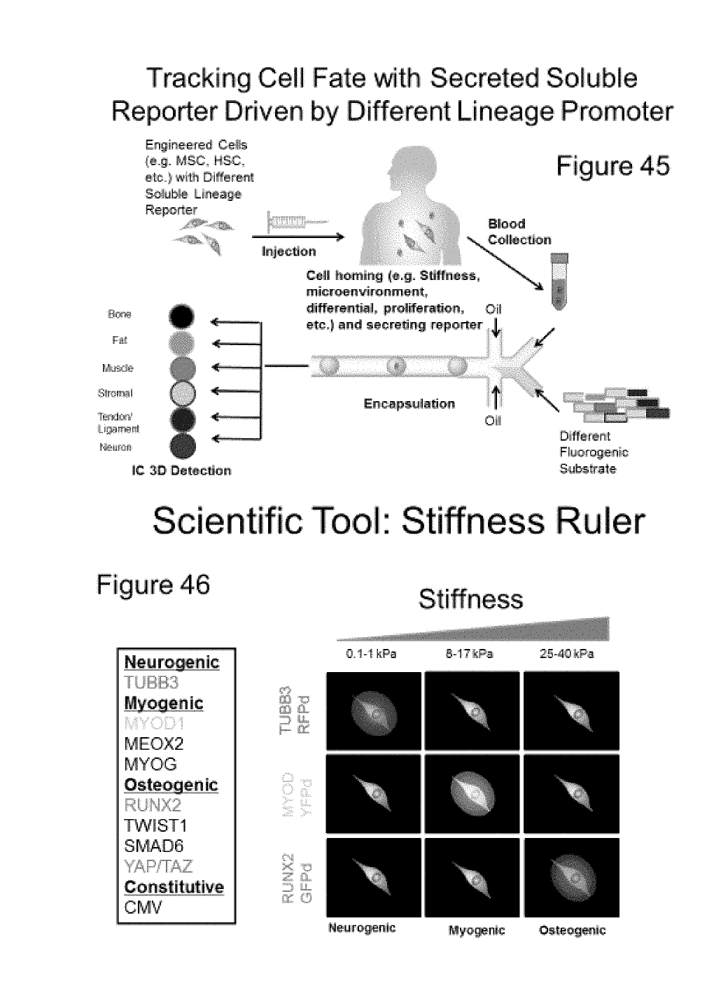

Provided are systems that can monitor the fate and function of transplanted stem cells with minimal invasiveness, ultrasensitive detection for minimum number of cells, can monitor over a long period of time, and have no detrimental effect. Exemplary embodiments are summarized in FIG. 7; for example, cells are engineered with exogenous soluble reporter enzymes after specific promoter (i.e., YAP/TAZ for stiffness sensing). After transplanting the engineered stem cells into patients, the reporter enzymes will be expressed after the cells home to specific niche (e.g., tumor niche) and secrete the enzymes into blood, which can be detected with blood test. The blood test is coupled with ultrasensitive detection methods, such as integrated comprehensive digital droplet detection (IC 3D). The blood sample is compartmentalized into picoliter-size droplets in oil, containing one or no enzyme in each droplet, and the droplets containing reporter enzymes will react with their specific fluorogenic substrate. The fluorescent droplet can be detected with 3D particle counter. Thus, the presence and functioning of the transplanted cells can be monitored in real-time with a simple blood test. In addition to cell presence, the system can further be used to track the lineage and fate of cells after transplantation (FIG. 45). Cells are engineered with different lineage-specific secreted soluble reporters. The engineered cells are infused into patient. After the cell home to the specific niche and secrete reporters into the blood, a small portion of blood is collected. The collected blood is then encapsulated with different fluorogenic substrates specific to each reporter into picoliter-sized droplets. With the presence of reporter, the droplet will become fluorescent, and the color of fluorescence reflects cell lineage. For example, stem cells are engineered with different exogenous soluble reporter enzymes after each lineage-specific promoter (e.g., bone promoter-Gluc, muscle promoter-HRP, etc.). The specific reporter enzymes will be expressed after the stem cell differentiated into the corresponding cell lineage (e.g., Gluc is expressed when the stem cell has differentiated into bone cell, etc.) and secreted out of the cells. After transplanting the engineered stem cells into the recipient, the differentiation and lineage ratio of the cells can be monitored by blood test for the secreted reporter enzyme in the blood. The blood test is coupled with ultrasensitive detection methods, such as integrated comprehensive digital droplet detection (IC 3D). The blood sample is compartmentalized into picoliter-size droplets in oil, containing one or no enzyme in each droplet, and the droplets containing reporter enzymes will react with their specific fluorogenic substrate. The fluorescent droplet can be detected with 3D particle counter.

IC 3D has previously been demonstrated with detection sensitivity of as low as one molecule per milliliter. The reagents (e.g., blood sample containing targets, and sensors for targets) are mixed in oil, generating picoliter-size water-in-oil droplets that either contains one or no target. Within the droplet containing target, the sensor and targets will react and generate fluorescent product. The fluorescent droplet can be detected with 3D particle counter to determine the number of fluorescent droplet, which corresponds to the number of target contained in the original sample. Assuming one stem cell may produce 1000 reporters, then 10 stem cells would give approximately 1 to 10 reporters per milliliter. Current technology (e.g., flow cytometry) cannot detect the presence of 10 stem cells within the body, while our soluble reporter IC 3D system is able to detect 1-10 molecules per milliliter of blood. In addition, most of stem cells (i.e. MSC, NSC) home to their niche and become immobile after transplantation, which makes them unavailable to most of current detection methods.

Provided are methods, e.g., blood tests, that use soluble reporters which in blood samples can accurately assess cell status, and this blood test is more sensitive than current methods (e.g., flow cytometry, PCR, etc.)

### Experimental Design

To accomplish the detection system, one of the key components is the exogenous enzyme to be engineered into the cells. The requirements for the reporter enzymes are that they need to be exogenic, highly active, non-pathogenic with suitable half-life. We have identified a group of the enzymes possessing the features mentioned above, including E. coli beta-galactosidase (E. coli beta-gal) and horseradish peroxidase (HRP). Both of the enzymes have been used for in vivo studies with no reports of toxicity, and have been demonstrated with single enzyme activity assay. We have characterized E. coli beta-galactosidase with cross-reactivity against human serum, and found that human serum have no cross-reactivity with E. coli beta-gal or fluorescein Di-beta-D-galactopyranoside (FDG), the substrate for E. coli beta-gal.

Next, E. coli beta-gal is cloned into transduction vector with constitutive promoter (e.g., beta-actin), which are used to engineer mouse HSC. The engineered HSC are transplanted into a recipient mouse whose bone marrow has been lethally depleted with 5-fluorouracile (5-FU). After transplantation, the E. coli beta-gal content is monitored continuously and compared with flow cytometry data to validate the platform.

After the platform is validated, multiple reporter enzymes are engineered into stem cells, each after a specific lineage promoter to study other stem cell systems (e.g., MSC, NSC, induced pluripotent stem cells (iPSC), etc.).

### Example 5: Early Detection of Hematopoietic Stem Cell Engraftment after Transplantation

### Background

Leukemia is the most common cancer in children, accounting for approximately 30% of all cases^{[71]}. The transplantation of bone marrow containing hematopoietic stem cells (HSC) from closely matched donors represents the best therapy for childhood malignancies^{[72]}. However, HSC transplantation (HSCT) can cause clinical complications. Failure of the HSC to reconstitute the immune system in the patient occurs in 4% of the cases^{[73]}.

Early prediction of graft failure allows for a timely second HSCT or other therapeutic interventions. However, current technologies such as flow cytometry require 2-4 weeks to determine HSCT success or failure^{[74]}, and may only detect post-transplantation chimerism after recovery^{[75]} without accurately monitoring the early failure of HSCT. Therefore, there is an unmet clinical need for tracking the HSC status early after transplantation and in a minimally invasive fashion. Current in situ cell imaging methods including PET/SPECT and MRI are limited by low specificity, resolution, and the use of potential irritating contrast agents^{[76]}. Indeed, the inability to monitor and manipulate the fate of transplanted cells in human remains a biggest bottleneck of successful cell transplantation.

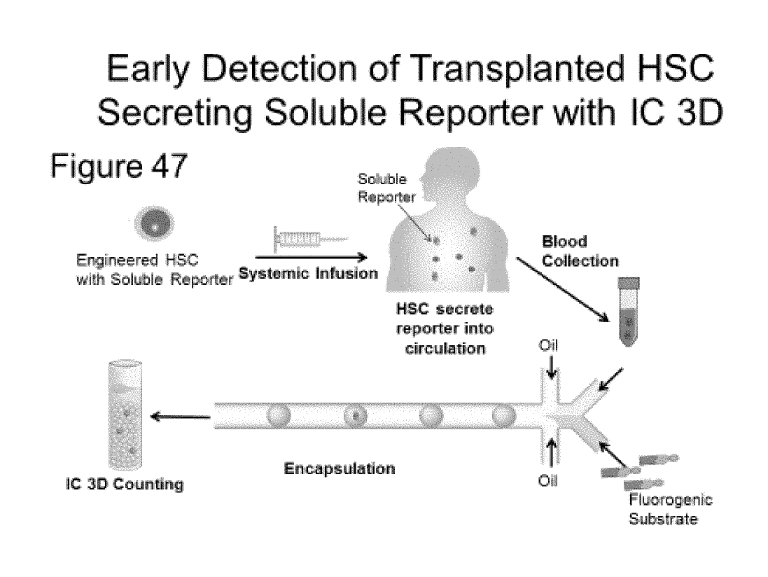

Provided are ultrasensitive detection platforms, e.g., so-called Integrated Comprehensive Digital Droplet Detection (IC 3D), able to detect target molecules or cells in blood with single-molecule or single-cell sensitivity^{[77]}. HSC can be used to track HSCT which combines HSC lineage tracing with our IC 3D (FIG. 47); Specifically, HSC can be engineered to constitutively express secretory beta-galactosidase (beta-gal) as a soluble reporter for HSC, and the blood beta-gal level correlates with HSC number. HSC will also be engineered with soluble reporters following lineage-specific promoters (e.g. CD3 promoter-beta-gal/FDG pair for T-Cell lineage, Ig-E promoter-HRP/QuantaBlu pair for B-Cell lineage). The soluble reporter level in blood can be determined with the abovementioned IC 3D system. Since massive reporter enzymes can be secreted by one cell, it is anticipated that HSC engraftment and lineage reconstitution can be detected much earlier with the soluble reporter than the current standard flow cytometry.

The functions of transplanted cells, even at single-cell level in vivo, can be monitored longitudinally using exemplary ultrasensitive blood assays that measure secreted probes that are coded for a particular cell function.

### Experimental Design

### Detect Soluble Reporter in Blood Using IC 3D

IC 3D can detect soluble reporter in blood at single molecule level and characterize the reporters (enzymes and their substrates) in vitro. E. coli beta-galactosidase (beta-gal) will be used because it has been previously demonstrated with single-enzyme detection in vitro^{[78]} and in vivo^{[79]} with a plasma half-life is less than 60 min^{[80]}, and may last 5 hours after blood collection^{[81]}.

Provided are 1) recombinant enzyme assays with conventional methods as well as IC 3D; The IC 3D encapsulates reporter enzyme-detecting fluorescent sensors into picoliter-size droplets, and the reporter enzyme-containing droplets are detected with a high throughput droplet counting system. Our data have demonstrated that single beta-gal can be encapsulated and visualized with fluorescent microscope. Droplet size and reaction condition can be optimized to ensure single-enzyme sensitivity with IC 3D.

Experiment 2: Engineer and Characterize HSC with Secreted Probes

Genetically engineer HSC with reporter enzymes downstream of constitutive promoters (e.g. beta-actin) and lineage-specific promoters (e.g. CD3 promoter-beta-gal/FDG pair for T-Cell lineage, Ig-E promoter-HRP/QuantaBlu pair for B-Cell lineage). We will use the lenti-viral transfer vectors that have been safely used in clinic trials^{[82, 83]}. The reporter enzyme expression will be characterized with IC 3D.

### Experiment 3: Testing the Lineage-Tracing/IC 3D Approach for HSCT Characterization In Vivo

Congenic mouse HSCT model made using following previous work^{[84]}: methods comprise 1) lethal bone marrow depletion of CD45.2 mice with 5-fluorouracil (5-FU) and irradiation, 2) transplantation of the engineered CD45.1 HSC into depleted CD45.2 mice^{[85, 86]}.

After host bone marrow ablation and HSCT, collect peripheral blood to monitor: 1) blood-beta-gal level via IC 3D, and 2) blood-CD45.1-cell type content via flow cytometry at 24 H, 72 H, 7 D, 14 D, 21 D and 28 D post-transplantation. These experiments allow us to detect various reconstituted donor cells, including neutrophils and T and B lymphocytes to validate the success of the model. Correlation of blood-beta-gal level to HSC cell counts will also allow us to determine whether the transplantation is successful, thus providing clinically useful information.

### Example 6: Stiffness Ruler In Vivo Stiffness Detection: Mechano-Sensing Cells for Measuring Tissue Stiffness in Native Cellular Environment

Background: The Need for In Vivo Stiffness Detection

Mechanobiology is an emerging field of study that focuses on the effects of physical cues, such as stiffness, on cell and tissue physiology. Whereas previous physiological research was primarily focused on biochemical pathways, it is now acknowledged that mechanical properties of tissues are also central to development, function, and disease states. Abnormal tissue stiffness is a hallmark of many pathologic states such as fibrosis, inflammation, and cancer. For example, carcinomas can have 10-fold higher elastic modulus compared to healthy tissue^{[87]}. The ability to detect tissue stiffness in vivo in the native cellular environment can be a powerful tool to study mechanobiology in the context of physiological and pathological conditions. This knowledge will have broad implications to develop future diagnostics and therapeutics that directly target the biophysical cues as novel biomarkers.

However, a major challenge in this field is to study mechanobiology in vivo, specifically, the interplay of biomechanical cues with cells in their native environment. Current methods of tissue mechanical studies fall into two general categories: imaging and mechanical testing. Imaging modalities, including especially elastography, suffer from poor sensitivity and resolution and are not able to study mechanobiology at the cellular level in a high spatiotemporal resolution. Ex vivo mechanical testing of tissues using atomic force microscopy (AFM) indentation and microrheology require invasive biopsies and do not replicate the native biological conditions.

Provided are cell-based platforms for sensitive detection of tissue stiffness in the native microenvironment. Through mechanotransduction, cells can convert mechanical cues in their surroundings to detectable biochemical signals. For example, it has been established that tissue and matrix stiffness alone can drive differential gene expression in mesenchymal stem cells (MSC) and other commonly used model cancer cell lines (including MDA-MB-231, MCF-7). Soft matrices, for instance, direct MSC to a neurogenic lineage, with expression of characteristic promoters and transcription factors^{[88]}. This endogenous ability of MSC to activate different genetic pathways can be used to drive expression of stiffness-responsive reporters or therapeutics. The ability of cancer cell lines to differentially express stiffness-responsive reporters can be used to study the mechanobiology of primary and metastatic cancer models.

Cells can be engineered with many different promoters which selectively activate on substrates within a certain range of stiffness. Promoters of listed genes responsive to specific ranges of stiffness will be cloned from genomic DNA and subcloned into promoterless vectors to drive expression of florescent proteins. Then the constructs can be permanently transduced into cells such as mesenchymal stem cells (MSC) to produce stable engineered MSC cell lines. Each of our reporters can only be turned on in the presence of the appropriate mechano-environment (FIG. 46). For example, cells engineered with a neurogenic promoter TUBB3 followed by a RFPd reporter gene will activate and express RFPd only on soft (0.1-1 kPa) substrates.

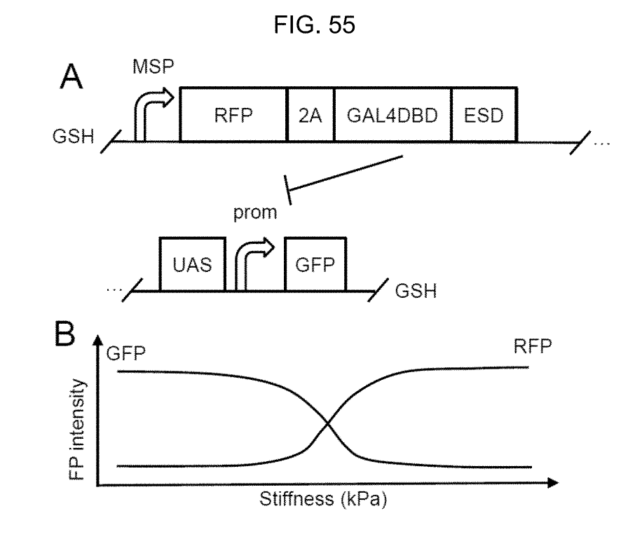

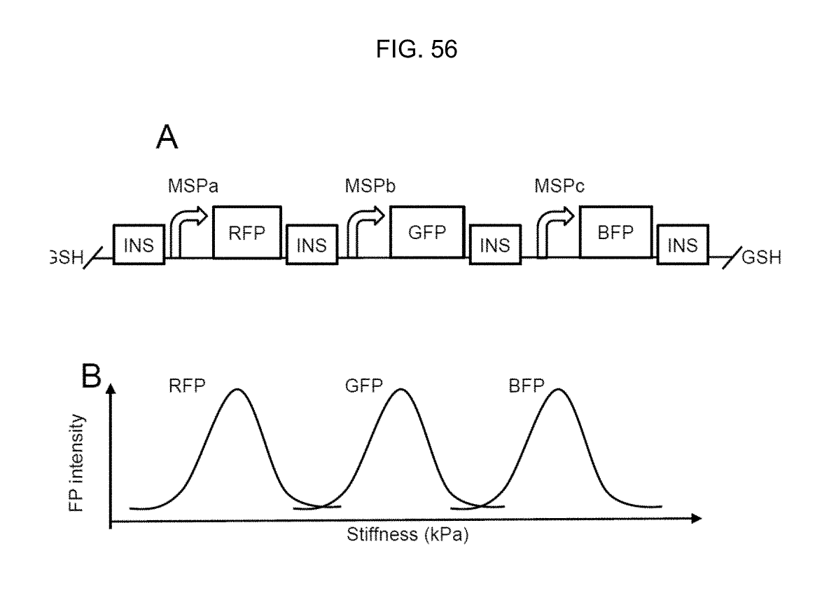

Alternatively, other more complex engineered genetic circuits are provided, they can be constructed as described e.g., in FIGS. 55 and 56, and detailed below.

Provided are “stiffness ruler” tools, including engineered cells, and methods of using them, where exemplary tools comprise a mixture of cells with different engineered promoters that selectively activate on different substrate stiffness, the selective activation on the different substrate stiffness forms a “stiffness ruler” tool. The cells will be able to locally detect and report stiffness of tissues in vivo, and provide further insight into the cell microenvironments within the body.

Provided are engineered MSC that can be used as a novel tool for studying tissue mechanobiology in vivo by expression of specific reporter genes in response to differential substrate stiffness. Provided are cell-based stiffness sensors that reveal what cells actually “feel” in their native environment and represent a paradigm-shifting method of dynamically interrogating the mechano-environment of matrix stiffness during natural biological processes, disease progression and response to therapies at the cellular resolution in vivo.

### Experimental Design

Engineer MSC with Stiffness-Responsive Promoters:

Several stiffness sensing promoters have already been identified: TUBB3 (β3-tubulin, neurogenic), MYOD1 (MyoD, myogenic) and RUNX2 (RunX2 or CBFα1, osteogenic)^{[2]}. The promoters are isolated from human genomic DNA and then cloned into a reporter construct containing sequences for destabilized fluorescent proteins with different colors (e.g., RFPd, GFPd, BFPd). Human bone marrow MSC are transduced via nucleofection. Thus far, we have successfully isolated the promoters of interest.

Alternatively, more complex engineered genetic circuits can be constructed to more finely elucidate mechano-responsive properties in vivo. First a two-state stiffness ruler can be used to more precisely screen the various promoters listed above. The two-state model functions by reporting both the ON state and OFF state of the synthetic promoter can be studied. In the ON state RFP is expressed signifying the functional expression of the mechano-sensitive promoter, while in the OFF state GFP is expressed signifying the functionally repression of the mechano-sensitive promoter. For instance, as in described various mechano-sensitive synthetic promoters (MSP) are cloned upstream of a reporter (e.g. RFP) (FIG. 55). These synthetic promoters are constructed using the known promoter regions of various genes described above that are fused to the minimal chicken TNT promoter described below. Regions of native promoters are selected based on their proximity to the transcriptional start site. Thus the expression-level of the RFP is driven by the mechano-sensitivity of the synthetic promoter. Downstream of the RFP another fragment is co-expressed with the RFP but post-translationally self-proteolytically-cleaved using a 2A peptide sequence^{[89]}. As another example, the GAL4DBD-ESD fragment is a fusion protein of the Gal4 DNA binding domain (GAL4DBD) and an epigenetic silencing domain (ESD) (FIG. 55). The ESD denotes various ESD fragments that can be used such as HDAC or KRAB to epigenetically and reversibly silence the expression of targeted promoters as has been previously described^{[90]}. This fragment binds to the Upstream Activation Sequence (UAS) that is not natively present in mammalian cells (as it comes from yeast) and is therefore orthogonal to mammalian cells. The details of the Gal4-UAS have been described extensively previously^{[89, 91]}. When the UAS sequence is adjacent to a typical mammalian promoter such as EF1alpha or CMV amongst other the downstream GFP is constitutively expressed, thus reporting the OFF state of the mechano-sensitive circuit. When the Gal4DBD-ESD fragment binds to the UAS sequence ESD fragment epigenetically modifying the promoter sequence thus silencing GFP expression. In combination with the concomitantly expressing RFP this secondary state of the genetic circuits reports the ON state. We see an example Fluorescent Protein intensity (FP intensity) output (FIG. 55B). In this embodiment at low stiffness value (in this example below 10 kPa) only GFP is being expressed. At medium stiffness values (approximately 10 kPa) both GFP and RFP are expressed. While at high stiffness values only RFP is expressed.

Next, a multi-state stiffness ruler genetic circuit is also described. We see a series of mechano-sensitive synthetic promoters outlined (MSPa, MSPb, MSPc) which are sensitive to low, medium, and high stiffness (FIG. 56A). These are promoters that are first screened in the two-state circuit described above. Then individual expression units (promoter-fluorescent reporter-poly-adenylation signal) are cloned into large vector using a modified version of a modular assembly method described previously as an example^{[92]}. These expression units are separated by distinct chromatin insulators denoted (INS in FIG. 56A) to insulate the expression of one mechano-sensitive promoter from the effects of adjacent genes and decrease the likelihood of the construct being epigenetically silenced. Details of the insulator sequences, such as the core HS4 sequence have been described previously^{[93]}. We see an example output where each fluorescent reporter is mainly expressed in the desired stiffness range due to the sensitivity range of each synthetic promoter (FIG. 55B). In both the two-state and multi-state model these engineered genetic circuits are site-specifically integrated into genetic safe harbor locations in mammalian such as the AAVS1 locus for human cells or the mROSA26 locus for murine cells^{[94]}. The integration of these constructs is via homology-directed repair and initiated by CRISPR/Cas9 endonucleases as described previously.^{[1]}

### Validate Engineered MSC In Vitro:

To validate cell reporter gene expression, the cells are cultured on collagen-coated polyacrylamide hydrogels, with tunable stiffness determined by relative concentrations of acrylamide and bis-acrylamide. MSC with each promoter selectively activate within their respective stiffness ranges. Inhibition of upstream mechanotransduction transcription factors such as YAP and TAZ can verify that cell promoters are only responsive to matrix stiffness but not to nonspecific factors such as inflammation or hypoxia.

In alternative embodiments, stiffness sensing sequences: CACATTCCA, are used, including e.g., a Minimal chicken TnT promoter (SEQ ID NO: 13)

CACATTCCACACATTCCACTGCAAGCTTGAGACACATTCCACACAT TCCACTGCAAGCTTGGCCAGTGCCAAGTTGAGACACATTCCACACATTCC ACTGCAAGCTTGAGACACATTCCACACATTCCACTGCAAGCTTCTAGAGA TCTGCAGGTCGAGGTCGACGGTATCGATAAGCTTGGGGGTGGGCGCCGGG GGGACCTTAAAGCCTCTGCCCCCCAAGGAGCCCTTCCCAGACAGCCGCCG GCACCCACCGCTCCGTGGGAC

Transfer Plasmids:

LV-PL4-CMV::eGFP

CMV enhancer+promoter: pcDNA3.1(+)/Luc2=tdT, Addgene 43904

Infusion Primers:

(SEQ ID NO: 14) Fw:
ggtggtggatccTGTACGGGCCAGATATACGC
(SEQ ID NO: 15) Rv:
ggtggtggatccGCCAGCTTGGGTCTCCCTAT
Enhanced green fluorescent protein (eGFP): Addgene:
32548: pUCBB-eGFP
Promoterless vector (GenTarget, Inc Cat# LV-PL4)
(SEQ ID NO: 16)
GgatccTGTACGGGCCAGATATACGCGTTGACATTGATTATTGACTAGTT
ATTAATAGTAATCAATTACGGGGTCATTAGTTCATAGCCCATATATGGAG
TTCCGCGTTACATAACTTACGGTAAATGGCCCGCCTGGCTGACCGCCCAA
CGACCCCCGCCCATTGACGTCAATAATGACGTATGTTCCCATAGTAACGC
CAATAGGGACTTTCCATTGACGTCAATGGGTGGAGTATTTACGGTAAACT
GCCCACTTGGCAGTACATCAAGTGTATCATATGCCAAGTACGCCCCCTAT
TGACGTCAATGACGGTAAATGGCCCGCCTGGCATTATGCCCAGTACATGA
CCTTATGGGACTTTCCTACTTGGCAGTACATCTACGTATTAGTCATCGCT
ATTACCATGGTGATGCGGTTTTGGCAGTACATCAATGGGCGTGGATAGCG
GTTTGACTCACGGGGATTTCCAAGTCTCCACCCCATTGACGTCAATGGGA
GTTTGTTTTGGCACCAAAATCAACGGGACTTTCCAAAATGTCGTAACAAC
TCCGCCCCATTGACGCAAATGGGCGGTAGGCGTGTACGGTGGGAGGTCTA
TATAAGCAGAGCTCTCTGGCTAACTAGAGAACCCACTGCTTACTGGCTTA
TCGAAATTAATACGACTCACTATAGGGAGACCCAAGCTGGCgGATCCATA
TGGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGG
CGAGGGCGATGCCACCTACGGCAAGCTGACCCTGAAGTTCATCTGCACCA
CCGGCAAGCTGCCCGTGCCCTGGCCCACCCTCGTGACCACCCTGACCTAC
GGCGTGCAGTGCTTCAGCCGCTACCCCGACCACATGAAGCAGCACGACTT
CTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGC
GACACCCTGGTGAACCGCATCGAGCTGAAGGGCATCGACTTCAAGGAGGA
CGGCAACATCCTGGGGCACAAGCTGGAGTACAACTACAACAGCCACAACG
TCTATATCATGGCCGACAAGCAGAAGAACGGCATCAAGGTGAACTTCAAG
ATCCGCCACAACATCGAGGACGGCAGCGTGCAGCTCGCCGACCACTACCA
GCAGAACACCCCCATCGGCGACGGCCCCGTGCTGCTGCCCGACAACCACT
ACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCAT
GGACGAGCTGTACAAGTAAATGCATCCATGGCGGCCGCCTCGAGCATCAC
CATCACCATCACTGATTCTCCTTACGCATCTGTGCGGTATTTCACACCGC
ATGTACTAGTGTCGACGCTAGCTCTAGATGTACAAAGTGGTGCTAGCACT
CTCAGTACAATCTGCTCTGATGCCGCATAGTTAAGCCAGTATCTGCTCCC
TGCTTGTGTGTTGGAGGTCGCTGAGTAGTGCGCGAGCAAAATTTAAGCTA
CAACAAGGCAAGGCTTGACCGACAATTGCATGAAGAATCTGCTTAGGGTT
AGGCGTTTTGCGCTGCTTCGCGATGTACGGGCCAGATATACGCGTATCTG
AGGGGACTAGGGTGTGTTTAGGCGAAAAGCGGGGCTTCGGTTGTACGCGG
TTAGGAGTCCCCTCAGGATATAGTAGTTTCGCTTTTGCATAGGGAGGGGG
AAATGTAGTCTTATGCAATACTCTTGTAGTCTTGCAACATGGTAACGATG
AGTTAGCAACATGCCTTACAAGGAGAGAAAAAGCACCGTGCATGCCGATT
GGTGGAAGTAAGGTGGTACGATCGTGCCTTATTAGGAAGGCAACAGACGG
GTCTGACATGGATTGGACGAACCACTGAATTCCGCATTGCAGAGATATTG
TATTTAAGTGCCTAGCTCGATACAATAAACGCCATTTGACCATTCACCAC
ATTGGTGTGCACCTCCAAAGCGCTCACCATGACCGAGTACAAGCCCACGG
TGCGCCTCGCCACCCGCGACGACGTCCCCAGGGCCGTACGCACCCTCGCC
GCCGCGTTCGCCGACTACCCCGCCACGCGCCACACCGTCGATCCGGACCG
CCACATCGAGCGGGTCACCGAGCTGCAAGAACTCTTCCTCACGCGCGTCG
GGCTCGACATCGGCAAGGTGTGGGTCGCGGACGACGGCGCCGCGGTGGCG
GTCTGGACCACGCCGGAGAGCGTCGAAGCGGGGGCGGTGTTCGCCGAGAT
CGGCCCGCGCATGGccgagttgagcggttcccggctggccgcgcagcaac
agatggaaggcctcctggcgccgcaccggcccaaggagcccgcgtggttc
ctggccaccgtcggcgtctcgcccgaccaccagggcaagggtctgggcag
cgccgtcgtgctccccggagtggaggcggccgagcgcgccggggtgcccg
ccttcctggagacctccgcgccccgcaacctccccttctacgagcggctc
ggcttcaccgtcaccgccgacgtcgaggtgcccgaaggaccgcgcacctg
gtgcatgacccgcaagcccggtgcctgaactagttaggtttaaacacgcg
taccggttagtaatgatcgacaatcaacctctggattacaaaatttgtga
aagattgactggtattcttaactatgttgctccttttacgctatgtggat
acgctgctttaatgcctttgtatcatgctattgcttcccgtatggctttc
attttctcctccttgtataaatcctggttgctgtctctttatgaggagtt
gtggcccgttgtcaggcaacgtggcgtggtgtgcactgtgtttgctgacg
caacccccactggttggggcattgccaccacctgtcagctcctttccggg
actttcgctttccccctccctattgccacggcggaactcatcgccgcctg
ccttgcccgctgctggacaggggctcggctgttgggcactgacaattccg
tggtgttgtcggggaagctgacgtcctttccatggctgctcgcctgtgtt
gccacctggattctgcgcgggacgtccttctgctacgtcccttcggccct
caatccagcggaccttccttcccgcggcctgctgccggctctgcggcctc
ttccgcgtcttcgccttcgccctcagacgagtcggatctccctttgggcc
gcctccccgcctggcgatggtacctttaagaccaatgacttacaaggcag
ctgtagatcttagccactattaaaagaaaaggggggactggaagggctaa
ttcactcccaacgaagaaagatctgctattgcttgtactgggtctctctg
gttagaccagatctgagcctgggagctctctggctaactagggaacccac
tgcttaagcctcaataaagcttgccttgagtgcttcaagtagtgtgtgcc
cgtctgttgtgtgactctggtaactagagatccctcagacccttttagtc
agtgtggaaaatctctagcagtagtagttcatgtcatcttattattcagt
atttataacttgcaaagaaatgaatatcagagagtgagaggaacttgttt
attgcagcttataatggttacaaataaagcaatagcatcacaaatttcac
aaataaagcattatttcactgcattctagagtggtagtccaaactcatca
atgtatcttatcatgtctggctctagctatcccgcccctaactccgccca
tcccgcccctaactccgcccagttccgcccattctccgccccatggctga
ctaattatatatttatgcagaggccgaggccgcctcggcctctgagctat
tccagaagtagtgaggaggcttattggaggcctagggacgtacccaattc
gccctatagtgagtcgtattacgcgcgctcactggccgtcgttttacaac
gtcgtgactgggaaaaccctggcgttacccaacttaatcgccttgcagca
catccccctacgccagctggcgtaatagcgaagaggcccgcaccgatcgc
ccttcccaacagttgcgcagcctgaatggcgaatgggacgcgccctgtag
cggcgcattaagcgcggcgggtgtggtggttacgcgcagcgtgaccgcta
cacttgccagcgccctagcgcccgctcctttcgctttcttcccttccttt
ctcgccacgttcgccggctttccccgtcaagctctaaatcgggggctccc
tttagggaccgatttagtgctttacggcacctcgaccccaaaaaacttga
ttagggtgatggttcacgtagtgggccatcgccctgatagacggtttttc
gccctttgacgttggagtccacgttctttaatagtggactcttgttccaa
actggaacaacactcaaccctatctcggtctattcttagatttataaggg
attagccgatttcggcctattggttaaaaaatgagctgatttaacaaaaa
tttaacgcgaattttaacaaaatattaacgcttacaatttaggtggcact
tacggggaaatgtgcgcggaacccctatagtttattatctaaatacattc
aaatatgtatccgctcatgagacaataaccctgataaatgcttcaataat
attgaaaaaggaagagtatgagtattcaacataccgtgtcgcccttattc
ccttattgcggcattagccttcctgtattgctcacccagaaacgctggtg
aaagtaaaagatgctgaagatcagttgggtgcacgagtgggttacatcga
actggatctcaacagcggtaagatccttgagagttttcgccccgaagaac
gttttccaatgatgagcacttttaaagttctgctatgtggcgcggtatta
tcccgtattgacgccgggcaagagcaactcggtcgccgcatacactattc
tcagaagacttggttgagtactcaccagtcacagaaaagcatcttacgga
tggcatgacagtaagagaattatgcagtgctgccataaccatgagtgata
acactgcggccaacttacttctgacaacgatcggaggaccgaaggagcta
accgcttattgcacaacatgggggatcatgtaactcgccttgatcgttgg
gaaccggagctgaatgaagccataccaaacgacgagcgtgacaccacgat
gcctgtagcaatggcaacaacgttgcgcaaactattaactggcgaactac
ttactctagcttcccggcaacaattaatagactggatggaggcggataaa
gttgcaggaccacttctgcgctcggcccttccggctggctggtttattgc
tgataaatctggagccggtgagcgtgggtctcgcggtatcattgcagcac
tggggccagatggtaagccctcccgtatcgtagttatctacacgacgggg
agtcaggcaactatggatgaacgaaatagacagatcgctgagataggtgc
ctcactgattaagcattggtaactgtcagaccaagtttactcatatatac
tttagattgatttaaaacttcattataatttaaaaggatctaggtgaaga
tcctattgataatctcatgaccaaaatcccttaacgtgagttacgaccac
tgagcgtcagaccccgtagaaaagatcaaaggatcttcttgagatcctat
tactgcgcgtaatctgctgcttgcaaacaaaaaaaccaccgctaccagcg
gtggtagtagccggatcaagagctaccaactctattccgaaggtaactgg
cttcagcagagcgcagataccaaatactgttcttctagtgtagccgtagt
taggccaccacttcaagaactctgtagcaccgcctacatacctcgctctg
ctaatcctgttaccagtggctgctgccagtggcgataagtcgtgtcttac
cgggttggactcaagacgatagttaccggataaggcgcagcggtcgggct
gaacggggggttcgtgcacacagcccagcttggagcgaacgacctacacc
gaactgagatacctacagcgtgagctatgagaaagcgccacgcttcccga
agggagaaaggcggacaggtatccggtaagcggcagggtcggaacaggag
agcgcacgagggagcttccagggggaaacgcctggtatctttatagtcct
gtcgggtacgccacctctgacttgagcgtcgattatgtgatgctcgtcag
gggggcggagcctatggaaaaacgccagcaacgcggcctattacggacct
ggccttagctggccttagctcacatgttctacctgcgttatcccctgatt
ctgtggataaccgtattaccgcctttgagtgagctgataccgctcgccgc
agccgaacgaccgagcgcagcgagtcagtgagcgaggaagctggaagggc
taattcactcccaaagaagacaagatatccttgatctgtggatctaccac
acacaaggctacttccctgattagcagaactacacaccagggccaggggt
cagatatccactgacctttggatggtgctacaagctagtaccagttgagc
cagataaggtagaagaggccaataaaggagagaacaccagcttgttacac
cctgtgagcctgcatgggatggatgacccggagagagaagtgttagagtg
gaggtttgacagccgcctagcatttcatcacgtggcccgagagctgcatc
cggagtacttcaagaactgctgatatcgagcttgctacaagggactttcc
gctggggactttccagggaggcgtggcctgggcgggactggggagtggcg
agccctcagatcctgcatataagcagctgctattgcctgtactgggtctc
tctggttagaccagatctgagcctgggagctctctggctaactagggaac
ccactgcttaagcctcaataaagcttgccttgagtgcttcaagtagtgtg
tgcccgtctgagtgtgactctggtaactagagatccctcagaccatttag
tcagtgtggaaaatctctagcagtggcgcccgaacagggacttgaaagcg
aaagggaaaccagaggagctctctcgacgcaggactcggcttgctgaagc
gcgcacggcaagaggcgaggggcggcgactggtgagtacgccaaaaattt
tgactagcggaggctagaaggagagagatgggtgcgagagcgtcagtatt
aagcgggggagaattagatcgcgatgggaaaaaattcggttaaggccagg
gggaaagaaaaaatataaattaaaacatatagtatgggcaagcagggagc
tagaacgattcgcagttaatcctggcctgttagaaacatcagaaggctgt
agacaaatactgggacagctacaaccatcccttcagacaggatcagaaga
acttagatcattatataatacagtagcaaccctctattgtgtgcatcaaa
ggatagagataaaagacaccaaggaagctttagacaagatagaggaagag
caaaacaaaagtaagaccaccgcacagcaagcggccgctgatcttcagac
ctggaggaggagatatgagggacaattggagaagtgaattatataaatat
aaagtagtaaaaattgaaccattaggagtagcacccaccaaggcaaagag
aagagtggtgcagagagaaaaaagagcagtgggaataggagctttgttcc
ttgggttcttgggagcagcaggaagcactatgggcgcagcgtcaatgacg
ctgacggtacaggccagacaattattgtctggtatagtgcagcagcagaa
caatttgctgaGGGCTATTGAGGCGCAACAGCATCTGTTGCAACTCACAG
TCTGGGGCATCAAGCAGCTCCAGGCAAGAATCCTGGCTGTGGAAAGATAC
CTAAAGGATCAACAGCTCCTGGGGATTTGGGGTTGCTCTGGAAAACTCAT
TTGCACCACTGCTGTGCCTTGGAATGCTAGTTGGAGTAATAAATCTCTGG
AACAGATTTGGAATCACACGACCTGGATGGAGTGGGACAGAGAAATTAAC
AATTACACAAGCTTAATACACTCCTTAATTGAAGAATCGCAAAACCAGCA
AGAAAAGAATGAACAAGAATTATTGGAATTAGATAAATGGGCAAGTTTGT
GGAATTGGTTTAACATAACAAATTGGCTGTGGTATATAAAATTATTCATA
ATGATAGTAGGAGGCTTGGTAGGTTTAAGAATAGTTTTTGCTGTACTTTC
TATAGTGAATAGAGTTAGGCAGGGATATTCACCATTATCGTTTCAGACCC
ACCTCCCAACCCCGAGGGGACCCGACAGGCCCGAAGGAATAGAAGAAGAA
GGTGGAGAGAGAGACAGAGACAGATCCATTCGATTAGTGAACGGATCTCG
ACGGTATCGGTTAACTTTTAAAAGAAAAGGGGGGATTGGGGGGTACAGTG
CAGGGGAAAGAATAGTAGACATAATAGCAACAGACATACAAACTAAAGAA
TTACAAAAACAAATTACAAAAATTCAAAATTTTATCG

LV-PL4-GTIIC::eGFP

GTIIC stiffness sensing promoter: Addgene 34615: 8×GTIIC-luciferase (Dupont, Nature, 2011)

Infusion Primers:

Infusion primers:
(SEQ ID NO: 17) Fw:
AATTTTATCGGGATCCCGAGCTCTTACGCGTGCTA
(SEQ ID NO: 18) Rv:
CGATGTATACGGATCCtttatATCGTCCCACGGAGCG
Enhanced green fluorescent protein (eGFP):
Addgene: 32548: pUCBB-eGFP
Promoterless vector (GenTarget, Inc Cat# LV-PL4)
(SEQ ID NO: 19)
GgatccCGAGCTCTTACGCGTGCTAGCCCGGGCTAGCCCGGCCAGTGCCA
AGTTGAGACACATTCCACACATTCCACTGCAAGCTTGAGACACATTCCAC
ACATTCCACTGCAAGCTTGGCCAGTGCCAAGTTGAGACACATTCCACACA
TTCCACTGCAAGCTTGAGACACATTCCACACATTCCACTGCAAGCTTCTA
GAGATCTGCAGGTCGAGGTCGACGGTATCGATAAGCTTGGGGGTGGGCGC
CGGGGGGACCTTAAAGCCTCTGCCCCCCAAGGAGCCCTTCCCAGACAGCC
GCCGGCACCCACCGCTCCGTGGGACGATataaagGATCCATATGGTGAGC
AAGGGCGAGGAGCTGTTCACCGGGGTGGTGCCCATCCTGGTCGAGCTGGA
CGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAG
CTGCCCGTGCCCTGGCCCACCCTCGTGACCACCCTGACCTACGGCGTGCA
GTGCTTCAGCCGCTACCCCGACCACATGAAGCAGCACGACTTCTTCAAGT
CCGCCATGCCCGAAGGCTACGTCCAGGAGCGCACCATCTTCTTCAAGGAC
GACGGCAACTACAAGACCCGCGCCGAGGTGAAGTTCGAGGGCGACACCCT
GGTGAACCGCATCGAGCTGAAGGGCATCGACTTCAAGGAGGACGGCAACA
TCCTGGGGCACAAGCTGGAGTACAACTACAACAGCCACAACGTCTATATC
ATGGCCGACAAGCAGAAGAACGGCATCAAGGTGAACTTCAAGATCCGCCA
CAACATCGAGGACGGCAGCGTGCAGCTCGCCGACCACTACCAGCAGAACA
CCCCCATCGGCGACGGCCCCGTGCTGCTGCCCGACAACCACTACCTGAGC
ACCCAGTCCGCCCTGAGCAAAGACCCCAACGAGAAGCGCGATCACATGGT
CCTGCTGGAGTTCGTGACCGCCGCCGGGATCACTCTCGGCATGGACGAGC
TGTACAAGTAAATGCATCCATGGCGGCCGCCTCGAGCACACCATCACCAT
CACTGATTCTCCTTACGCATCTGTGCGGTATTTCACACCGCATGTACTAG
TGTCGACGCTAGCTCTAGATGTACAAAGTGGTGCTAGCACTCTCAGTACA
ATCTGCTCTGATGCCGCATAGTTAAGCCAGTATCTGCTCCCTGCTTGTGT
GTTGGAGGTCGCTGAGTAGTGCGCGAGCAAAATTTAAGCTACAACAAGGC
AAGGCTTGACCGACAATTGCATGAAGAATCTGCTTAGGGTTAGGCGTTTT
GCGCTGCTTCGCGATGTACGGGCCAGATATACGCGTATCTGAGGGGACTA
GGGTGTGTTTAGGCGAAAAGCGGGGCTTCGGTTGTACGCGGTTAGGAGTC
CCCTCAGGATATAGTAGTTTCGCTTTTGCATAGGGAGGGGGAAATGTAGT
CTTATGCAATACTCTTGTAGTCTTGCAACATGGTAACGATGAGTTAGCAA
CATGCCTTACAAGGAGAGAAAAAGCACCGTGCATGCCGATTGGTGGAAGT
AAGGTGGTACGATCGTGCCTTATTAGGAAGGCAACAGACGGGTCTGACAT
GGATTGGACGAACCACTGAATTCCGCATTGCAGAGATATTGTATTTAAGT
GCCTAGCTCGATACAATAAACGCCATTTGACCATTCACCACATTGGTGTG
CACCTCCAAAGCGCTCACCATGACCGAGTACAAGCCCACGGTGCGCCTCG
CCACCCGCGACGACGTCCCCAGGGCCGTACGCACCCTCGCCGCCGCGTTC
GCCGACTACCCCGCCACGCGCCACACCGTCGATCCGGACCGCCACATCGA
GCGGGTCACCGAGCTGCAAGAACTCTTCCTCACGCGCGTCGGGCTCGACA
TCGGCAAGGTGTGGGTCGCGGACGACGGCGCCGCGGTGGCGGTCTGGACC
ACGCCGGAGAGCGTCGAAGCGGGGGCGGTGTTCGCCGAGATCGGCCCGCG
CATGGccgagttgagcggacccggctggccgcgcagcaacagatggaagg
cctcctggcgccgcaccggcccaaggagcccgcgtggacctggccaccgt
cggcgtctcgcccgaccaccagggcaagggtctgggcagcgccgtcgtgc
tccccggagtggaggcggccgagcgcgccggggtgcccgccttcctggag
acctccgcgccccgcaacctccccttctacgagcggctcggcttcaccgt
caccgccgacgtcgaggtgcccgaaggaccgcgcacctggtgcatgaccc
gcaagcccggtgcctgaactagttaggtttaaacacgcgtaccggttagt
aatgatcgacaatcaacctctggattacaaaatttgtgaaagattgactg
gtattcttaactatgttgctccttttacgctatgtggatacgctgcttta
atgcctttgtatcatgctattgcttcccgtatggctttcattttctcctc
cttgtataaatcctggttgctgtctctttatgaggagttgtggcccgttg
tcaggcaacgtggcgtggtgtgcactgtgtttgctgacgcaacccccact
ggttggggcattgccaccacctgtcagctcctttccgggactttcgcttt
ccccctccctattgccacggcggaactcatcgccgcctgccttgcccgct
gctggacaggggctcggctgagggcactgacaattccgtggtgttgtcgg
ggaagctgacgtcctttccatggctgctcgcctgtgttgccacctggatt
ctgcgcgggacgtccttctgctacgtcccttcggccctcaatccagcgga
ccttccttcccgcggcctgctgccggctctgcggcctcttccgcgtcttc
gccttcgccctcagacgagtcggatctccctttgggccgcctccccgcct
ggcgatggtacctttaagaccaatgacttacaaggcagctgtagatctta
gccactttttaaaagaaaaggggggactggaagggctaattcactcccaa
cgaagacaagatctgctttttgcttgtactgggtctctctggttagacca
gatctgagcctgggagctctctggctaactagggaacccactgcttaagc
ctcaataaagcttgccttgagtgcttcaagtagtgtgtgcccgtctgagt
gtgactctggtaactagagatccctcagacccttttagtcagtgtggaaa
atctctagcagtagtagttcatgtcatcttattattcagtatttataact
tgcaaagaaatgaatatcagagagtgagaggaacttgtttattgcagctt
ataatggttacaaataaagcaatagcatcacaaatttcacaaataaagca
tttttttcactgcattctagttgtggtagtccaaactcatcaatgtatct
tatcatgtctggctctagctatcccgcccctaactccgcccatcccgccc
ctaactccgcccagttccgcccattctccgccccatggctgactaatatt
tttatttatgcagaggccgaggccgcctcggcctctgagctattccagaa
gtagtgaggaggcttttttggaggcctagggacgtacccaattcgcccta
tagtgagtcgtattacgcgcgctcactggccgtcgttttacaacgtcgtg
actgggaaaaccctggcgttacccaacttaatcgccttgcagcacatccc
cctttcgccagctggcgtaatagcgaagaggcccgcaccgatcgcccttc
ccaacagttgcgcagcctgaatggcgaatgggacgcgccctgtagcggcg
cattaagcgcggcgggtgtggtggttacgcgcagcgtgaccgctacactt
gccagcgccctagcgcccgctcctttcgctacttcccaccatctcgccac
gttcgccggctttccccgtcaagctctaaatcgggggctccctttagggt
tccgatttagtgctttacggcacctcgaccccaaaaaacttgattagggt
gatggttcacgtagtgggccatcgccctgatagacggtttttcgcccttt
gacgttggagtccacgttctttaatagtggactcttgttccaaactggaa
caacactcaaccctatctcggtctattcttttgatttataagggattttg
ccgatttcggcctattggttaaaaaatgagctgatttaacaaaaatttaa
cgcgaattttaacaaaatattaacgcttacaatttaggtggcacttttcg
gggaaatgtgcgcggaacccctatagtttatttttctaaatacattcaaa
tatgtatccgctcatgagacaataaccctgataaatgcttcaataatatt
gaaaaaggaagagtatgagtattcaacataccgtgtcgcccttattccct
tttttgcggcattagccttcctgtattgctcacccagaaacgctggtgaa
agtaaaagatgctgaagatcagagggtgcacgagtgggttacatcgaact
ggatctcaacagcggtaagatccttgagagttttcgccccgaagaacgtt
ttccaatgatgagcacttttaaagttctgctatgtggcgcggtattatcc
cgtattgacgccgggcaagagcaactcggtcgccgcatacactattctca
gaatgacttggttgagtactcaccagtcacagaaaagcatcttacggatg
gcatgacagtaagagaattatgcagtgctgccataaccatgagtgataac
actgcggccaacttacttctgacaacgatcggaggaccgaaggagctaac
cgcttttttgcacaacatgggggatcatgtaactcgccttgatcgttggg
aaccggagctgaatgaagccataccaaacgacgagcgtgacaccacgatg
cctgtagcaatggcaacaacgttgcgcaaactattaactggcgaactact
tactctagcttcccggcaacaattaatagactggatggaggcggataaag
ttgcaggaccacttctgcgctcggcccttccggctggctggtttattgct
gataaatctggagccggtgagcgtgggtctcgcggtatcattgcagcact
ggggccagatggtaagccctcccgtatcgtagttatctacacgacgggga
gtcaggcaactatggatgaacgaaatagacagatcgctgagataggtgcc
tcactgattaagcattggtaactgtcagaccaagtttactcatatatact
ttagattgatttaaaacttcatttttaatttaaaaggatctaggtgaaga
tcctttttgataatctcatgaccaaaatcccttaacgtgagttacgacca
ctgagcgtcagaccccgtagaaaagatcaaaggatcttcttgagatcctt
tttttctgcgcgtaatctgctgcttgcaaacaaaaaaaccaccgctacca
gcggtggtagtagccggatcaagagctaccaactctttttccgaaggtaa
ctggcttcagcagagcgcagataccaaatactgttcttctagtgtagccg
tagttaggccaccacttcaagaactctgtagcaccgcctacatacctcgc
tctgctaatcctgttaccagtggctgctgccagtggcgataagtcgtgtc
ttaccgggttggactcaagacgatagttaccggataaggcgcagcggtcg
ggctgaacggggggttcgtgcacacagcccagcttggagcgaacgaccta
caccgaactgagatacctacagcgtgagctatagaaagcgccacgcttcc
cgaagggagaaaggcggacaggtatccggtaagcggcagggtcggaacag
gagagcgcacgagggagcttccagggggaaacgcctggtatctttatagt
cctgtcgggtacgccacctctgacttgagcgtcgatttttgtgatgctcg
tcaggggggcggagcctatggaaaaacgccagcaacgcggcctttttacg
gacctggccttagctggccttagctcacatgttctacctgcgttatcccc
tgattctgtggataaccgtattaccgcctttgagtgagctgataccgctc
gccgcagccgaacgaccgagcgcagcgagtcagtgagcgaggaagctgga
agggctaattcactcccaaagaagacaagatatccttgatctgtggatct
accacacacaaggctacttccctgattagcagaactacacaccagggcca
ggggtcagatatccactgacctttggatggtgctacaagctagtaccagt
tgagccagataaggtagaagaggccaataaaggagagaacaccagcttgt
tacaccctgtgagcctgcatgggatggatgacccggagagagaagtgtta
gagtggaggtttgacagccgcctagcatttcatcacgtggcccgagagct
gcatccggagtacttcaagaactgctgatatcgagcttgctacaagggac
tttccgctggggactttccagggaggcgtggcctgggcgggactggggag
tggcgagccctcagatcctgcatataagcagctgctttttgcctgtactg
ggtctctctggttagaccagatctgagcctgggagctctctggctaacta
gggaacccactgcttaagcctcaataaagcttgccttgagtgcttcaagt
agtgtgtgcccgtctgttgtgtgactctggtaactagagatccctcagac
ccttttagtcagtgtggaaaatctctagcagtggcgcccgaacagggact
tgaaagcgaaagggaaaccagaggagctctctcgacgcaggactcggctt
gctgaagcgcgcacggcaagaggcgaggggcggcgactggtgagtacgcc
aaaaattttgactagcggaggctagaaggagagagatgggtgcgagagcg
tcagtattaagcgggggagaattagatcgcgatgggaaaaaattcggtta
aggccagggggaaagaaaaaatataaattaaaacatatagtatgggcaag
cagggagctagaacgattcgcagttaatcctggcctgttagaaacatcag
aaggctgtagacaaatactgggacagctacaaccatcccttcagacagga
tcagaagaacttagatcattatataatacagtagcaaccctctattgtgt
gcatcaaaggatagagataaaagacaccaaggaagctttagacaagatag
aggaagagcaaaacaaaagtaagaccaccgcacagcaagcggccgctgat
cttcagacctggaggaggagatatgagggacaattggagaagtgaattat
ataaatataaagtagtaaaaattgaaccattaggagtagcacccaccaag
gcaaagagaagagtggtgcagagagaaaaaagagcagtgggaataggagc
tttgttccttgggttcttgggagcagcaggaagcactatgggcgcagcgt
caatgacgctgacggtacaggccagacaattattgtctggtatagtgcag
cagcagaacaatttgctgaGGGCTATTGAGGCGCAACAGCATCTGTTGCA
ACTCACAGTCTGGGGCATCAAGCAGCTCCAGGCAAGAATCCTGGCTGTGG
AAAGATACCTAAAGGATCAACAGCTCCTGGGGATTTGGGGTTGCTCTGGA
AAACTCATTTGCACCACTGCTGTGCCTTGGAATGCTAGTTGGAGTAATAA
ATCTCTGGAACAGATTTGGAATCACACGACCTGGATGGAGTGGGACAGAG
AAATTAACAATTACACAAGCTTAATACACTCCTTAATTGAAGAATCGCAA
AACCAGCAAGAAAAGAATGAACAAGAATTATTGGAATTAGATAAATGGGC
AAGTTTGTGGAATTGGTTTAACATAACAAATTGGCTGTGGTATATAAAAT
TATTCATAATGATAGTAGGAGGCTTGGTAGGTTTAAGAATAGTTTTTGCT
GTACTTTCTATAGTGAATAGAGTTAGGCAGGGATATTCACCATTATCGTT
TCAGACCCACCTCCCAACCCCGAGGGGACCCGACAGGCCCGAAGGAATAG
AAGAAGAAGGTGGAGAGAGAGACAGAGACAGATCCATTCGATTAGTGAAC
GGATCTCGACGGTATCGGTTAACTTTTAAAAGAAAAGGGGGGATTGGGGG
GTACAGTGCAGGGGAAAGAATAGTAGACATAATAGCAACAGACATACAAA
CTAAAGAATTACAAAAACAAATTACAAAAATTCAAAATTTTATCG

Other plasmids used in projects in the patent and the interesting sequences:

Firefly Luciferase-Red fluorescent protein (Fluc-RFP) fused protein: pcDNA3.1(+)/Luc2=tdT, Addgene 43904

Humanized Gaussia luciferase (hGluc): pSV40Gluc Plasmid, New England BioLabs, Inc. Cat #: N0323S.

Matrix Metallopeptidase 1 (MMP1): MMP1 (NM_002421) Human cDNA ORF Clone, Origene Technology, Inc Cat #: RG202460

Beta galactosidase: pOPINVL, Addgene 26040

### Example 7: Wound Healing

Wound healing is a complicated biological process that involves the coordination of many types of cells and factors to repair and restore damaged tissue. The normal response to injury typically involves three stages: inflammation, new tissue formation, and remodeling^{[95]}. During this process, the body works to stop infection, activates the migration and proliferation of different cells to repair the tissue, and remodels the new tissue to return the body to homeostasis. However, in injuries that have an underlying cause, such as diabetic foot ulcers, the normal healing process can be impaired^{[96]}. This leads to chronic wounds that do not heal.

Diabetic foot ulcers are a common instance of chronic wounds, caused by neuropathy and ischemia, and affecting as many as 25% of people with type 1 and type 2 diabetes in their lifetimes. Diabetic foot disease is also a leading cause of lower limb amputations^{[97]}. Due to the high rates of diabetes worldwide, the complications caused by diabetic foot ulcers present an important problem that needs to be addressed. Other than lifestyle changes, several current treatment methods include skin grafts, hyperbaric oxygen treatment, negative pressure dressings, and growth factor therapy. However, since diabetic foot ulcers have complex underlying causes, and are subject to constant mechanical stress, these treatments are often inadequate, thus leading to the need for amputation.

Mesenchymal stem cells (MSC) are a promising treatment for diabetic foot ulcers. MSC naturally secrete various factors that may help in the wound healing process, and have already been studied for use in human trials^{[98]}. However, these previous studies utilized unmodified autologous cells and the mechanisms of why would healing improved is still uncertain.

Provided are engineered MSC to elucidate the mechanisms of wound healing as well as enhancing the native effect of un-engineered cells; and in addition, since the ulcer region of plantar tissue has increased modulus^{[99]}, to selectively activate expression of healing factors in the wound by using a stiffness-sensing promoter. Provided are therapeutic genes for the engineered MSC to express is vascular endothelial growth factor (VEGF), which has been shown to improve angiogenesis and wound closure [100].

### Example 8: Mechano-Sensitive CAR T Cells

The large majority of clinical patients with solid tumor metastases, including those with known markers (such as HER2) will die from their disease^{[101]}. A unique approach to patient treatment is to utilize the patient's own immune system. One such approach, that has recently made it to clinical trials is to genetically modify T cells to target antigens expressed on tumor cells via the expression of chimeric antigen receptors (CARs), CARs are antigen receptors that are engineered to recognize cell surface antigens and activate T cells in an antigen-dependent manner^{[102]}. Attempts made to treat patients with solid tumor metastases using genetically modified cells expressing CARs have met with very limited success, and in some cases have been lethal^{[103, 104]}. Here we describe, how exemplary mechano-sensitive promoters as provided herein can be used to unique design CAR T-cell to target cancer in a logic dependent manner using logic-gated genetic circuits.

### Background

While CARs trigger T cell activation similarly to the endogenous T cell receptor a major limitation of this technology to clinical applications with respect solid tumor is the ability to express the CAR in specific tumor microenvironment. All previous CARs that have been described in the prior art utilize constitutive promoters^{[105-107]}. Therefore CARs are continuously expressed and always present on the T cell surface membrane after being genetically modified, even prior to infusion. Hence, when these CAR T cells bind to on-target off-tumor antigen they activate T cell responses in undesired locations in the patient that lead to lethal consequences.

Here we describe how by using mechano-responsive promoter logic, and the mechano-responsive promoters as provided herein, we can decrease the rate of on-target off-tumor T cell activation. Solid tumors whose surrounding ECM and the tumor microenvironment generally is remodeled with increased mechanical stiffness due to processes described above, such as cross-linking of collagen fibers. CAR-modified T-cells whose CAR is only expressed in such as specific tumor microenvironment will function as an AND-logic gate, as T cell activation will require both the presence of the tumor antigen (such as HER2/EGFRvIII^{[103, 108]}) and the presence of a tumor microenvironment with unique mechano-cues such as high mechanical stiffness.

An exemplary embodiment is described e.g., in FIG. 57. The process by which T cells are engineered is shown (FIG. 57A). Firstly the patient-derived T-cells are harvested, by apheresis, and isolated. Next, these T-cells are genetically engineered using, for example, either lentiviral constructs or using CRISPR/Cas9 for site-specific integration into T cells as has been shown previously^{[109]}. Lastly, these engineered T-cells are selected, expanded are re-infused into the patient. Once these cells are re-infused into the patient they are designed to decrease the on-target off-tumor specificity, and thus increase patient survivability, as is shown (FIGS. 57B and 57C).

In other embodiments, provided are complex logic-gates such as multi-input AND-gates or sequentially-stage AND-gates; optionally using methods described previously and already shown in T cells^{[110, 111]}. These methods can be used to engineer T cells whose CAR requires the presence of multi specific stimuli in the local tumor microenvironment. For example, collagen crosslinking can be targeted specifically via synthetic receptors that target LOXL1 and/or LOXL2. Single-chain antibodies, specific for these enzymes have been found previously^{[112, 113]}.

As an example, synthetic receptors are created by fusing three main components in a modular fashion. Firstly, the target enzyme is detected via a single-chain variable fragment domain (scFv). Then, a transmembrane domain from mouse Notch protein target the receptor to the membrane and allows for specific intracellular cleavage upon scFv binding of the third domain. This last domain is the transcriptional activator of downstream components of the genetic circuit. These transcriptional switching proteins are varied and can range from ones that permanently switch circuits between states, to transient activators that only allow transcriptional activation upon constant input signal. In the first category are serine-integrase (such as Bxb1/PhiC31), these can be used to remove site specifically remove genomic segments that repress the expression of desired target proteins (as is used above in Example 6). In the second category are trans-activators such as the GAL4DBD system also described above, but this time fused to a transactivating VP16 domain [91].

Other biophysical stimuli can be such as hypoxia, and oxidative stress can be target using novel modified synthetic promoters and these promoters can be coupled to create even more complex logic-gated CAR T cells, that only activate in biophysically constrained tumor microenvironments with certain stiffness, collagen cross-linking, oxygen concentration and nitric oxide synthase and reactive oxygen species (ROS/NOS) concentration. Targeting these biophysical cues can be used in combination with engineering cells to target other signals especially the biochemical cues. Together, the embodiments as provided herein reported here enable designing cells that can target biophysical and/or biochemical or other signals associated or surrounding cells to effectively treat a disease with minimized side effects.

### Example 9: Mechano-Sensitive Transgenic Mice

Non-human transgenic animal models are useful for screening drugs and are commonly used as research models of developmental processes. This example describes use of an exemplary engineered non-human animal as described herein, whose cells are modified with a genetic circuit that reports on the mechano-sensitive nature of its local environment. Such a circuit has the utility of being able to report, using reporting molecules or devices such as fluorescent proteins, the biophysical properties (e.g., varying stiffness) present during the development of the animal and the ongoing mechanical state of the microenvironment.

### Background

Mechanobiology has been described above as an emerging field. The primary form in which this field has been studied is using engineered in vitro models or wild-type animal models. That is the mechanical properties of cells and how cells react to substrates of varying stiffness have been investigated using engineered cells that have been grown in tissue culture such as in precise experiments described previously^{[114], [115]}.

Contrastingly, while it is has been common to study the mechanical properties of animal tissue, no non-human animal have been engineered with the specific purpose of studying the mechanobiology of the entire animal. Whereas previous physiological research was primarily focused on biochemical pathways, it would be uniquely useful to study mechanical properties of all tissues as these properties have been shown to be central to development, function, and disease states. The ability to detect tissue stiffness in vivo in the native animal context can be a powerful tool to study mechanobiology and as a model to evaluate methods that perturb the mechano-niche. Previously transgenic animal models have been created and patented to study oncogenes^{[116]} and the generation of antibodies^{[117]}.

The current tools used to study mechanobiology of in vivo animals models falls into two categories: imaging and mechanical testing, as described above in Example 6. The same tools have that have been developed for in vitro tissue culture have been used for in vivo animals models, such as different in vivo imaging modalities, ex vivo mechanical testing of tissue. However, no tool exists to correlate in vivo imaging measurements to values of mechanical stiffness.

In alternative embodiments, cells are engineered with many different promoters in genetic circuits that selectively activate on substrates of varying stiffness as described in Example 6 and elsewhere above. Similar genetic circuits have been widely integrated into many cells types^{[92]} and animal models using site-specific integration methods^{[118]}. Such genetic circuits are engineered site-specifically into non-human animal models.

### Experimental Design:

Engineered mechano-sensitive transgenic mice for to sensing stiffness in vivo are provided, and in alternative embodiments, are made using engineered genetic circuits described in Example 6 above, specifically both the two state and multi-state variants described (FIGS. 55 and 56). These circuits are constructed using a slightly modified version of the modular vector assembly approach described previously^{[92]}. The plasmid constructs are modified such that CRISPR/Cas9 initiated homology directed repair is targeted to the mROSA26 locus on the mouse genome^{[94]}. These genetic constructs are then inserted into C57BL/6 mice using previously described protocols^{[118]}.

Validate Mechano-Sensitive Circuits In Vivo

To validate the mechano-sensitive reporter system in vivo, a standard curve can first be constructed using engineered cells cultured on collagen-coated polyacrylamide hydrogels. Varying the relative concentrations of acrylamide and bis-acrylamide creates hydrogels with tunable stiffness. Hydrogels with multiple known stiffness ranges are formed covering a range from (0.1 to 40 kPa). Stably expressing cells engineered with mechano-sensitive genetic circuits are cultured on collagen-coated hydrogels of varying stiffness and imaged using a standard epifluorescent microscope. Average expression of each fluorescent reporter is measured for thousands of cells. The average expression value and standard deviation over all cells is used to construct a distribution of fluorescent intensity values for each substrate stiffness value in the range. This distribution is the used to construct a standard curve of how the various fluorescent protein intensities vary with differing stiffness. This standard curve is then used to correlate known fluorescent intensity values from the in vivo mouse model to known mechanical stiffness values.

Then, sectioned mouse tissue samples are imaged to calculate and to measure the variation in fluorescent intensity across entire tissues sections of different organs. The fluorescent protein intensity across each tissue is converted into a stiffness “map” of each section and is used to construct a stiffness “3D-model” of the entire mouse. This data is validated against in vivo measurements from traditional mechanical-testing methods, such as atomic force microscopy (AFM) across each tissue section.

### REFERENCES

1. Sander, J. D. and J. K. Joung, CRISPR-Cas systems for editing, regulating and targeting genomes. Nat Biotech, 2014. 32(4): p. 347-355.
2. Liu, L., et al., From blood to the brain: can systemically transplanted mesenchymal stem cells cross the blood-brain barrier? Stem Cells Int, 2013. 2013: p. 435093.
3. Park, J. S., et al., The effect of matrix stiffness on the differentiation of mesenchymal stem cells in response to TGF-beta. Biomaterials, 2011. 32(16): p. 3921-30.
4. Engler, A. J., et al., Matrix elasticity directs stem cell lineage specification. Cell, 2006. 126(4): p. 677-89.
5. Hanahan, D. and R. A. Weinberg, Hallmarks of cancer: the next generation. Cell, 2011. 144(5): p. 646-74.
6. Hedley, B. D. and A. F. Chambers, Tumor dormancy and metastasis. Adv Cancer Res, 2009. 102: p. 67-101.
7. Levental, K. R., et al., Matrix crosslinking forces tumor progression by enhancing integrin signaling. Cell, 2009. 139(5): p. 891-906.
8. Leight, J. L., et al., Matrix rigidity regulates a switch between TGF-beta1-induced apoptosis and epithelial-mesenchymal transition. Mol Biol Cell, 2012. 23(5): p. 781-91.
9. Wong, C. C., et al., Hypoxia-inducible factor 1 is a master regulator of breast cancer metastatic niche formation. Proc Natl Acad Sci USA, 2011. 108(39): p. 16369-74.
10. Erler, J. T., et al., Lysyl oxidase is essential for hypoxia-induced metastasis. Nature, 2006. 440(7088): p. 1222-6.
11. Erler, J. T., et al., Hypoxia-induced lysyl oxidase is a critical mediator of bone marrow cell recruitment to form the premetastatic niche. Cancer Cell, 2009. 15(1): p. 35-44.
12. Bondareva, A., et al., The lysyl oxidase inhibitor, beta-aminopropionitrile, diminishes the metastatic colonization potential of circulating breast cancer cells. PLoS One, 2009. 4(5): p. e5620.
13. Wong, C. C., et al., Inhibitors of hypoxia-inducible factor 1 block breast cancer metastatic niche formation and lung metastasis. J Mol Med (Berl), 2012. 90(7): p. 803-15.
14. McPherson, J. M., S. J. Sawamura, and A. Conti, Preparation of [3H]collagen for studies of the biologic fate of xenogenic collagen implants in vivo. J Invest Dermatol, 1986. 86(6): p. 673-7.
15. Ankrum, J. and J. M. Karp, Mesenchymal stem cell therapy: Two steps forward, one step back. Trends Mol Med, 2010. 16(5): p. 203-9.
16. Karp, J. M. and G. S. Leng Teo, Mesenchymal stem cell homing: the devil is in the details. Cell Stem Cell, 2009. 4(3): p. 206-16.
17. Trounson, A., et al., Clinical trials for stem cell therapies. BMC Med, 2011. 9: p. 52.
18. Wang, H., et al., Trafficking mesenchymal stem cell engraftment and differentiation in tumor-bearing mice by bioluminescence imaging. Stem Cells, 2009. 27(7): p. 1548-58.
19. Reagan, M. R. and D. L. Kaplan, Concise review: Mesenchymal stem cell tumor-homing: detection methods in disease model systems. Stem Cells, 2011. 29(6): p. 920-7.
20. Tse, J. R. and A. J. Engler, Stiffness gradients mimicking in vivo tissue variation regulate mesenchymal stem cell fate. PLoS One, 2011. 6(1): p. e15978.
21. Samani, A., J. Zubovits, and D. Plewes, Elastic moduli of normal and pathological human breast tissues: an inversion-technique-based investigation of 169 samples. Phys Med Biol, 2007. 52(6): p. 1565-1576.
22. Studeny, M., et al., Bone marrow-derived mesenchymal stem cells as vehicles for interferon-beta delivery into tumors. Cancer Res, 2002. 62(13): p. 3603-8.
23. Aboody, K. S., et al., Targeting of melanoma brain metastases using engineered neural stem/progenitor cells. Neuro Oncol, 2006. 8(2): p. 119-26.
24. Aboody, K. S., et al., Neural stem cell-mediated enzyme/prodrug therapy for glioma: preclinical studies. Sci Transl Med, 2013. 5(184): p. 184ra59.
25. Brader, P., et al., Imaging of lymph node micrometastases using an oncolytic herpes virus and [F]FEAU PET. PLoS One, 2009. 4(3): p. e4789.
26. Whitney, M. A., et al., Fluorescent peptides highlight peripheral nerves during surgery in mice. Nat Biotechnol, 2011. 29(4): p. 352-6.
27. Hussain, T. and Q. T. Nguyen, Molecular imaging for cancer diagnosis and surgery. Adv Drug Deliv Rev, 2014. 66: p. 90-100.
28. Mehlen, P. and A. Puisieux, Metastasis: a question of life or death. Nat Rev Cancer, 2006. 6(6): p. 449-58.
29. Kapoor, V., B. M. McCook, and F. S. Torok, An introduction to PET-CT imaging. Radiographics, 2004. 24(2): p. 523-43.
30. Schick, F., Whole-body MRI at high field: technical limits and clinical potential. Eur Radiol, 2005. 15(5): p. 946-59.
31. Buchberger, W., et al., Clinically and mammographically occult breast lesions: detection and classification with high-resolution sonography. Semin Ultrasound CT MR, 2000. 21(4): p. 325-36.
32. Hanash, S. M., C. S. Baik, and O. Kallioniemi, Emerging molecular biomarkers—blood-based strategies to detect and monitor cancer. Nat Rev Clin Oncol, 2011. 8(3): p. 142-50.
33. Warren, A. D., et al., Disease detection by ultrasensitive quantification of microdosed synthetic urinary biomarkers. J Am Chem Soc, 2014. 136(39): p. 13709-14.
34. Kwong, G. A., et al., Mass-encoded synthetic biomarkers for multiplexed urinary monitoring of disease. Nat Biotechnol, 2013. 31(1): p. 63-70.
35. Schroder, F. H., et al., Prostate-cancer mortality at 11 years of follow-up. N Engl J Med, 2012. 366(11): p. 981-90.
36. Campeau, P. M., W. D. Foulkes, and M. D. Tischkowitz, Hereditary breast cancer: new genetic developments, new therapeutic avenues. Human genetics, 2008. 124(1): p. 31-42.
37. Easton, D. F., How many more breast cancer predisposition genes are there. Breast Cancer Res, 1999. 1(1): p. 14-17.
38. Tomasetti, C. and B. Vogelstein, Cancer etiology. Variation in cancer risk among tissues can be explained by the number of stem cell divisions. Science, 2015. 347(6217): p. 78-81.
39. Loebinger, M. R. and S. M. Janes, Stem cells as vectors for antitumour therapy. Thorax, 2010. 65(4): p. 362-9.
40. Studeny, M., et al., Mesenchymal stem cells: potential precursors for tumor stroma and targeted-delivery vehicles for anticancer agents. J Natl Cancer Inst, 2004. 96(21): p. 1593-603.
41. Shah, K., Mesenchymal stem cells engineered for cancer therapy. Adv Drug Deliv Rev, 2012. 64(8): p. 739-48.
42. Droujinine, I. A., M. A. Eckert, and W. Zhao, To grab the stroma by the horns: from biology to cancer therapy with mesenchymal stem cells. Oncotarget, 2013. 4(5): p. 651-64.
43. Kidd, S., et al., Direct evidence of mesenchymal stem cell tropism for tumor and wounding microenvironments using in vivo bioluminescent imaging. Stem Cells, 2009. 27(10): p. 2614-23.
44. Zhao, W., et al., Mesenchymal stem cell biodistribution, migration, and homing in vivo. Stem Cells Int, 2014. 2014: p. 292109.
45. Ankrum, J. A., J. F. Ong, and J. M. Karp, Mesenchymal stem cells: immune evasive, not immune privileged. Nat Biotechnol, 2014. 32(3): p. 252-60.
46. Phinney, D. G. and D. J. Prockop, Concise review: Mesenchymal stem/multipotent stromal cells: The state of transdifferentiation and modes of tissue repair—Current views. Stem Cells, 2007. 25(11): p. 2896-2902.
47. Bexell, D., S. Scheding, and J. Bengzon, Toward brain tumor gene therapy using multipotent mesenchymal stromal cell vectors. Mol Ther, 2010. 18(6): p. 1067-75.
48. Hingtgen, S. D., et al., A novel molecule integrating therapeutic and diagnostic activities reveals multiple aspects of stem cell-based therapy. Stem Cells, 2010. 28(4): p. 832-41.
49. Chung, E., et al., Secreted Gaussia luciferase as a biomarker for monitoring tumor progression and treatment response of systemic metastases. PLoS One, 2009. 4(12): p. e8316.
50. Tannous, B. A. and J. Teng, Secreted blood reporters: insights and applications. Biotechnol Adv, 2011. 29(6): p. 997-1003.
51. Bovenberg, M. S., M. H. Degeling, and B. A. Tannous, Enhanced Gaussia luciferase blood assay for monitoring of in vivo biological processes. Anal Chem, 2012. 84(2): p. 1189-92.
52. El-Amouri, S. S., et al., Secreted luciferase for in vivo evaluation of systemic protein delivery in mice. Mol Biotechnol, 2013. 53(1): p. 63-73.
53. Dull, T., et al., A third-generation lentivirus vector with a conditional packaging system. J Virol, 1998. 72(11): p. 8463-71.
54. Tannous, B. A., Gaussia luciferase reporter assay for monitoring biological processes in culture and in vivo. Nat Protoc, 2009. 4(4): p. 582-91.
55. Donovan, J. and P. Brown, Blood collection. Curr Protoc Neurosci, 2005. Appendix 4: p. Appendix 4G.
56. Kong, Y., et al., Whole-body imaging of infection using bioluminescence. Curr Protoc Microbiol, 2011. Chapter 2: p. Unit 2C 4.
57. Tannous, B. A., et al., Codon-optimized Gaussia luciferase cDNA for mammalian gene expression in culture and in vivo. Mol Ther, 2005. 11(3): p. 435-43.
58. Welsh, J. P., et al., Multiply mutated Gaussia luciferases provide prolonged and intense bioluminescence. Biochem Biophys Res Commun, 2009. 389(4): p. 563-8.
59. Fantozzi, A. and G. Christofori, Mouse models of breast cancer metastasis. Breast Cancer Res, 2006. 8(4): p. 212.
60. Wang, C. Y. and P. H. Brown, Animal Models for Breast Cancer Prevention Research. Genetically Engineered Mice for Cancer Research: Design, Analysis, Pathways, Validation and Pre-Clinical Testing, 2012: p. 497-526.
61. Close, D. M., et al., In vivo bioluminescent imaging (BLI): noninvasive visualization and interrogation of biological processes in living animals. Sensors (Basel), 2011. 11(1): p. 180-206.
62. Prescher, J. A. and C. H. Contag, Imaging Mouse Models of Human Cancer. Genetically Engineered Mice for Cancer Research: Design, Analysis, Pathways, Validation and Pre-Clinical Testing, 2012: p. 235-260.
63. Caplan, A. I. and D. Correa, The MSC: an injury drugstore. Cell Stem Cell, 2011. 9(1): p. 11-5.
64. El-Haibi, C. P., et al., Critical role for lysyl oxidase in mesenchymal stem cell-driven breast cancer malignancy. Proc Natl Acad Sci USA, 2012. 109(43): p. 17460-5.
65. Li, H. J., et al., Cancer-stimulated mesenchymal stem cells create a carcinoma stem cell niche via prostaglandin E2 signaling. Cancer Discov, 2012. 2(9): p. 840-55.
66. Usunier, B., et al., Management of Fibrosis: The Mesenchymal Stromal Cells Breakthrough. Stem Cells Int, 2014. 2014.
67. Love, R. J. and K. S. Jones, Biomaterials, fibrosis, and the use of drug delivery systems in future antifibrotic strategies. Critical Reviews™ in Biomedical Engineering, 2009. 37(3).
68. Iimuro, Y. and D. A. Brenner, Matrix metalloproteinase gene delivery for liver fibrosis. Pharmaceutical research, 2008. 25(2): p. 249-258.
69. Kaar, J. L., et al., Matrix metalloproteinase-1 treatment of muscle fibrosis. Acta biomaterialia, 2008. 4(5): p. 1411-1420.
70. Foronjy, R. F., et al., Transgenic expression of matrix metalloproteinase-1 inhibits myocardial fibrosis and prevents the transition to heart failure in a pressure overload mouse model. Hypertension Research, 2008. 31(4): p. 725.
71. Ward, E., et al., Childhood and adolescent cancer statistics, 2014. CA: A Cancer Journal for Clinicians, 2014. 64(2): p. 83-103.
72. Mallhi, K., et al., Hematopoietic Cell Transplantation and Cellular Therapeutics in the Treatment of Childhood Malignancies. Pediatric Clinics of North America, 2015. 62(1): p. 257-273.
73. Woodard, P., et al., Etiology and Outcome of Graft Failure in Pediatric Hematopoietic Stem Cell Transplant Recipients. Journal of Pediatric Hematology/Oncology, 2003. 25(12): p. 955-959.
74. Locatelli, F., et al., Hematopoietic stem cell transplantation (HSCT) in children with juvenile myelomonocytic leukemia (JMMIL): results of the EWOG-MDS/EBMT trial. Vol. 105. 2005. 410-419.
75. Clark, J. R., et al., Monitoring of chimerism following allogeneic haematopoietic stem cell transplantation (HSCT): Technical recommendations for the use of Short Tandem Repeat (STR) based techniques, on behalf of the United Kingdom National External Quality Assessment Service for Leucocyte Immunophenotyping Chimerism Working Group. British Journal of Haematology, 2015. 168(1): p. 26-37.
76. Hussain, T. and Q. T. Nguyen, Molecular imaging for cancer diagnosis and surgery. Advanced Drug Delivery Reviews, 2014. 66(0): p. 90-100.
77. Kang, D.-K., et al., Droplet microfluidics for single-molecule and single-cell analysis in cancer research, diagnosis and therapy. TrAC Trends in Analytical Chemistry, 2014. 58(0): p. 145-153.
78. Rondelez, Y., et al., Microfabricated arrays of femtoliter chambers allow single molecule enzymology. Nat Biotech, 2005. 23(3): p. 361-365.
79. Kang, J. H. and J.-K. Chung, Molecular-Genetic Imaging Based on Reporter Gene Expression. Journal of Nuclear Medicine, 2008. 49(Suppl 2): p. 164S-179S.
80. Zhang, Y. and W. M. Pardridge, Delivery of β-Galactosidase to Mouse Brain via the Blood-Brain Barrier Transferrin Receptor. Journal of Pharmacology and Experimental Therapeutics, 2005. 313(3): p. 1075-1081.
81. Pelisek, J., S. Armeanu, and S. Nikol, Evaluation of β-Galactosidase Activity in Tissue in the Presence of Blood. Journal of Vascular Research, 2000. 37(6): p. 585-593.
82. McGarrity, G. J., et al., Patient monitoring and follow-up in lentiviral clinical trials. The Journal of Gene Medicine, 2013. 15(2): p. 78-82.
83. Kalos, M., et al., T Cells with Chimeric Antigen Receptors Have Potent Antitumor Effects and Can Establish Memory in Patients with Advanced Leukemia. Science Translational Medicine, 2011. 3(95): p. 95ra73-95ra73.
84. Busch, K., et al., Fundamental properties of unperturbed haematopoiesis from stem cells in vivo. Nature, 2015. 518(7540): p. 542-546.
85. Inlay, Matthew A., et al., Identification of Multipotent Progenitors that Emerge Prior to Hematopoietic Stem Cells in Embryonic Development. Stem Cell Reports, 2014. 2(4): p. 457-472.
86. Fathman, John W., et al., Upregulation of CD11A on Hematopoietic Stem Cells Denotes the Loss of Long-Term Reconstitution Potential. Stem Cell Reports, 2014. 3(5): p. 707-715.
87. Samani, A., J. Zubovits, and D. Plewes, Elastic moduli of normal and pathological human breast tissues: an inversion-technique-based investigation of 169 samples. Physics in medicine and biology, 2007. 52(6): p. 1565.
88. Engler, A. J., et al., Matrix elasticity directs stem cell lineage specification. Cell, 2006. 126(4): p. 677-689.
89. Szymczak, A. L., et al., Correction of multi-gene deficiency in vivo using a single ‘self-cleaving’2A peptide-based retroviral vector. Nature biotechnology, 2004. 22(5): p. 589-594.
90. Bintu, L., et al., Dynamics of epigenetic regulation at the single-cell level. Science, 2016. 351(6274): p. 720-724.
91. Feany, M. B. and W. W. Bender, A Drosophila model of Parkinson's disease. Nature, 2000. 404(6776): p. 394-398.
92. Guye, P., et al., Rapid, modular and reliable construction of complex mammalian gene circuits. Nucleic acids research, 2013: p. gkt605.
93. Liu, M., et al., Genomic discovery of potent chromatin insulators for human gene therapy. Nature biotechnology, 2015. 33(2): p. 198-203.
94. Sadelain, M., E. P. Papapetrou, and F. D. Bushman, Safe harbours for the integration of new DNA in the human genome. Nat Rev Cancer, 2012. 12(1): p. 51-58.
95. Gurtner, G. C., et al., Wound repair and regeneration. Nature, 2008. 453(7193): p. 314-321.
96. Falanga, V., Wound healing and its impairment in the diabetic foot. The Lancet, 2005. 366(9498): p. 1736-1743.
97. Boulton, A. J., et al., The global burden of diabetic foot disease. The Lancet, 2005. 366(9498): p. 1719-1724.
98. Dash, N. R., et al., Targeting nonhealing ulcers of lower extremity in human through autologous bone marrow-derived mesenchymal stem cells. Rejuvenation research, 2009. 12(5): p. 359-366.
99. Pai, S. and W. R. Ledoux, The compressive mechanical properties of diabetic and non-diabetic plantar soft tissue. Journal of biomechanics, 2010. 43(9): p. 1754-1760.
100. Brem, H., et al., Mechanism of sustained release of vascular endothelial growth factor in accelerating experimental diabetic healing. Journal of Investigative Dermatology, 2009. 129(9): p. 2275-2287.
101. Hinrichs, C. S. and S. A. Rosenberg, Exploiting the curative potential of adoptive T-cell therapy for cancer. Immunological reviews, 2014. 257(1): p. 56-71.
102. Kochenderfer, J. N., et al., B-cell depletion and remissions of malignancy along with cytokine-associated toxicity in a clinical trial of anti-CD19 chimeric-antigen-receptor-transduced T cells. Blood, 2012. 119(12): p. 2709-2720.
103. Ahmed, N., et al., Regression of experimental medulloblastoma following transfer of HER2-specific T cells. Cancer research, 2007. 67(12): p. 5957-5964.
104. Morgan, R. A., et al., Case report of a serious adverse event following the administration of T cells transduced with a chimeric antigen receptor recognizing ERBB2. Molecular Therapy, 2010. 18(4): p. 843-851.
105. Jensen, M., Chimeric immunoreceptor useful in treating human gliomas. 2009, Google Patents.
106. Jensen, M., Method and compositions for enhanced anti-tumor effector functioning oft cells. 2010, Google Patents.
107. Sadelain, M., R. Brentjens, and J. Maher, Chimeric T cell receptors. 2004, Google Patents.
108. Morgan, R. A., et al., Recognition of glioma stem cells by genetically modified T cells targeting EGFRvIII and development of adoptive cell therapy for glioma. Human gene therapy, 2012. 23(10): p. 1043-1053.
109. Schumann, K., et al., Generation of knock-in primary human T cells using Cas9 ribonucleoproteins. Proceedings of the National Academy of Sciences, 2015. 112(33): p. 10437-10442.
110. Roybal, Kole T., et al., Precision Tumor Recognition by T Cells With Combinatorial Antigen-Sensing Circuits. Cell. 164(4): p. 770-779.
111. Morsut, L., et al., Engineering Customized Cell Sensing and Response Behaviors Using Synthetic Notch Receptors. Cell. 164(4): p. 780-791.
112. McCauley, S. A., et al., Antibodies that bind to lysyl oxidase-like 2 (LOXL2). 2014, Google Patents.
113. Smith, V., et al., Methods and compositions inhibiting a lysyl oxidase (-like) protein for treatment and diagnosis of fibrosis, tumor invasion, angiogenesis, and metastasis. 2013, Google Patents.
114. Dupont, S., et al., Role of YAP/TAZ in mechanotransduction. Nature, 2011. 474(7350): p. 179-183.
115. Engler, A. J., et al., Myotubes differentiate optimally on substrates with tissue-like stiffness: pathological implications for soft or stiff microenvironments. The Journal of Cell Biology, 2004. 166(6): p. 877-887.
116. Leder, P. and T. A. Stewart, Recombinant activated oncogene; early transgenic animal patent. 1988, Google Patents.
117. Kucherlapati, R. and A. Jakobovits, Method of making transgenic mice lacking endogenous heavy chains. 1999, Google Patents.
118. Yang, H., H. Wang, and R. Jaenisch, Generating genetically modified mice using CRISPR/Cas-mediated genome engineering. Nature protocols, 2014. 9(8): p. 1956-1968.

A number of embodiments of the invention have been described. Nevertheless, it will be understood that various modifications may be made without departing from the spirit and scope of the invention. Accordingly, other embodiments are within the scope of the following claims.

1: An engineered or recombinant cell
comprising:
(1) (a) providing a cell, or engineering method that changes the content of the cell to generate the engineered cell, and modifying the cell to comprise, include or have contained therein, or have the ability to express: a therapeutic agent, a converter enzyme, a pro-enzyme, an antibody, an exogenous protein, an exogenous nanoparticle, a homing agent, or any molecule or device that originally does not exist in the cell, or
(b) (the engineered or recombinant cell) comprising, includes or has contained therein, or is modified to have the ability to express: a therapeutic agent, a converter enzyme, a pro-enzyme, an antibody, an exogenous protein, an exogenous nanoparticle, a homing agent, or any molecule or device that originally does not exist in the cell,
or
(2) (a) providing an engineered or recombinant cell having a modified cellular content or comprising an exogenous factor to modify the cell's physiology, or biochemical or biophysical mechanisms, for differentiation, homing, mechano-signals, cell-cell communication, soluble factors, extracellular environment, or response to other factors, or
(b) (the engineered or recombinant cell) comprising, includes or has contained therein a modified cellular content or comprising an exogenous factor to modify the cell's physiology, or biochemical or biophysical mechanisms, for differentiation, homing, mechano-signals, cell-cell communication, soluble factors, extracellular environment, or response to other factors,
wherein optionally the exogenous factor to modify the cell's physiology, or biochemical or biophysical mechanisms, for differentiation, homing, mechano-signals, cell-cell communication, soluble factors, extracellular environment, or response to other factors is encoded by a nucleic acid under the control of (operably linked to) a mechanoresponsive promoter, optionally a YAP/TAZ mechanoresponsive promoter,
and optionally the modified mechanism of differentiation of the engineered or recombinant cell alters its location and cellular content upon changing the cellular type specificity from low to high,
and optionally the modified mechano-signaling of the engineered or recombinant cell alters its location and cellular content upon receiving a stiffness and/or crosslinking signal from extracellular matrix or extracellular environment,
and optionally the modified mechanism for homing of the engineered or recombinant cell comprises homing to certain niche,
and optionally the modified mechanism of cell-cell communication of the engineered cell alters its location and cellular content upon interacting with other cells, and optionally the modified generation of soluble factors by the engineered or recombinant cell alters its location and cellular content upon receiving factors in the extracellular environment,
and optionally the modified extracellular environment of the engineered or recombinant cell alters its location and cellular content in response to the content in the extracellular environment,
and optionally the modified chemical condition of the engineered or recombinant cell alters the location and/or cellular content of the engineered cell, wherein optionally the modified engineered cell comprises proteins, nucleic acids, lipids, carbohydrates, small molecules, pH, temperature, radiation, or any other factor for altering the location of the cell; or
(3) (a) providing an engineered or recombinant cell as set forth in steps (1) or (2) above, wherein the cell is modified to have, comprise or contain therein at least one exogenous nucleic acid having the ability to express: a therapeutic agent, a converter enzyme, a pro-enzyme, an antibody, an exogenous protein, an exogenous nanoparticle, a homing agent, or any molecule that originally does not exist in the cell, and expression of the nucleic acid is constitutive or activatable (inducible), or
(b) an engineered or recombinant cell as set forth in steps (1) or (2) above, wherein the engineered cell is modified to have, comprise or contain therein at least one exogenous nucleic acid having the ability to express: a therapeutic agent, a converter enzyme, a pro-enzyme, an antibody, an exogenous protein, an exogenous nanoparticle, a homing agent, or any molecule that originally does not exist in the cell, and expression of the nucleic acid is constitutive or activatable (inducible),
wherein optionally the exogenous nucleic acid under the control of (operably linked to) a mechano-responsive promoter, optionally a YAP/TAZ mechanoresponsive promoter,
wherein optionally the constitutive expression persists regardless of the extracellular environment.
wherein optionally the activatable or inducible expression begins upon a mechanism described in (2), or is activatable or inducible by expression of an exogenous factor to modify the cell's physiology, or a biochemical or biophysical mechanism, or expression of a factor for differentiation, homing, mechano-signaling, cell-cell
communication, exposure to a soluble factor or an extracellular environment, or response to other factors,
wherein optionally the cell engineering is by a method comprising a genetic method, optionally CRISPR/Cas9 method or equivalent, or a non-genetic method; or
(4) (a) providing an engineered or recombinant cell that enables treatment of a disease or condition through the expression of a converter enzyme, a direct therapeutic enzyme, a pro-enzyme, an antibody, or any molecule that directly or indirectly aids in the therapeutic process, or
(b) (the engineered or recombinant cell) comprising, includes or has contained therein a converter enzyme, a therapeutic enzyme, a pro-enzyme, an antibody, or any molecule that directly or indirectly aids in the therapeutic process,
wherein optionally the converter enzyme, therapeutic enzyme, pro-enzyme, antibody, or molecule that directly or indirectly aids in the therapeutic process is encoded by an exogenous or an endogenous nucleic acid under the control of (operably linked to) a mechano-responsive promoter, optionally a YAP/TAZ mechanoresponsive promoter, and optionally the endogenous nucleic acid is engineered to be operably linked to a mechano-responsive promoter by a CRISPR/Cas9 methodology or equivalent,
wherein optionally the treatment comprises use of a converter enzyme or any protein or any other molecule that converts an inactive form of therapeutic agent into its active form,
and optionally the treatment comprises use of a direct therapeutic enzyme that directs alteration of the content of a cell or an extracellular environment,
and optionally the treatment comprises use of a pro-enzyme or any protein or any molecule produced by the engineered cell, wherein its form is altered from inactive to active in response to mechanisms described in (2), and delivers a therapeutic effect in its active form,
and optionally the treatment comprises use of an antibody or immunoglobulin produced by the engineered cell, which aids in the therapeutic process directly or indirectly; or
(5) (a) providing an engineered or recombinant cell that enables an assay for detection or diagnostics, companion diagnostics, or scientific and research tools, or
(b) (the engineered cell) comprising a nucleic acid encoding a protein that enables detection of the cell, or enables detection of the cell when the cell is exposed to a new environment, optionally a tissue or environment having an increased mechanical modulus, or stiffness, optionally the nucleic acid is under the control of (operably linked to) a mechano-responsive promoter, optionally a YAP/TAZ mechanoresponsive promoter,
wherein optionally the utility, assay for detection or diagnostics comprises of in vitro, in vivo, ex vivo, in situ or any other form of assay that enables the detection of the cellular location and/or content of the engineered cell,
and optionally the utility, companion diagnostics comprises of equipment and/or platform that enables the detection of cellular location and/or content of the engineered cells,
and optionally the utility, companion diagnostics comprises of equipment and/or platform that enable cell fate tracking and monitoring by detecting probes (e.g., enzymes) secreted by the cell into biological fluids including e.g., blood and urine,
and optionally the probes can be the therapeutic itself (e.g., a gene or a protein) in the case of gene cell therapy or other molecules or agents engineered into the cell,
and optionally the utility, companion diagnostics comprises of equipment and/or platform that permits single molecule detection from biological samples,
and optionally the utility, scientific and/or research tools comprise of the usage of the engineered cell that facilitate the scientific study of biological processes; or
(6) (a) providing an engineered or recombinant cell that enables monitoring for post cellular gene therapy and tracking for safety through the expression of exogenous molecules, or
(b) (the engineered or recombinant cell) comprising a nucleic acid encoding a protein that enables monitoring for post cellular gene therapy and tracking for safety through the expression of exogenous molecules, and optionally the nucleic acid is under the control of (operably linked to) a mechano-responsive promoter, optionally a YAP/TAZ mechanoresponsive promoter; or
(7) (a) providing an engineered or recombinant cell that directly or indirectly aids in treating or ameliorating a cancer, a cancer metastases, a tissue fibrosis, cell fate tracking, diabetes, wound healing, cosmetics, osteoporosis, regenerative medicine, or an immune disease,
(b) (the engineered or recombinant cell) comprising a nucleic acid encoding a protein that treats or ameliorates a cancer, a cancer metastases, a tissue fibrosis, cell fate tracking, diabetes, wound healing, cosmetics, osteoporosis, regenerative medicine, or an immune disease, and optionally the nucleic acid is under the control of (operably linked to) a mechano-responsive promoter, optionally a YAP/TAZ mechanoresponsive promoter,
wherein optionally the cancer or cancer metastases comprises a condition when cancer spreads into tissue other than its origination, and the tissue other than its origination has a sufficient mechanical modulus, or stiffness to activate (turn on) the mechano-responsive promoter,
and optionally the tissue fibrosis comprises a condition of excessive formation of fibrous connective tissue, and optionally the excessive formation of fibrous connective tissue has a sufficient mechanical modulus, or stiffness to activate (turn on) the mechano-responsive promoter,
and optionally the cell fate tracking comprises a method of detecting the fate of engineered cell in vivo,
and optionally the diabetes comprises prolonged high level of blood glucose, and optionally the wound healing comprises regeneration and remodeling of damaged tissue,
and optionally the cosmetics comprises improving appearance of the body, and optionally the osteoporosis comprises a decreased bone mass and density, and optionally the regenerative medicine comprises a process of replacing, engineering or regenerating human cells, tissues or organs to restore or establish normal function,
and optionally the immune disease comprises of a disease caused by a deficient or malfunctioned immune system.

2: An engineered or recombinant cell for use in treating, ameliorating, preventing or removing a scar tissue, wherein the cell comprises:
(a) an exogenous nucleic acid encoding a secreted enzyme capable of disrupting or removing a scar tissue,
wherein expression of the exogenous nucleic acid is under the control of (operably linked to) a mechano-responsive promoter (wherein the promoter is activated by an
increase in the stiffness of the cell's environment, or contact with a tissue or environment having an increased mechanical modulus, or stiffness), and optionally the mechano-responsive promoter comprises or is a YAP/TAZ mechanoresponsive promoter, or
(b) an endogenous nucleic acid encoding a secreted enzyme capable of disrupting or removing a scar tissue, wherein the endogenous nucleic acid is engineered to be operably linked to a mechano-responsive promoter, optionally by use of a CRISPR/Cas9 methodology or equivalent, or homologous recombination,
wherein optionally the engineered or recombinant cell is capable of targeting or binding to a fibrosis or a scar tissue, or is engineered to target or bind to a fibrosis or a scar tissue,
and optionally the engineered or recombinant cell is a stem cell, a fibroblast, an epithelial cell, or an immune cell, optionally a T cell, a lymphocyte or a megakaryocyte.

3: A method for treating, ameliorating, dissolving, preventing or removing a scar tissue or a fibrosis in an individual in need thereof, or
use of an engineered or recombinant cell for treating, ameliorating, preventing or removing a scar tissue a fibrosis, or
an engineered or recombinant cell for treating, ameliorating, preventing or removing a scar tissue a fibrosis, comprising:
(a) providing an engineered or recombinant cell of claim 2, and
(b) administering the cell to the individual in need thereof,
wherein optionally the fibrosis or scar treated, ameliorated, dissolved, prevented or removed comprises a fibrosis or scar associated with a fibrosis-related disease, optionally a lung, liver, kidney, heart or vessel fibrosis, or a wound-induced or surgical induced scar, or a scar induced by a myocardial infarction or a myocardial infection.

4: An engineered or recombinant cell for use in treating, ameliorating or preventing a condition responsive to an antibody or a chimeric antigen receptor (CAR), wherein the cell comprises:
(a) an exogenous nucleic acid encoding an antibody a chimeric antigen receptor (CAR), wherein the antibody or CAR can treat, ameliorate or prevent a condition responsive to an antibody or a chimeric antigen receptor (CAR),
wherein expression of the exogenous nucleic acid is under the control of (operably linked to) a mechano-responsive promoter (wherein the promoter is activated by an increase in the stiffness of the cell's environment, or contact with a tissue or environment having an increased mechanical modulus, or stiffness), and optionally the mechano-responsive promoter comprises or is a YAP/TAZ mechanoresponsive promoter, or
(b) an endogenous nucleic acid encoding an antibody, wherein the endogenous nucleic acid is engineered to be operably linked to a mechano-responsive promoter, optionally by use of a CRISPR/Cas9 methodology or equivalent, or homologous recombination,
wherein optionally the engineered or recombinant cell is capable of targeting or binding to a specific or a desired cell, organ or tissue, or is engineered to target or bind to a specific or a desired cell, organ or tissue, optionally the engineered or recombinant cell is capable of targeting or binding to a cancer or tumor, optionally a solid tumor, or a cancer metastasis,
and optionally the engineered or recombinant cell is a stem cell, a fibroblast, an epithelial cell, or an immune cell, optionally a T cell, a lymphocyte or a megakaryocyte.

5: A method for treating, ameliorating or preventing a condition responsive to an antibody or a chimeric antigen receptor (CAR) in an individual in need thereof, or use of an engineered or recombinant cell for treating, ameliorating or preventing a condition responsive to an antibody or a chimeric antigen receptor (CAR), or
an engineered or recombinant cell for treating, ameliorating or preventing a condition responsive to an antibody or a chimeric antigen receptor (CAR), comprising:
(a) providing an engineered or recombinant cell of claim 4, and
(b) administering the cell to the individual in need thereof,
wherein optionally the condition responsive to an antibody or a chimeric antigen receptor (CAR) is a cancer or tumor, optionally a solid tumor, or a cancer metastasis.

6: An engineered or recombinant cell for use in delivering a detectable probe or molecule, or a therapeutic molecule, to a targeted cell, organ or tissue in an individual in need thereof, wherein the cell comprises:
(a) an exogenous nucleic acid encoding a detectable probe or molecule, or a therapeutic molecule,
wherein expression of the exogenous nucleic acid is under the control of (operably linked to) a mechano-responsive promoter (wherein the promoter is activated by an increase in the stiffness of the cell's environment, or contact with a tissue or environment having an increased mechanical modulus, or stiffness), and optionally the mechano-responsive promoter comprises or is a YAP/TAZ mechanoresponsive promoter, or
(b) an endogenous nucleic acid encoding a therapeutic molecule or a detectable molecule, wherein the endogenous nucleic acid is engineered to be operably linked to a mechano-responsive promoter, optionally by use of a CRISPR/Cas9 methodology or equivalent, or homologous recombination,
wherein optionally the engineered or recombinant cell is capable of targeting or binding to a specific or a desired cell, organ or tissue, or is engineered to target or bind to a specific or a desired cell, organ or tissue, optionally the engineered or recombinant cell is capable of targeting or binding to a cancer or tumor, optionally a solid tumor, or a cancer metastasis,
wherein optionally the detectable probe or molecule comprises a fluorescent protein, optionally an enhanced green fluorescent protein (eGFP), a beta-galactosidase (beta-gal) (optionally an E. coli beta-gal), a horseradish peroxidase (HRP) or a luciferase, and optionally the therapeutic molecule comprises a cytosine deaminase (CD),
and optionally the detectable probe or molecule is a secreted detectable probe or molecule, and optionally after secretion by the cell the detectable probe or molecule is detectable in a body fluid, optionally blood or urine,
and optionally the engineered or recombinant cell is a stem cell, a fibroblast, an epithelial cell, or an immune cell, optionally a T cell, a lymphocyte or a megakaryocyte.

7: A method for delivering a detectable probe or a therapeutic molecule to a targeted cell, organ or tissue in an individual in need thereof, or
use of a detectable probe or a therapeutic molecule for detecting or treating a targeted cell, organ or tissue in an individual in need thereof, or
an engineered or recombinant cell for detecting or treating a targeted cell, organ or tissue in an individual in need thereof, comprising:
(a) providing an engineered or recombinant cell of claim 6, and
(b) administering the cell to the individual in need thereof,
wherein optionally a cancer or tumor, optionally a solid tumor, or a cancer metastasis, is treated or detected by the detectable probe or the therapeutic molecule.

8: A non-human transgenic animal comprising an engineered or recombinant cell of claim 1.

9: The method of claim 1, wherein the engineered or recombinant cell is an immune cell, optionally a T cell, a mesenchymal stem cell (MSC), a neural stem cell (NSC), a hematopoietic stem cell (HSC) or a microorganism cell, optionally a bacterial cell.

10: The method of claim 1, wherein the engineered or recombinant cell is engineered to comprise at least one exogenous nucleic acid having the ability to express: a therapeutic agent, a converter enzyme, a pro-enzyme, an antibody, an exogenous protein, an exogenous nanoparticle, a homing agent, or any molecule that originally does not exist in the cell, and expression of the nucleic acid is under the control of or is operably linked to a mechano-responsive promoter, or wherein the promoter is activated by an increase in the stiffness of the cell's environment, or contact with a tissue or environment having an increased mechanical modulus, or stiffness, and optionally the mechano-responsive promoter comprises or is a YAP/TAZ mechanoresponsive promoter.

11: The method of claim 1, wherein the engineered or recombinant cell comprises a homing agent, or is engineered to comprise an exogenous homing agent, comprising a protein or any form of molecule that facilitates or enhances the migration of the engineered or recombinant cell to certain or desired niche, including but not limited to a tumor niche, and optionally the homing agent is encoded by a nucleic acid under the control of or is operably linked to a mechano-responsive promoter, optionally a YAP/TAZ mechanoresponsive promoter, and optionally the engineered or recombinant cell comprises a therapeutic agent, or is engineered to comprise an exogenous therapeutic agent, optionally a direct therapeutic agent, comprising a protein enzyme or any form of molecule that has a direct toxic or beneficial effect to other cells, and optionally the therapeutic agent is encoded by a nucleic acid under the control of or is operably linked to a mechano-responsive promoter, optionally a YAP/TAZ mechanoresponsive promoter.

12: The method of claim 1, wherein the engineered or recombinant cell comprises a converter enzyme, or is engineered to comprise an exogenous converter enzyme, comprising a protein enzyme or any form of molecule that is capable of converting a toxic, inactive, or ineffective molecule into a diagnostic or therapeutic agent, and optionally the converter enzyme is encoded by a nucleic acid under the control of or is operably linked to a mechanoresponsive promoter, optionally a YAP/TAZ mechanoresponsive promoter.

13: The method of claim 1, wherein the engineered or recombinant cell comprises a pro-enzyme, or is engineered to comprise an exogenous pro-enzyme, comprising a protein enzyme or any form of molecule that is capable of being converted into a direct therapeutic agent, and optionally the pro-enzyme is encoded by a nucleic acid under the control of or is operably linked to a mechano-responsive promoter, optionally a YAP/TAZ mechanoresponsive promoter.

14: The method of claim 1, wherein the engineered or recombinant cell comprises an antibody or antigen binding agent, or is engineered to comprise an exogenous antibody or antigen binding agent, wherein the antibody or antigen binding agent comprises a protein antibody or any form of molecule that is capable of binding to specific target, and optionally the antibody or antigen binding agent is encoded by a nucleic acid under the control of or is operably linked to a mechano-responsive promoter, optionally a YAP/TAZ mechanoresponsive promoter.

15: The method of claim 1, wherein the engineered or recombinant cell comprises an exogenous protein that is originated from a species other than the engineered cell, or is modified from the natural form of the protein, and the exogenous protein is encoded by a nucleic acid under the control of or is operably linked to a mechano-responsive promoter, optionally a YAP/TAZ mechanoresponsive promoter.

16: The method of claim 1, wherein the engineered or recombinant cell comprises an exogenous device, optionally a nanoparticle or comprising any molecule that the original cell does not possess,
and optionally the mechano-responsive promoter, optionally a YAP/TAZ mechanoresponsive promoter is engineered into the cell to drive an endogenous nucleic acid of interest, and optionally the mechanoresponsive promoter is engineered into the cell using CRISPR/Cas9 or equivalent methodology.

17: A multiplexed system or a device comprising, incorporating or using an engineered cell or recombinant cell of claim 1.

18: A method for targeting, to detecting or monitoring, or for treating abnormal cells or tissue of diseases, comprising use of an engineered cell or recombinant cell of claim 1.
